SEQUENCE LISTING (31 sequences)

SEQ ID NO: 1 - DNA, 22 bp, Nicotiana benthiama
gatgggattgcacgtgtttatg

SEQ ID NO: 2 - DNA, 22 bp, Nicotiana benthiama
taatggaatgcacgcattaata

SEQ ID NO: 3 - protein, 354 aa, Artificial Sequence
  Synthesized
MNTKYNKEFLLYLAGFVDGDGSITACIQPGQHCKFKHVLRLRFGVSQKTQRRWFLDKLVDEIGVGYVYDLGSVSEYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSIFAAILPQQPRKFKHALQLFFVVTQKTQRRWFLDKLVDEIGVGYVVDRGSVSDYKLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 4 - DNA, 1062 bp, Artificial Sequence
  Synthesized
atgaatacaaaatataataaagagttcttactctacttagcagggtttgtagacggtgacggttccatcactgcctgtatccagcctgggcaacattgtaagttcaagcacgtgctgaggctccggttcggggtcagtcagaagacacagcgccgttggttcctcgacaagctggtggacgagatcggtgtgggttacgtgtatgaccttggcagcgtctccgagtaccggctgtcccagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgccgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacagcgacggctccatctttgccgcgatccttcctcagcaacctaggaagttcaagcacgctctgcagctctttttcgttgtcacgcagaagacacagcgccgttggttcctcgacaagctggtggacgagatcggtgtgggttacgtggtggaccggggcagcgtctccgattacaagctgtcccagatcaaacctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccc

SEQ ID NO: 5 - protein, 354 aa, Artificial Sequence
  Synthesized
MNTKYNKEFLLYLAGFVDGDGSICAVIRPSQAYKFKHQLQLVFAVAQKTQRRWFLDKLVDEIGVGYVTDNGSVSLYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSIFAAIQPCQRAKFKHLLHLRFDVAQKTQRRWFLDKLVDEIGVGYVADNGSVSNYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 6 - DNA, 1062 bp, Artificial Sequence
  Synthesized
atgaatacaaaatataataaagagttcttactctacttagcagggtttgtagacggtgacggttccatctgtgcagtgatccggccttctcaagcgtataagttcaagcaccagctgcagctcgtttttgctgtcgcgcaaaagacacagcgccgttggttcctcgacaagctggtggacgagatcggtgtgggttacgtgactgacaatggcagcgtctccctgtaccggctgtcccagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttgccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgactccgacggctccatctttgccgcgatccagccttgtcaacgggcgaagttcaagcaccttctgcatctccgtttcgatgtcgctcagaagacacagcgccgttggttcctcgacaagctggtggacgagatcggtgtgggttacgtggcggacaatggcagcgtctccaattacaggctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccc

SEQ ID NO: 7 - protein, 66 aa, Nicotiana benthiama
MASRRLLTSLLRQSAQRGGGPISRSLGNSIPKSAARASSRASPKGFLLNRAVQYATSAAAPASQPS

SEQ ID NO: 8 - protein, 54 aa, Nicotiana benthiama
MASRRLLTSLLRQSAQRGGGPISRSLGNSIPKSAARASSRASPKGFLLNRAVQY

SEQ ID NO: 9 - protein, 29 aa, Saccharomyces cerevisiae
MLSLRQSIRFFKPATRTLCSSRYLLQQKP

SEQ ID NO: 10 - protein, 42 aa, Arabidopsis thaliana
MGSSFSASFTNSTTAAAVPPPSPPSSPSRSNVKSNGEERPRF

SEQ ID NO: 11 - protein, 98 aa, Artificial Sequence
  Synthesized
MSVLTPLLLRGLTGSARRLPVPRAKIHSLPPEGKLMASTRVLASRLASQMAASAKVARPAVRVAQVSKRTIQTGSPLQTLKRTQMTSIVNATTRQAFQ

SEQ ID NO: 12 - protein, 18 aa, Mouse parovirus NS2
VDEMTKKFGTLTIHDTEK

SEQ ID NO: 13 - protein, 10 aa, Artificial Sequence
  Synthesized
LGAGLGALGL

SEQ ID NO: 14 - DNA, 23 bp, Artificial Sequence
  Synthesized
gatgggattgcacgcattaataa

SEQ ID NO: 15 - DNA, 856 bp, Artificial Sequence
  Synthesized
atggaactttctccccgagctgcggaactaacaagtctattagaaagtcgaattagcaacttttacaccaattttcaagtggatgagatcggtcgagtggtctcagttggagatgggattgcacgcattaataatctatgatgatcttagtaaacaggcggtagcatatcgacaaatgtcattattgttacgccgaccaccaggtcgtgaggctttcccaggggatgttttctatttacattcccgtctcttagaaagagcggctaaacgatcggaccagacaggcgcaggtagcttgaccgccttacccgtcattgaaacacaggctggagacgtatcggcctatattcccaccaatgtgatccccattactgatggacaaatctgtttggaaacagagctcttttatcgcggaattagacctgcgattaacgtcggcttatctgtcagtcgcgtcgggtctgccgctcagttgaaaactatgaaacaagtctgcggtagttcaaaactggaattggcacaatatcgcgaagtggccgcccttgctcaatttggctcagaccttgatgctgcgactcaggcattactcaatagaggtgcaaggctgacagaagtaccgaaacaaccacaatatgcaccactgccaattgaaaaacaaattctagtcatttatgcagctgtcaatggattctgtgatcgaatgccactagacagaatttctcaatatgagagagccattccaaatagtgtcaaaccagaattactacaatcctttttagaaaaaggtggcttaactaacgaaagaaagatggaaccagatacattcttaaaagaaagtgctttagcttttatttaa

SEQ ID NO: 16 - DNA, 22 bp, Nicotiana benthiama
ctaccctaacgtgcacaaatac

SEQ ID NO: 17 - DNA, 22 bp, Nicotiana benthiama
attaccttacgtgcgtaattat

SEQ ID NO: 18 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 19 - DNA, 1530 bp, Artificial Sequence
  Synthesized
atggaactttctccccgagctgcggaactaacaagtctattagaaagtcgaattagcaacttttacaccaattttcaagtggatgagatcggtcgagtggtctcagttggagatgggattgcacgtgtttatggattgaacgagattcaagctggggaaatggttgaatttgccagcggtgtgaaaggaatagccttgaatcttgagaatgagaatgtagggattgttgtctttggtagtgatactgctattaaagaaggagatcttgtcaagcgcactggatctattgtggatgttcctgcgggaaaggctatgctagggcgtgtggtcgatggcttgggagtacctattgatggaaggggggctctaagcgatcacgagcgaagacgtgtcgaagtgaaagcccctggtattattgaacgtaaatctgtgcacgagcctatgcaaacagggttaaaagcggtagatagcctggttcctataggtcgtggtcaacgagaacttataatcggggaccgacaaactggaaaaactgctattgctatcgataccatattaaaccaaaagcaactgaactcaagggccacctctgagagtgagacattgtattgtgtctatgtagcgattggacagaaacgctcaactgtggcacaattagttcaaattctttcagaagcgaatgctttggaatattctattcttgtagcagccaccgcttcggatcctgctcctctacaatttttggccccatattctgggtgtgccatgggggaatatttccgcgataatggaatgcacgcattaataatctatgatgatcttagtaaacaggcggtagcatatcgacaaatgtcattattgttacgccgaccaccaggtcgtgaggctttcccaggggatgttttctatttacattcccgtctcttagaaagagcggctaaacgatcggaccagacaggcgcaggtagcttgaccgccttacccgtcattgaaacacaggctggagacgtatcggcctatattcccaccaatgtgatctccattactgatggacaaatctgtttggaaacagagctcttttatcgcggaattagacctgcgattaacgtcggcttatctgtcagtcgcgtcgggtctgccgctcagttgaaaactatgaaacaagtctgcggtagtttaaaactggaattggcacaatatcgcgaagtggccgcctttgctcaatttggctcagaccttgatgctgcgactcaggcattactcaatagaggtgcaaggctgacagaagtactgaaacaaccacaatatgcaccactgccaattgaaaaacaaattctagtcatttatgcagctgtcaatggattctgtgatcgaatgccactagacagaatttctcaatatgagagagccattctaaatagtgtcaaaccagaattactacaatcctttttagaaaaaggtggcttaactaacgaaagaaagatggaactagatacattcttaaaagaaagtgctttagcttttatttaa

SEQ ID NO: 20 - DNA, 2638 bp, Artificial Sequence
  Synthesized
ggatccactatggcttctcggaggcttctcacctctctcctccgtcaatcggctcaacgtggcggcggtccaatttcccgatccttgggaaactccatccctaaatccgctgcacgcgcctcttcacgcgcgtcccctaagggattcctcttaaaccgcgccgtacagtacgctacctctgcagcagcacccgcatctcagccatccatggaactctcaccaagggcagcagaacttacttcactcctcgaatctagaatctcaaacttctacacaaactttcaggttgatgaaattggaagagttgtttcagttggagatggtattgctagggtttatggacttaatgagattcaagctggtgaaatggttgagtttgcttctggagttaagggtatcgctttgaatcttgaaaatgagaacgttggaatcgttgtttttggttcagatactgctattaaggagggagatttggttaaaagaacaggttctattgttgatgttccagctggaaaagctatgcttggtagagttgttgatggattgggtgttcctattgatggaaggggtgctttgtctgatcatgaaagaaggagagttgaggttaaggctccaggaattattgaaagaaaatcagttcatgagcctatgcaaactggtcttaaggctgttgattctttggttccaattggaaggggtcaaagagaacttattattggagataggcaaactggaaagactgctatcgctatcgatacaatccttaaccaaaagcaattgaattcaagagctacttctgaatcagagacattgtattgtgtttacgttgctattggacaaaaaaggtctactgttgctcaacttgttcaaattttgtctgaagctaatgctcttgagtattcaattttggttgctgctacagcttctgatccagctcctcttcaatttttggctccatattcaggatgcgctatgggtgaatactttagagataatggtatgcatgctcttattatatatgatgatttgtcaaagcaagctgttgcttacagacaaatgtctcttttgcttagaagaccaccaggaagggaagcttttcctggagatgttttctatcttcattctaggttgcttgagagagctgctaaaaggtctgatcaaactggagctggttcacttacagctttgccagttattgagactcaagctggagatgtttcagcttacatccctactaacgttatctctatcacagatggacaaatttgtcttgaaacagagttgttttacaggggtattagaccagctattaatgttggactttctgtttcaagagttggttcagctgctcaattgaagactatgaaacaagtttgcggttctttgaagcttgaattggctcaatatagggaggttgctgcttttgctcaatttggatcagatttggatgctgctactcaagctttgcttaataggggtgctagacttacagaagttttgaagcaacctcaatatgctccacttcctatcgagaagcaaattcttgttatatatgctgctgttaatggtttctgtgatagaatgccacttgataggatctctcaatacgaaagagctatcttgaactcagttaagcctgaattgcttcaatcttttcttgagaaaggaggtttgacaaacgagaggaagatggagctcgatacttttctcaaggagtccgcactcgcattcatctgaagatctatagattataaacttctgtgactttcttttcttctctttgccaaaataatttagtttgtgacatcccggatttttttggaggaccaagaggtccagaattctggttttgttttacatccaatgcgagattatagagacatgcagccaagcctttgttgccagagacccccttttctgttatgtcacataataaagggggtaaatggtgatcttgtatatctgattttcaagtctttttcgagaattttggattccctgattatcaaatgcctttctgaaacgctttctttctatatgtggttaacttcacgtccattttatccattctgccttgaaagcttcaatgtaataggagcagttaatatgctaagcggatacaaatcatatttcttggcgcgacttaattttattgcagtatgaatacttgtagaaaatatgagtatttgactaagcttataagcggcaaaccagcttataagtcacttttactttacttcatctacgcgtttggtaaaattaaaagtgttaagtctagtgcttgtaagcttttaagtcttaagttgtcataagttggtcacatctaaatttgaactgcccctgcccccatcttccccaaaagaatctgcattcacgctaagaggttatgcaaactctttagggctaaagaattgtcgtcaaattataactaaagccaaagttctaaaatatgtcttacagagataatgtctagagaataataagatttatatgcagagacaattcctttagattcctttaggatgataataacacacgccagacatacattatagtggaaaaaataatgaacaggaaattacacccaataaccttgaacatatatggtcttgattttttgattctttgtcccggtacc

SEQ ID NO: 21 - DNA, 3491 bp, Artificial Sequence
  Synthesized
gtcgacgagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgaaccaggatccactatggcttctcggaggcttctcacctctctcctccgtcaatcggctcaacgtggcggcggtccaatttcccgatccttgggaaactccatccctaaatccgctgcacgcgcctcttcacgcgcgtcccctaagggattcctcttaaaccgcgccgtacagtacgctacctctgcagcagcacccgcatctcagccatccatggaactctcaccaagggcagcagaacttacttcactcctcgaatctagaatctcaaacttctacacaaactttcaggttgatgaaattggaagagttgtttcagttggagatggtattgctagggtttatggacttaatgagattcaagctggtgaaatggttgagtttgcttctggagttaagggtatcgctttgaatcttgaaaatgagaacgttggaatcgttgtttttggttcagatactgctattaaggagggagatttggttaaaagaacaggttctattgttgatgttccagctggaaaagctatgcttggtagagttgttgatggattgggtgttcctattgatggaaggggtgctttgtctgatcatgaaagaaggagagttgaggttaaggctccaggaattattgaaagaaaatcagttcatgagcctatgcaaactggtcttaaggctgttgattctttggttccaattggaaggggtcaaagagaacttattattggagataggcaaactggaaagactgctatcgctatcgatacaatccttaaccaaaagcaattgaattcaagagctacttctgaatcagagacattgtattgtgtttacgttgctattggacaaaaaaggtctactgttgctcaacttgttcaaattttgtctgaagctaatgctcttgagtattcaattttggttgctgctacagcttctgatccagctcctcttcaatttttggctccatattcaggatgcgctatgggtgaatactttagagataatggtatgcatgctcttattatatatgatgatttgtcaaagcaagctgttgcttacagacaaatgtctcttttgcttagaagaccaccaggaagggaagcttttcctggagatgttttctatcttcattctaggttgcttgagagagctgctaaaaggtctgatcaaactggagctggttcacttacagctttgccagttattgagactcaagctggagatgtttcagcttacatccctactaacgttatctctatcacagatggacaaatttgtcttgaaacagagttgttttacaggggtattagaccagctattaatgttggactttctgtttcaagagttggttcagctgctcaattgaagactatgaaacaagtttgcggttctttgaagcttgaattggctcaatatagggaggttgctgcttttgctcaatttggatcagatttggatgctgctactcaagctttgcttaataggggtgctagacttacagaagttttgaagcaacctcaatatgctccacttcctatcgagaagcaaattcttgttatatatgctgctgttaatggtttctgtgatagaatgccacttgataggatctctcaatacgaaagagctatcttgaactcagttaagcctgaattgcttcaatcttttcttgagaaaggaggtttgacaaacgagaggaagatggagctcgatacttttctcaaggagtccgcactcgcattcatctgaagatctatagattataaacttctgtgactttcttttcttctctttgccaaaataatttagtttgtgacatcccggatttttttggaggaccaagaggtccagaattctggttttgttttacatccaatgcgagattatagagacatgcagccaagcctttgttgccagagacccccttttctgttatgtcacataataaagggggtaaatggtgatcttgtatatctgattttcaagtctttttcgagaattttggattccctgattatcaaatgcctttctgaaacgctttctttctatatgtggttaacttcacgtccattttatccattctgccttgaaagcttcaatgtaataggagcagttaatatgctaagcggatacaaatcatatttcttggcgcgacttaattttattgcagtatgaatacttgtagaaaatatgagtatttgactaagcttataagcggcaaaccagcttataagtcacttttactttacttcatctacgcgtttggtaaaattaaaagtgttaagtctagtgcttgtaagcttttaagtcttaagttgtcataagttggtcacatctaaatttgaactgcccctgcccccatcttccccaaaagaatctgcattcacgctaagaggttatgcaaactctttagggctaaagaattgtcgtcaaattataactaaagccaaagttctaaaatatgtcttacagagataatgtctagagaataataagatttatatgcagagacaattcctttagattcctttaggatgataataacacacgccagacatacattatagtggaaaaaataatgaacaggaaattacacccaataaccttgaacatatatggtcttgattttttgattctttgtcccggtacc

SEQ ID NO: 22 - DNA, 4841 bp, Artificial Sequence
  Synthesized
gtcgacatagacccccaaagggattgttcaaactaaacatagatgcaagtttcaacaaaaatttgcaaaattgtgccctgggtggagtaatcagaaattaatgctaatggtcactggatagttggctttgcaaaatcagctcatgcacgtgggtcactacatgctgagatcaaaacattactagcaggacttaaaacaactcatacttgggggatgttccctctccaaattgaacaatattaattattcttcttaatttgtaccaagttatattacgtatccttaaaatcgcatataaattcaactgttatccgattttagggtaaacagtttggcgatcaccgtggggctaaggataatggtgattacctggtacaaactttcatgacacacactattttacacttgttctttgaagtgtctttgattacaggactaaaaaatatcaaactctcagtcagcacctctacacattgacaatgatttcggccgccacgacgaaaatgataacatagcagcagggaacgatgtaccagtattagcctcgatgaattttcggccgcggatctagtggacgtcagttcacatataaccatcaacgggaatttagccgtcgatcccgaaggcaccatctgtggggatgctcgatcagccgctcaaagcgcatctggcggtgaagatggtaggataagcctgtaggtgatcttcgaaatgttgcaggctcagcaggcagcaatagcccagctccaaaaccagaatcacacaccaagcagggttgagctcgagccatcccaggaagttgtacacatggtcgaatcggtttcaagaaaatcgagcaagaaagagtcgaagaccaacccatcaatcatgaagatgctcgaagaactaacgaagcgaattgagtcggggaaaaaaaagatcgaggcaaacgacaagaaggtggaaacttgtaactccagggtcgatcaaatcccgggggcaccaccaatattgaaaggactagattccaaaaagttcatacaaaaaccttcccccctcccccgagcacggctgcgaagccaatccccaagaagttccgcatgcccgagattcctaagtataatggaatgactgatccgaatgagcacgtcacctcttacacgtgtgctatcaaagggaatgatctagaggatgacgagatcgaatcctgtcaatccaatagggtaataatctttatgctaacattgtcaacaaatgcaggttattaatgcaccaacaaaatgaggtaatcatccagcacgagttcaggcaagggaataaggtagctcaccaaatggcaaagaaggcaactgctgatctcaggaaggagaaagtttttgtggaaccaccagatattgtaaaaaatctagtagaaagtgatttaccagaacaatatatttttggaaaactactatcttatgatacttgtaaaaatttagcaagcctaggtaaccaaagtgtcctatgcgacactaactatgaatctcatgtactgcatgttccttaacctttaatatatatatttcctgtttgctcaaaaaaaaaaatattagttacttgtagttagtcttaaaacaattaaatagtgtaatttttgtttataatatcggtttataaaaaataaaccttaatgaaatttgcgcatatttgagtcaagtttatgacctttttgcgtgatatacaaccttacattaccatccaactatgttatatgagttttttatttattagagattgtacgtatcaagtgcaaagaatttttttactcgtatttgttatttgcatcaaattaataaatacaaaatactatgttttgattcttgcacaaattttttattttaattatgattaaactatatgttctcgtactattttaatttcatagattaatctataacagtataactattgagtctcactttgtgcaagtgtggagcacatatttgaaaacataaggatgcccaagttattcgccaaaaaagttggaataaaagcgaaaaaggaaaacaaagaaaaaaaagaacaaattcaaatgctcctcggttttttaagcacagtaaagcagcaatcggatcacacagtcgcacagtgggctcttgataaatcagctcttcatatttcccacaaaccctagcagtctctttctctctctactcctttcatcctctctctaaccaaaccctccggatccactatggcttctcggaggcttctcacctctctcctccgtcaatcggctcaacgtggcggcggtccaatttcccgatccttgggaaactccatccctaaatccgctgcacgcgcctcttcacgcgcgtcccctaagggattcctcttaaaccgcgccgtacagtacgctacctctgcagcagcacccgcatctcagccatccatggaactctcaccaagggcagcagaacttacttcactcctcgaatctagaatctcaaacttctacacaaactttcaggttgatgaaattggaagagttgtttcagttggagatggtattgctagggtttatggacttaatgagattcaagctggtgaaatggttgagtttgcttctggagttaagggtatcgctttgaatcttgaaaatgagaacgttggaatcgttgtttttggttcagatactgctattaaggagggagatttggttaaaagaacaggttctattgttgatgttccagctggaaaagctatgcttggtagagttgttgatggattgggtgttcctattgatggaaggggtgctttgtctgatcatgaaagaaggagagttgaggttaaggctccaggaattattgaaagaaaatcagttcatgagcctatgcaaactggtcttaaggctgttgattctttggttccaattggaaggggtcaaagagaacttattattggagataggcaaactggaaagactgctatcgctatcgatacaatccttaaccaaaagcaattgaattcaagagctacttctgaatcagagacattgtattgtgtttacgttgctattggacaaaaaaggtctactgttgctcaacttgttcaaattttgtctgaagctaatgctcttgagtattcaattttggttgctgctacagcttctgatccagctcctcttcaatttttggctccatattcaggatgcgctatgggtgaatactttagagataatggtatgcatgctcttattatatatgatgatttgtcaaagcaagctgttgcttacagacaaatgtctcttttgcttagaagaccaccaggaagggaagcttttcctggagatgttttctatcttcattctaggttgcttgagagagctgctaaaaggtctgatcaaactggagctggttcacttacagctttgccagttattgagactcaagctggagatgtttcagcttacatccctactaacgttatctctatcacagatggacaaatttgtcttgaaacagagttgttttacaggggtattagaccagctattaatgttggactttctgtttcaagagttggttcagctgctcaattgaagactatgaaacaagtttgcggttctttgaagcttgaattggctcaatatagggaggttgctgcttttgctcaatttggatcagatttggatgctgctactcaagctttgcttaataggggtgctagacttacagaagttttgaagcaacctcaatatgctccacttcctatcgagaagcaaattcttgttatatatgctgctgttaatggtttctgtgatagaatgccacttgataggatctctcaatacgaaagagctatcttgaactcagttaagcctgaattgcttcaatcttttcttgagaaaggaggtttgacaaacgagaggaagatggagctcgatacttttctcaaggagtccgcactcgcattcatctgaagatctatagattataaacttctgtgactttcttttcttctctttgccaaaataatttagtttgtgacatcccggatttttttggaggaccaagaggtccagaattctggttttgttttacatccaatgcgagattatagagacatgcagccaagcctttgttgccagagacccccttttctgttatgtcacataataaagggggtaaatggtgatcttgtatatctgattttcaagtctttttcgagaattttggattccctgattatcaaatgcctttctgaaacgctttctttctatatgtggttaacttcacgtccattttatccattctgccttgaaagcttcaatgtaataggagcagttaatatgctaagcggatacaaatcatatttcttggcgcgacttaattttattgcagtatgaatacttgtagaaaatatgagtatttgactaagcttataagcggcaaaccagcttataagtcacttttactttacttcatctacgcgtttggtaaaattaaaagtgttaagtctagtgcttgtaagcttttaagtcttaagttgtcataagttggtcacatctaaatttgaactgcccctgcccccatcttccccaaaagaatctgcattcacgctaagaggttatgcaaactctttagggctaaagaattgtcgtcaaattataactaaagccaaagttctaaaatatgtcttacagagataatgtctagagaataataagatttatatgcagagacaattcctttagattcctttaggatgataataacacacgccagacatacattatagtggaaaaaataatgaacaggaaattacacccaataaccttgaacatatatggtcttgattttttgattctttgtcccggtacc

SEQ ID NO: 23 - DNA, 23 bp, Artificial Sequence
  Synthesized
ggatccactatggcttctcggag

SEQ ID NO: 24 - DNA, 23 bp, Artificial Sequence
  Synthesized
ctatagatcttcagatgaatgcg

SEQ ID NO: 25 - DNA, 19 bp, Artificial Sequence
  Synthesized
agcaccctgttcttctcac

SEQ ID NO: 26 - DNA, 19 bp, Artificial Sequence
  Synthesized
gtcaagctcctgctcgtag

SEQ ID NO: 27 - DNA, 20 bp, Artificial Sequence
  Synthesized
caagtggatgagatcggtcg

SEQ ID NO: 28 - DNA, 20 bp, Artificial Sequence
  Synthesized
actgacagataagccgacgt

SEQ ID NO: 29 - DNA, 20 bp, Artificial Sequence
  Synthesized
atccgggccttaatccttgc

SEQ ID NO: 30 - DNA, 20 bp, Artificial Sequence
  Synthesized
tgcgaggggaaaacttttgt

SEQ ID NO: 31 - protein, 354 aa, Artificial Sequence
  Synthesized
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

(19) United States
(12) Patent Application Publication
(10) Pub. No.: US 2024/0141311 A1
(43) Pub. Date: May 2, 2024

(54) COMPOSITIONS AND METHODS FOR GENERATING MALE STERILE PLANTS

(71) Applicants: Precision BioSciences, Inc., Durham, NC (US); North Carolina State University, Raleigh, NC (US)

(72) Inventors: Ralph Dewey, Apex, NC (US); Derek Jantz, Durham, NC (US); James Jefferson Smith, Morrisville, NC (US); Ginger H. Tomberlin, Cary, NC (US); John Morris, Raleigh, NC (US); Anna Matthiadis, Durham, NC (US); Wendy Shoop, Durham, NC (US)

(73) Assignee: North Carolina State University, Raleigh, NC (US)

(21) Appl. No.: 18/556,814

(22) PCT Filed: Apr. 22, 2022
(86) PCT No.: PCT/US2022/025965

§ 371 Date: Oct. 23, 2023

Related U.S. Application Data
(60) Provisional application No. 63/178,281, filed on Apr. 22, 2021.
(60) Provisional application No. 63/178,285, filed on Apr. 22, 2021.
(60) Provisional application No. 63/178,290, filed on Apr. 22, 2021.

(51) Int. Cl. C12N 9/22; C12N 15/82

(52) U.S. Cl. CPC C12N 9/22; C12N 15/8209; C12N 15/8289; C07K 2319/07

(57) ABSTRACT

Disclosed herein are recombinant meganucleases engineered to bind and cleave a recognition sequence in the mitochondrial DNA (mtDNA) of a eukaryotic cell, such as a plant cell. The disclosure further relates to the use of such recombinant meganucleases in methods for producing genetically-modified eukaryotic cells, and to a population of genetically-modified eukaryotic cells wherein the mtDNA has been having modified or edited.

### FIELD OF THE INVENTION

The present disclosure relates to the field of molecular biology and recombinant nucleic acid technology. In particular, the present disclosure relates to recombinant meganucleases engineered to recognize and cleave recognition sequences found in the plant mitochondrial genome. The present disclosure further relates to the use of such recombinant meganucleases in methods for producing genetically-modified eukaryotic cells, and to a population of genetically-modified eukaryotic cells wherein the mitochondrial DNA has been modified.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 22, 2022 is named P89339_0138_7_SeqList_4-22-22.txt, and is 37.1 kb in size.

### BACKGROUND OF THE INVENTION

Compared to the small, compact structure of human and animal mitochondrial genomes (typically ˜16.5 kb in size), plant mitochondrial genomes are much larger, ranging from 200 kb to greater than 10 Mb. Plant mitochondrial genomes are commonly depicted as large “master circles” representing the complete genome. In reality, however, the genetic information is distributed across populations of subgenomic circles, or even linear subgenomic fragments, delineated by long repeat regions that are recombinationally active, enabling the formation and maintenance of the subgenomes in differing stoichiometries. Mitochondria of angiosperms are predicted to contain 50-60 genes, including those encoding tRNAs, rRNAs, ribosomal proteins, certain subunits of the electron transport chain, the ATP synthase complex, and ORFs of unknown function. Investigating of the function and regulation of specific mitochondrial genes has been difficult due to the lack of mitochondrial transformation methodologies for multicellular eukaryotic species. In the absence of such a transformation system, the development of a facile means to edit the mitochondrial genomes of higher eukaryotes could fill this gap, and enable functional analyses of the genes found within this organelle.

Of all the various traits associated with the mitochondrial genomes of higher plants, the one that has by far received the most attention is cytoplasmic male sterility (CMS). Collectively, hybrid seed markets represent a multi-billion dollar worldwide industry. The CMS trait has been pivotal to this industry, being successfully exploited toward facilitating hybrid seed production in numerous self-pollinating crop species, including maize, canola, sorghum, rice, cotton, as well as numerous vegetable crops. The key feature that has made CMS-based systems of hybrid seed production (when coupled with an appropriate restorer gene) superior to strategies based on nuclear male-sterile genes, or any of a number of transgene-based strategies that have been devised over the years, is the strict maternal inheritance of the CMS trait. This feature has enabled breeders within the seed industry to utilize simple planting and crossing schemes to produce virtually pure lots of hybrid seed with negligible amounts of contaminating self-pollinated seed.

Despite its current value toward hybrid seed production, there remains great opportunity in further exploiting the CMS trait since it is currently being deployed at large scale only in select crop species. Among the reasons CMS systems are not more widely deployed include the following: (1) some crop species lack a well characterized CMS trait and/or restorer-of-fertility (Rf) gene; (2) in some species where such a system has been identified, it is not commercially deployed because the CMS trait is too leaky, or the fertility restoration under field conditions is not robust enough; or (3) in many species the CMS trait is associated with undesirable agronomic traits such as reduced yield, disease susceptibility, low seed set, or low germination. For example, although a CMS system was used almost universally to produce hybrid maize seed (the largest of all hybrid seed markets) in the U.S. throughout the 1950s and 60s, it was discontinued after it became apparent that this specific CMS trait (designated CMS-T) caused the crop to be very susceptible to the disease Southern Corn Leaf Blight. The inability to use the CMS trait to produce hybrid corn seed has forced the industry to use hand and mechanical detasseling instead, adding over $250 million a year to the costs of seed production in the U.S. alone (see 2010 APHIS filing by DuPont-Pioneer entitled “Pioneer Hi-Bred International, Inc. Seed Production Technology (SPT) Process DP-32138-1 Corn”). As another example, in rice the use of CMS systems has been pivotal in the commercial deployment of hybrid rice varieties. These hybrids have been instrumental in enabling greater total grain production on fewer total hectares of cultivated land from 1975 to the present. Despite this success, the full potential of hybrid rice production has not yet been met, as the majority of the available rice germplasm cannot be used for hybrid seed production due to negative agronomic associations between those lines and the existing CMS traits.

As mentioned above, the maternal inheritance of the CMS trait is a critical factor in the successful deployment of the trait in hybrid seed production. In all plant species investigated to date, the mutations responsible for conferring CMS have been shown to reside in the mitochondrial genome, an organelle that is maternally inherited in angiosperms.

New CMS traits could be developed if one could inactivate the function of an essential mitochondrial gene in a manner such that its function could be compensated for by a nuclear gene in all stages of plant development except anthesis. To date, however, all reports of successful genome editing of the mitochondrial genome in a higher plant have involved nonessential or redundant mitochondrial genes. This is presumably because knocking out the function of an essential gene would have been lethal. There is thus a need for the targeted editing of an essential mtDNA in a way in which the plant could still survive as this would open up the possibility for creating stable, maternally inherited male sterile plant varieties.

### SUMMARY OF THE INVENTION

Provided herein are compositions and methods for editing of mitochondrial genome. To date, all other attempts at mitochondrial genome editing have targeted nonessential or redundant genes, and thus would not be expected to be capable of generating CMS phenotypes. The present invention demonstrates for the first time that homing endonucleases allow the targeted knockout of an essential gene in plants where the targeted gene function had been initially transferred to the nucleus. This opens up an entire field of inquiry and opportunity in life sciences.

In one aspect, the invention provides an engineered meganuclease that binds and cleaves a recognition sequence comprising SEQ ID NO: 1 in a plant mitochondrial ATP synthase 1 (mtATP1) gene, wherein the engineered meganuclease comprises a first subunit and a second subunit, wherein the first subunit binds to a first recognition half-site of the recognition sequence and comprises a first hypervariable (HVR1) region, wherein the second subunit binds to a second recognition half-site of the recognition sequence and comprises a second hypervariable (HVR2) region, wherein the HVR1 region comprises an amino acid sequence having at least 80%, 85%, 90%, 91%, 92%, 93%, 94%, 95%, 96%, 97%, 98%, 99% or more sequence identity to an amino acid sequence corresponding to residues 24-79 of SEQ ID NO: 3, and wherein the HVR2 region comprises an amino acid sequence having at least 80%, 85%, 90%, 91%, 92%, 93%, 94%, 95%, 96%, 97%, 98%, 99% or more sequence identity to an amino acid sequence corresponding to residues 215-270 of SEQ ID NO: 3.

In some embodiments, the engineered meganuclease is a mitochondria-targeted engineered meganuclease (MTEM) that comprises the engineered meganuclease attached to a mitochondrial transit peptide (MTP).

In some embodiments, the HVR1 region comprises one or more residues corresponding to residues 24, 26, 28, 30, 32, 33, 38, 40, 42, 44, 46, 68, 70, 75, and 77 of SEQ ID NO: 3. In some embodiments, the HVR1 region comprises residues corresponding to residues 24, 26, 28, 30, 32, 33, 38, 40, 42, 44, 46, 68, 70, 75, and 77 of SEQ ID NO: 3. In some embodiments, the HVR1 region comprises Y, R, K, or D at a residue corresponding to residue 66 of SEQ ID NO: 3. In some embodiments, the HVR1 region comprises residues 24-79 of SEQ ID NO: 3 with up to 1, 2, 3, 4, 5, 6, 7, 8, 9, 10, or 11 amino acid substitutions. In some embodiments, the HVR1 region comprises residues 24-79 of SEQ ID NO: 3.

In some embodiments, the first subunit comprises an amino acid sequence having at least 80%, 85%, 90%, 91%, 92%, 93%, 94%, 95%, 96%, 97%, 98%, 99% or more sequence identity to residues 7-153 of SEQ ID NO: 3. In some embodiments, the first subunit comprises a residue corresponding to residue 80 of SEQ ID NO: 3. In some embodiments, the first subunit comprises G, S, or A at a residue corresponding to residue 19 of SEQ ID NO: 3. In some embodiments, the first subunit comprises E, Q, or K at a residue corresponding to residue 80 of SEQ ID NO: 3. In some embodiments, the first subunit comprises residues 7-153 of SEQ ID NO: 3 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In some embodiments, the first subunit comprises residues 7-153 of SEQ ID NO: 3.

In some embodiments, the HVR2 region comprises one or more residues corresponding to residues 215, 217, 219, 221, 223, 224, 229, 231, 233, 235, 237, 259, 261, 266, and 268 of SEQ ID NO: 3. In some embodiments, the HVR2 region comprises residues corresponding to residues 215, 217, 219, 221, 223, 224, 229, 231, 233, 235, 237, 259, 261, 266, and 268 of SEQ ID NO: 3. In some embodiments, the HVR2 region comprises Y, R, K, or D at a residue corresponding to residue 257 of SEQ ID NO: 3. In some embodiments, the HVR2 region comprises residues 215-270 of SEQ ID NO: 3 with up to 1, 2, 3, 4, 5, 6, 7, 8, 9, 10, or 11 amino acid substitutions. In some embodiments, the HVR2 region comprises residues 215-270 of SEQ ID NO: 3.

In some embodiments, the second subunit comprises an amino acid sequence having at least 80%, 85%, 90%, 91%, 92%, 93%, 94%, 95%, 96%, 97%, 98%, 99% or more sequence identity to residues 198-344 of SEQ ID NO: 3. In some embodiments, the second subunit comprises a residue corresponding to residue 210 of SEQ ID NO: 3. In some embodiments, the second subunit comprises a residue corresponding to residue 271 of SEQ ID NO: 3. In some embodiments, the second subunit comprises G, S, or A at a residue corresponding to residue 210 of SEQ ID NO: 3. In some embodiments, the second subunit comprises E, Q, or K at a residue corresponding to residue 271 of SEQ ID NO: 3. In some embodiments, the second subunit comprises residues 198-344 of SEQ ID NO: 3 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In some embodiments, the second subunit comprises residues 198-344 of SEQ ID NO: 3.

In some embodiments, the engineered meganuclease is a single-chain meganuclease comprising a linker, the linker covalently joins the first subunit and the second subunit. In some embodiments, the engineered meganuclease comprises an amino acid sequence having at least 80%, 85%, 90%, 91%, 92%, 93%, 94%, 95%, 96%, 97%, 98%, 99% or more sequence identity to SEQ ID NO: 3. In some embodiments, the engineered meganuclease comprises an amino acid sequence of SEQ ID NO: 3. In some embodiments, the engineered meganuclease is encoded by a nucleic sequence having at least 80%, 85%, 90%, 91%, 92%, 93%, 94%, 95%, 96%, 97%, 98%, 99% or more sequence identity to a nucleic acid sequence of SEQ ID NO: 4. In some embodiments, the engineered meganuclease is encoded by a nucleic acid sequence of SEQ ID NO: 4.

In another aspect, the invention provides an engineered meganuclease that binds and cleaves a recognition sequence comprising SEQ ID NO: 2 in a plant mtATP1 gene, wherein the engineered meganuclease comprises a first subunit and a second subunit, wherein the first subunit binds to a first recognition half-site of the recognition sequence and comprises a first hypervariable (HVR1) region, wherein the second subunit binds to a second recognition half-site of the recognition sequence and comprises a second hypervariable (HVR2) region, wherein the HVR1 region comprises an amino acid sequence having at least 80%, 85%, 90%, 91%, 92%, 93%, 94%, 95%, 96%, 97%, 98%, 99% or more sequence identity to an amino acid sequence corresponding to residues 24-79 of SEQ ID NO: 5, and wherein the HVR2 region comprises an amino acid sequence having at least 80%, 85%, 90%, 91%, 92%, 93%, 94%, 95%, 96%, 97%, 98%, 99% or more sequence identity to an amino acid sequence corresponding to residues 215-270 of SEQ ID NO: 5.

In some embodiments, the engineered meganuclease is an MTEM that comprises the engineered meganuclease attached to an MTP.

In some embodiments, the HVR1 region comprises an amino acid sequence having at least 80%, 85%, 90%, 91%, 92%, 93%, 94%, 95%, 96%, 97%, 98%, 99% or more sequence identity to residues 24-79 of SEQ ID NO: 5. In some embodiments, the HVR1 region comprises one or more residues corresponding to residues 24, 26, 28, 30, 32, 33, 38, 40, 42, 44, 46, 68, 70, 75, and 77 of SEQ ID NO: 5. In some embodiments, the HVR1 region comprises residues corresponding to residues 24, 26, 28, 30, 32, 33, 38, 40, 42, 44, 46, 68, 70, 75, and 77 of SEQ ID NO: 5. In some embodiments, the HVR1 region comprises Y, R, K, or D at a residue corresponding to residue 66 of SEQ ID NO: 5. In some embodiments, the HVR1 region comprises residues 24-79 of SEQ ID NO: 5 with up to 1, 2, 3, 4, 5, 6, 7, 8, 9, 10, or 11 amino acid substitutions. In some embodiments, the HVR1 region comprises residues 24-79 of SEQ ID NO: 5.

In some embodiments, the first subunit comprises an amino acid sequence having at least 80%, 85%, 90%, 91%, 92%, 93%, 94%, 95%, 96%, 97%, 98%, 99% or more sequence identity to residues 7-153 of SEQ ID NO: 5. In some embodiments, the first subunit comprises a residue corresponding to residue 80 of SEQ ID NO: 5. In some embodiments, the first subunit comprises G, S, or A at a residue corresponding to residue 19 of SEQ ID NO: 5. In some embodiments, the first subunit comprises E, Q, or K at a residue corresponding to residue 80 of SEQ ID NO: 5. In some embodiments, the first subunit comprises residues 7-153 of SEQ ID NO: 5 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In some embodiments, the first subunit comprises residues 7-153 of SEQ ID NO: 5.

In some embodiments, the HVR2 region comprises an amino acid sequence having at least 80%, 85%, 90%, 91%, 92%, 93%, 94%, 95%, 96%, 97%, 98%, 99% or more sequence identity to residues 215-270 of SEQ ID NO: 5. In some embodiments, the HVR2 region comprises one or more residues corresponding to residues 215, 217, 219, 221, 223, 224, 229, 231, 233, 235, 237, 259, 261, 266, and 268 of SEQ ID NO: 5. In some embodiments, the HVR2 region comprises residues corresponding to residues 215, 217, 219, 221, 223, 224, 229, 231, 233, 235, 237, 259, 261, 266, and 268 of SEQ ID NO: 5. In some embodiments, the HVR2 region comprises Y, R, K, or D at a residue corresponding to residue 257 of SEQ ID NO: 5. In some embodiments, the HVR2 region comprises residues 215-270 of SEQ ID NO: 5 with up to 1, 2, 3, 4, 5, 6, 7, 8, 9, 10, or 11 amino acid substitutions. In some embodiments, the HVR2 region comprises residues 215-270 of SEQ ID NO: 5.

In some embodiments, the second subunit comprises an amino acid sequence having at least 80%, 85%, 90%, 91%, 92%, 93%, 94%, 95%, 96%, 97%, 98%, 99% or more sequence identity to residues 198-344 of SEQ ID NO: 5. In some embodiments, the second subunit comprises a residue corresponding to residue 210 of SEQ ID NO: 5. In some embodiments, the second subunit comprises a residue corresponding to residue 271 of SEQ ID NO: 5. In some embodiments, the second subunit comprises G, S, or A at a residue corresponding to residue 210 of SEQ ID NO: 5. In some embodiments, the second subunit comprises E, Q, or K at a residue corresponding to residue 271 of SEQ ID NO: 5. In some embodiments, the second subunit comprises residues 198-344 of SEQ ID NO: 5 with up to 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions. In some embodiments, the second subunit comprises residues 198-344 of SEQ ID NO: 5.

In some embodiments, the engineered meganuclease is a single-chain meganuclease comprising a linker, wherein the linker covalently joins the first subunit and the second subunit. In some embodiments, the engineered meganuclease comprises an amino acid sequence having at least 80%, 85%, 90%, 91%, 92%, 93%, 94%, 95%, 96%, 97%, 98%, 99% or more sequence identity to SEQ ID NO: 5. In some embodiments, the engineered meganuclease comprises an amino acid sequence of SEQ ID NO: 5. In some embodiments, the engineered meganuclease is encoded by a nucleic sequence having at least 80%, 85%, 90%, 91%, 92%, 93%, 94%, 95%, 96%, 97%, 98%, 99% or more sequence identity to a nucleic acid sequence of SEQ ID NO: 6. In some embodiments, the engineered meganuclease is encoded by a nucleic acid sequence of SEQ ID NO: 6.

In some embodiments, the MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the MTP comprises an amino acid sequence set forth in any one of SEQ ID NOs: 7-11. In some embodiments, the MTP is attached to the C-terminus of the engineered meganuclease. In some embodiments, the MTP is attached to the N-terminus of the engineered meganuclease. In some embodiments, the MTP is fused to the engineered meganuclease. In some embodiments, the MTP is attached to the engineered meganuclease by a polypeptide linker. In some embodiments, the engineered meganuclease is attached to a first MTP and a second MTP. In some embodiments, the first MTP and/or the second MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the first MTP and/or the second MTP comprises an amino acid sequence set forth in any one of SEQ ID NOs: 7-11. In some embodiments, the first MTP and the second MTP are identical. In some embodiments, the first MTP and the second MTP are not identical. In some embodiments, the first MTP and/or the second MTP is fused to the engineered meganuclease. In some embodiments, the first MTP and/or the second MTP is attached to the engineered meganuclease by a polypeptide linker.

In some embodiments, the MTEM is attached to a nuclear export sequence (NES). In some embodiments, the NES comprises an amino acid sequence having at least 80%, 85%, 90%, 91%, 92%, 93%, 94%, 95%, 96%, 97%, 98%, 99% or more sequence identity to a sequence set forth in SEQ ID NO: 12 or 13. In some embodiments, the NES comprises an amino acid sequence set forth in SEQ ID NO: 12 or 13. In some embodiments, the NES is attached at the N-terminus of the MTEM. In some embodiments, the NES is attached at the C-terminus of the MTEM. In some embodiments, the NES is fused to the MTEM. In some embodiments, the NES is attached to the MTEM by a polypeptide linker. In some embodiments, the MTEM comprises a first NES and a second NES. In some embodiments, the first NES is attached at the N-terminus of the MTEM, and the second NES is attached at the C-terminus of the MTEM. In some embodiments, the first NES and/or the second NES comprises an amino acid sequence having at least 80%, 85%, 90%, 91%, 92%, 93%, 94%, 95%, 96%, 97%, 98%, 99% or more sequence identity to a sequence set forth in SEQ ID NO: 12 or 13. In some embodiments, the first NES and/or the second NES comprises an amino acid sequence set forth in SEQ ID NO: 12 or 13. In some embodiments, the first NES and the second NES are identical. In some embodiments, the first NES and the second NES are not identical. In some embodiments, the first NES and/or the second NES is fused to the MTEM. In some embodiments, the first NES and/or the second NES is attached to the MTEM by a polypeptide linker.

In another aspect, the invention provides a polynucleotide comprising a nucleic acid sequence encoding an engineered meganuclease or MTEM described herein. In some embodiments, the polynucleotide is an mRNA. In some embodiments, the polynucleotide further comprises a nucleic acid sequence encoding a selectable marker. In some embodiments, the selectable marker is an antibiotic resistance gene.

In another aspect, the invention provides an expression cassette comprising any polynucleotide described herein. In some embodiments, the polynucleotide comprises a promoter that is operably linked to the nucleic acid sequence encoding the MTEM or engineered meganuclease. In some embodiments, the promoter is a tissue-specific promoter. In some embodiments, the promoter is active in a plant cell. In some embodiments, the promoter is active in a flower of a plant. In some embodiments, the promoter is a flower-specific promoter or a flower-preferred promoter. In some embodiments, the promoter is an anther-specific promoter, an anther-preferred promoter, a pollen-specific promoter, or a pollen-preferred promoter. In some embodiments, the promoter is a constitutively active promoter.

In another aspect, the invention provides an expression cassette comprising a polynucleotide comprising a nucleic acid sequence encoding any MTEN described herein, wherein said MTEN comprises an engineered nuclease attached to an MTP described herein, and wherein said MTEN binds and cleaves a recognition sequence in a male-essential plant mitochondrial gene described herein. In some embodiments, the MTP is attached to the MTEN in any manner of attachment described herein (e.g., fusion or use of a polypeptide linker). In some embodiments, the MTEN is attached to any NES described herein, using any manner of attachment of an NES described herein. In some embodiments, the expression cassette comprises any promoter described herein, operably linked to the nucleic acid sequence encoding the MTEN.

In another aspect, the invention provides an expression cassette comprising a polynucleotide comprising a nucleic acid sequence encoding a mitochondria-targeting engineered nuclease (MTEN) described herein, wherein the MTEN comprises an engineered nuclease attached to an MTP, and wherein the MTEN binds and cleaves a first recognition sequence in a male-essential plant mitochondrial gene. In some embodiments, the MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the MTP comprises an amino acid sequence set forth in any one of SEQ ID NOs: 7-11. In some embodiments, the male-essential plant mitochondrial gene is an mtATP gene. In some embodiments, the male-essential plant mitochondrial gene is an mtATP1 gene. In some embodiments, the engineered nuclease is an engineered meganuclease, a CRISPR system nuclease, a compact TALEN, or a megaTAL. In some embodiments, the MTP is attached to the C-terminus of the engineered nuclease. In some embodiments, the MTP is attached to the N-terminus of the engineered nuclease. In some embodiments, the MTP is fused to the engineered nuclease. In some embodiments, the MTP is attached to the engineered nuclease by a polypeptide linker. In some embodiments, the engineered nuclease is attached to a first MTP and a second MTP, wherein at least one of the first MTP and the second MTP is an MTP described herein. In some embodiments, the first MTP and the second MTP are identical. In some embodiments, the first MTP and the second MTP are not identical. In some embodiments, the first MTP and/or the second MTP is fused to the engineered nuclease. In some embodiments, the first MTP and/or the second MTP is attached to the engineered nuclease by a polypeptide linker. In some embodiments, the engineered nuclease is a zinc finger nuclease or a TALEN. In some embodiments, the MTP is attached to the N-terminus of the engineered nuclease. In some embodiments, the MTP is fused to the engineered nuclease. In some embodiments, the MTP is attached to the engineered nuclease by a polypeptide linker. In some embodiments, the recognition sequence comprises SEQ ID NO: 1. In some embodiments, the recognition sequence comprises SEQ ID NO: 2. In some embodiments, the expression cassette comprises a promoter that is operably linked to the nucleic acid sequence encoding the MTEN. In some embodiments, the MTEN is an MTEM described herein that binds and cleaves a recognition sequence comprising SEQ ID NO: 1 in a plant mtATP1 gene. In some embodiments, the MTEN is an MTEM described herein that binds and cleaves a recognition sequence comprising SEQ ID NO: 2 in a plant mtATP1 gene.

In some embodiments, the MTEN is attached to a nuclear export sequence (NES). In some embodiments, the NES comprises an amino acid sequence having at least 80%, 85%, 90%, 91%, 92%, 93%, 94%, 95%, 96%, 97%, 98%, 99% or more sequence identity to a sequence set forth in SEQ ID NO: 12 or 13. In some embodiments, the NES comprises an amino acid sequence set forth in SEQ ID NO: 12 or 13. In some embodiments, the NES is attached at the N-terminus of the MTEN. In some embodiments, the NES is attached at the C-terminus of the MTEN. In some embodiments, the NES is fused to the MTEN. In some embodiments, the NES is attached to the MTEN by a polypeptide linker. In some embodiments, the MTEN comprises a first NES and a second NES. In some embodiments, the first NES is attached at the N-terminus of the MTEN, and wherein the second NES is attached at the C-terminus of the MTEN. In some embodiments, the first NES and/or the second NES comprises an amino acid sequence having at least 80%, 85%, 90%, 91%, 92%, 93%, 94%, 95%, 96%, 97%, 98%, 99% or more sequence identity to a sequence set forth in SEQ ID NO: 12 or 13. In some embodiments, the first NES and/or the second NES comprises an amino acid sequence set forth in SEQ ID NO: 12 or 13. In some embodiments, the first NES and the second NES are identical. In some embodiments, the first NES and the second NES are not identical. In some embodiments, the first NES and/or the second NES is fused to the MTEN. In some embodiments, the first NES and/or the second NES is attached to the MTEN by a polypeptide linker. In some embodiments, the first MTEN and the second MTEN are capable of generating cleavage sites having complementary overhangs. In some embodiments, the expression cassette comprises a promoter that is operably linked to the nucleic acid sequence encoding the MTEN or engineered meganuclease. In some embodiments, the promoter is a tissue-specific promoter. In some embodiments, the promoter is active in a plant cell. In some embodiments, the promoter is active in a flower of a plant. In some embodiments, the promoter is a flower-specific promoter or a flower-preferred promoter. In some embodiments, the promoter is an anther-specific promoter, an anther-preferred promoter, a pollen-specific promoter, or a pollen-preferred promoter. In some embodiments, the promoter is a constitutively active promoter and/or a ubiquitous promoter.

In another aspect, the expression cassette comprises a) a first polynucleotide comprising a nucleic acid sequence encoding a first MTEN; and (b) a second polynucleotide comprising a nucleic acid sequence encoding a second MTEN; wherein the first MTEN and the second MTEN each comprise an engineered nuclease attached to an MTP, wherein the first MTEN binds and cleaves a first recognition sequence in the male-essential plant mitochondrial gene, and wherein the second MTEN binds and cleaves a second recognition sequence in the male-essential plant mitochondrial gene. In some embodiments, the first MTEN and the second MTEN are capable of generating cleavage sites having complementary overhangs. In some embodiments, the first recognition sequence and the second recognition sequence are less than about 1500, about 1400, about 1300, about 1200, about 1100, about 1000, about 900, about 800, about 700, about 600, about 500, about 400, about 300, about 200, about 100, or about 50 basepairs apart in the male-essential plant mitochondrial gene. In some embodiments, the first MTEN and/or the second MTEN is an MTEM. In some embodiments, the first recognition sequence and the second recognition sequence comprise identical 4 basepair center sequences. In some embodiments, the first recognition sequence comprises SEQ ID NO: 1. In some embodiments, the second recognition sequence comprises SEQ ID NO: 2. In some embodiments, the first MTEN is an MTEM described herein that binds and cleaves a recognition sequence comprising SEQ ID NO: 1 in a plant mtATP1 gene. In some embodiments, the second MTEN is an MTEM described herein that binds and cleaves a recognition sequence comprising SEQ ID NO: 2 in a plant mtATP1 gene. In some embodiments, the expression cassette comprises a promoter that is operably linked to the nucleic acid sequence encoding the first MTEN and the nucleic acid sequence encoding the second MTEN. In some embodiments, the nucleic acid sequence encoding the first MTEN and the second nucleic acid sequence encoding the second MTEN are separated by an IRES or 2A sequence. In some embodiments, the 2A sequence is a T2A, a P2A, an E2A, or an F2A sequence. In some embodiments, the promoter is a tissue-specific promoter. In some embodiments, the promoter is active in a plant cell. In some embodiments, the promoter is active in a flower of a plant. In some embodiments, the promoter is a flower-specific promoter or a flower-preferred promoter. In some embodiments, the promoter is an anther-specific promoter, an anther-preferred promoter, a pollen-specific promoter, or a pollen-preferred promoter. In some embodiments, the promoter is a constitutively active promoter and/or a ubiquitous promoter.

In some embodiments, the expression cassette comprises a first promoter that is operably linked to the nucleic acid sequence encoding the first MTEN, and a second promoter that is operably linked to the nucleic acid sequence encoding the second MTEN. In some embodiments, the first promoter and the second promoter are identical. In some embodiments, the first promoter and the second promoter are not identical. In some embodiments, the first promoter and/or the second promoter is a tissue-specific promoter. In some embodiments, the first promoter and/or the second promoter is active in a plant cell. In some embodiments, the first promoter and/or the second promoter is active in a flower of a plant. In some embodiments, the first promoter and/or the second promoter is a flower-specific promoter or a flower-preferred promoter. In some embodiments, the first promoter and/or the second promoter is an anther-specific promoter, an anther-preferred promoter, a pollen-specific promoter, or a pollen-preferred promoter. In some embodiments, the first promoter and/or the second promoter is a constitutively active promoter and/or a ubiquitous promoter.

In another aspect, the invention provides any recombinant DNA construct described herein comprising a polynucleotide comprising any expression cassette comprising any polynucleotide comprising a nucleic acid sequence encoding any MTEM described herein.

In another aspect, the invention provides any recombinant DNA construct described herein comprising a polynucleotide comprising any expression cassette comprising a polynucleotide comprising a nucleic acid sequence encoding a mitochondria-targeting engineered nuclease (MTEN) described herein, wherein the MTEN comprises an engineered nuclease attached to an MTP, and wherein the MTEN binds and cleaves a first recognition sequence in a male-essential plant mitochondrial gene. In some embodiments, the recombinant DNA construct comprises a polynucleotide comprising (a) a first polynucleotide comprising a nucleic acid sequence encoding a first mitochondria-targeting engineered nuclease (MTEN) described herein; and (b) a second polynucleotide comprising a nucleic acid sequence encoding a second MTEN described herein; wherein the MTEN comprises an engineered meganuclease attached to an MTP described herein; wherein the first MTEN binds and cleaves a first recognition sequence in a male-essential plant mitochondrial gene, and wherein the second MTEN binds and cleaves a second recognition sequence in the male-essential plant mitochondrial gene.

In another aspect, the invention provides a bacterium comprising any recombinant DNA construct described herein comprising a polynucleotide comprising any expression cassette comprising any polynucleotide comprising a nucleic acid sequence encoding any MTEM described herein. In some embodiments, the bacterium is Agrobacterium tumefaciens.

In another aspect, the invention provides a bacterium comprising any recombinant DNA construct described herein comprising a polynucleotide comprising any expression cassette comprising a polynucleotide comprising a nucleic acid sequence encoding a mitochondria-targeting engineered nuclease (MTEN) described herein, wherein the MTEN comprises an engineered nuclease attached to an MTP, and wherein the MTEN binds and cleaves a first recognition sequence in a male-essential plant mitochondrial gene. In some embodiments, the invention provides a bacterium comprising any recombinant DNA construct described herein comprising a polynucleotide comprising any expression cassette comprising (a) a first polynucleotide comprising a nucleic acid sequence encoding a first mitochondria-targeting engineered nuclease (MTEN) described herein; and (b) a second polynucleotide comprising a nucleic acid sequence encoding a second MTEN described herein; wherein the MTEN comprises an engineered meganuclease attached to an MTP described herein; wherein the first MTEN binds and cleaves a first recognition sequence in a male-essential plant mitochondrial gene, and wherein the second MTEN binds and cleaves a second recognition sequence in the male-essential plant mitochondrial gene. In some embodiments, the bacterium is Agrobacterium tumefaciens.

In another aspect, the invention provides a recombinant virus comprising a polynucleotide comprising any expression cassette comprising any polynucleotide comprising a nucleic acid sequence encoding any MTEM described herein. In some embodiments, the recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In another aspect, the invention provides a recombinant virus comprising a polynucleotide comprising any expression cassette comprising a polynucleotide comprising a nucleic acid sequence encoding a mitochondria-targeting engineered nuclease (MTEN) described herein, wherein the MTEN comprises an engineered nuclease attached to an MTP, and wherein the MTEN binds and cleaves a first recognition sequence in a male-essential plant mitochondrial gene. In some embodiments, the invention provides a recombinant virus comprising a polynucleotide comprising any expression cassette comprising (a) a first polynucleotide comprising a nucleic acid sequence encoding a first mitochondria-targeting engineered nuclease (MTEN) described herein; and (b) a second polynucleotide comprising a nucleic acid sequence encoding a second MTEN described herein; wherein the MTEN comprises an engineered meganuclease attached to an MTP described herein; wherein the first MTEN binds and cleaves a first recognition sequence in a male-essential plant mitochondrial gene, and wherein the second MTEN binds and cleaves a second recognition sequence in the male-essential plant mitochondrial gene. In some embodiments, the recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In another aspect, the invention provides a polynucleotide comprising a sequence set forth in SEQ ID NO: 14. In some embodiments, the polynucleotide is a plant mtATP1 gene comprising a sequence set forth in SEQ ID NO: 15.

In another aspect, the invention provides a genetically-modified plant cell comprising any polynucleotide described herein.

In another aspect, the invention provides a genetically-modified plant cell comprising any expression cassette described herein.

In another aspect, the invention provides a genetically-modified plant cell comprising any recombinant DNA construct described herein.

In another aspect, the invention provides a genetically-modified plant cell comprising a modified male-essential mitochondrial gene. In some embodiments, the modified male-essential mitochondrial gene is inactivated. In some embodiments, the modified male-essential mitochondrial gene is a modified mtATP gene. In some embodiments, the modified male-essential mitochondrial gene is a modified mtATP1 gene. In some embodiments, the modified mtATP1 gene comprises a nucleic acid sequence set forth in SEQ ID NO: 14. In some embodiments, the genetically-modified plant cell is a genetically-modified tobacco cell. In some embodiments, the genetically-modified plant cell comprises a maintainer construct on a nuclear chromosome, wherein the maintainer construct comprises: (a) a copy of the male-essential mitochondrial gene which encodes a wild-type polypeptide; (b) a non-male promoter operably linked to the copy of the male-essential mitochondrial gene; and (c) a nucleic acid sequence encoding a maintainer MTP which is attached to the wild-type polypeptide. In some embodiments, the copy of the male-essential mitochondrial gene in the maintainer construct is codon-optimized for expression in the nucleus and encodes a wild-type polypeptide. In some embodiments, the copy of the male-essential mitochondrial gene in the maintainer construct encodes a wild-type polypeptide but is modified to not comprise the recognition sequence, first recognition sequence, or the second recognition sequence. In some embodiments, the maintainer MTP is attached to the N-terminus of the wild-type polypeptide. In some embodiments, the maintainer MTP is attached to the C-terminus of the wild-type polypeptide. In some embodiments, the maintainer MTP is fused to the wild-type polypeptide. In some embodiments, the maintainer MTP is attached to the wild-type polypeptide by a polypeptide linker. In some embodiments, the maintainer MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the maintainer MTP comprises an amino acid sequence set forth in any one of SEQ ID NOs: 7-11. In some embodiments, the maintainer MTP comprises an amino acid sequence set forth in SEQ ID NO: 7. In some embodiments, the non-male promoter is a weak non-male promoter. In some embodiments, the non-male promoter is a CaMV35S promoter or an enhanced CaMV35S promoter. In some embodiments, the promoter is a strong non-male promoter. In some embodiments, the strong non-male promoter is a ubiquitin promoter. In some embodiments, the genetically-modified plant cell comprises a restorer construct on a nuclear chromosome, wherein the restorer construct comprises: (a) a copy of the male-essential mitochondrial gene which encodes a wild-type polypeptide; (b) a ubiquitous promoter operably linked to the copy of the male-essential mitochondrial gene; and (c) a nucleic acid sequence encoding a restorer MTP which is attached to the wild-type polypeptide. In some embodiments, the copy of the male-essential mitochondrial gene in the restorer construct is codon-optimized for expression in the nucleus and encodes a wild-type polypeptide. In some embodiments, the copy of the male-essential mitochondrial gene in the restorer construct encodes a wild-type polypeptide but is modified to not comprise the first recognition sequence or the second recognition sequence. In some embodiments, the restorer MTP is attached to the N-terminus of the wild-type polypeptide. In some embodiments, the restorer MTP is attached to the C-terminus of the wild-type polypeptide. In some embodiments, the restorer MTP is fused to the wild-type polypeptide. In some embodiments, the restorer MTP is attached to the wild-type polypeptide by a polypeptide linker. In some embodiments, the restorer MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the restorer MTP comprises an amino acid sequence set forth in any one of SEQ ID NOs: 7-11. In some embodiments, the restorer MTP comprises an amino acid sequence set forth in SEQ ID NO: 7. In some embodiments, the ubiquitous promoter is a weak ubiquitous promoter. In some embodiments, the ubiquitous promoter is an mtATP promoter. In some embodiments, the ubiquitous promoter is a β-ATP promoter. In some embodiments, the ubiquitous promoter is a strong ubiquitous promoter. In some embodiments, the strong ubiquitous promoter is a ubiquitin promoter.

In another aspect, the invention provides a plant or plant part comprising the genetically-modified plant cell described herein. In some embodiments, the plant part is a seed comprising the genetically-modified plant cell.

In another aspect, the invention provides a maintainer plant cell that comprises a maintainer construct on a nuclear chromosome, wherein said maintainer construct comprises: (a) a copy of a male-essential mitochondrial gene which encodes a wild-type polypeptide; (b) a non-male promoter operably linked to the copy of the male-essential mitochondrial gene; and (c) a nucleic acid sequence encoding a maintainer MTP which is attached to the wild-type polypeptide. In some embodiments, the copy of the male-essential mitochondrial gene in the maintainer construct is codon-optimized for expression in the nucleus and encodes a wild-type polypeptide. In some embodiments, the copy of the male-essential mitochondrial gene in the maintainer construct encodes a wild-type polypeptide but is modified to not comprise the recognition sequence, first recognition sequence, or the second recognition sequence. In some embodiments, the maintainer MTP is attached to the N-terminus of the wild-type polypeptide. In some embodiments, the maintainer MTP is attached to the C-terminus of the wild-type polypeptide. In some embodiments, the maintainer MTP is fused to the wild-type polypeptide. In some embodiments, the maintainer MTP is attached to the wild-type polypeptide by a polypeptide linker. In some embodiments, the maintainer MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the maintainer MTP comprises an amino acid sequence set forth in any one of SEQ ID NOs: 7-11. In some embodiments, the maintainer MTP comprises an amino acid sequence set forth in SEQ ID NO: 7. In some embodiments, the non-male promoter is a weak non-male promoter. In some embodiments, the non-male promoter is a CaMV35S promoter or an enhanced CaMV35S promoter. In some embodiments, the promoter is a strong non-male promoter. In some embodiments, the strong non-male promoter expresses said male-essential mitochondrial gene in comparable levels to the levels of the male-essential mitochondrial gene expressed from the mitochondrial gene. In some embodiments, the invention provides a maintainer plant or maintainer plant part comprising the maintainer plant cell, such as a maintainer plant cell with a strong non-male promoter. In some embodiments, the invention provides a maintainer plant or maintainer plant part comprising the maintainer plant cell, such as a maintainer plant cell with a weak non-male promoter. In some embodiments, the invention comprises a seed comprising the maintainer plant cell.

In another aspect, the invention provides a restorer plant cell that comprises a restorer construct on a nuclear chromosome, wherein said restorer construct comprises: (a) a copy of a male-essential mitochondrial gene which encodes a wild-type polypeptide; (b) a ubiquitous promoter operably linked to the copy of the male-essential mitochondrial gene; and (c) a nucleic acid sequence encoding a restorer MTP which is attached to the wild-type polypeptide. In some embodiments, the copy of the male-essential mitochondrial gene in the restorer construct is codon-optimized for expression in the nucleus and encodes a wild-type polypeptide. In some embodiments, the copy of the male-essential mitochondrial gene in the restorer construct encodes a wild-type polypeptide but is modified to not comprise the first recognition sequence or the second recognition sequence. In some embodiments, the restorer MTP is attached to the N-terminus of the wild-type polypeptide. In some embodiments, the restorer MTP is attached to the C-terminus of the wild-type polypeptide. In some embodiments, the restorer MTP is fused to the wild-type polypeptide. In some embodiments, the restorer MTP is attached to the wild-type polypeptide by a polypeptide linker. In some embodiments, the restorer MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the restorer MTP comprises an amino acid sequence set forth in any one of SEQ ID NOs: 7-11. In some embodiments, the restorer MTP comprises an amino acid sequence set forth in SEQ ID NO: 7. In some embodiments, the ubiquitous promoter is a weak ubiquitous promoter. In some embodiments, the ubiquitous promoter is an mtATP promoter. In some embodiments, the ubiquitous promoter is a β-ATP promoter. In some embodiments, the ubiquitous promoter is a strong ubiquitous promoter. In some embodiments, the strong ubiquitous promoter is a ubiquitin promoter. In some embodiments, the invention provides a restorer plant or restorer plant part comprising the restorer plant cell, such as a restorer plant cell with a strong ubiquitous promoter. In some embodiments, the invention provides a restorer plant or restorer plant part comprising the restorer plant cell, such as a restorer plant cell with a weak ubiquitous promoter. In some embodiments, the invention comprises a seed comprising the restorer plant cell.

In another aspect, the invention provides a method for producing a genetically-modified plant cell, the method comprising introducing into a plant cell (a) a polynucleotide comprising a nucleic acid sequence encoding any MTEM described herein, wherein the MTEM is expressed in the plant cell; or (b) any MTEM described herein; wherein the MTEM produces a cleavage site at the recognition sequence in the mtATP1 gene in mitochondrial genomes. In some embodiments, the cleavage site is repaired, such that the recognition sequence comprises an insertion or deletion. In some embodiments, the insertion or deletion inactivates said mtATP1 gene. In some embodiments, the mitochondrial genomes comprising the recognition sequence are degraded in the genetically-modified plant cell. In some embodiments, the polynucleotide is an mRNA. In some embodiments, the mRNA is any mRNA described herein. In some embodiments, the polynucleotide is a recombinant DNA construct. In some embodiments, the polynucleotide is a recombinant DNA construct described herein. In some embodiments, the polynucleotide is introduced into the plant cell by a recombinant virus. In some embodiments, the recombinant virus is a recombinant virus of described herein. In some embodiments, the polynucleotide is introduced into the plant cell by a bacterium of described herein. In some embodiments, the polynucleotide is introduced into the plant cell by Agrobacterium-mediated transformation, biolistic transformation, by microinjection, or by electroporation. In some embodiments, the polynucleotide comprises an expression cassette described herein.

In another aspect, the invention provides a method for producing a genetically-modified plant cell, the method comprising introducing into a plant cell a polynucleotide comprising a nucleic acid sequence encoding any MTEN described herein, wherein the MTEN comprises an engineered nuclease attached to an MTP described herein, wherein the MTEN binds and cleaves a recognition sequence in a male-essential plant mitochondrial gene described herein to produce a cleavage site. In some embodiments, the cleavage site is repaired, such that said recognition sequence comprises an insertion or deletion. In some embodiments, the insertion or deletion inactivates the male-essential plant mitochondrial gene. In some embodiments, the MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the MTP comprises an amino acid sequence set forth in any one of SEQ ID NOs: 7-11. In some embodiments, the male-essential plant mitochondrial gene is an mtATP gene. In some embodiments, the male-essential plant mitochondrial gene is an mtATP1 gene. In some embodiments, the engineered nuclease is an engineered meganuclease, a CRISPR system nuclease, a compact TALEN, or a megaTAL. In some embodiments, the MTP is attached to the C-terminus of the engineered nuclease. In some embodiments, the MTP is attached to the N-terminus of the engineered nuclease. In some embodiments, the MTP is fused to the engineered nuclease. In some embodiments, the MTP is attached to the engineered nuclease by a polypeptide linker. In some embodiments, the engineered nuclease is attached to a first MTP and a second MTP, wherein at least one of the first MTP and the second MTP is an MTP described herein. In some embodiments, the first MTP and the second MTP are identical. In some embodiments, the first MTP and the second MTP are not identical. In some embodiments, the first MTP and/or the second MTP is fused to the engineered nuclease. In some embodiments, the first MTP and/or the second MTP is attached to the engineered nuclease by a polypeptide linker. In some embodiments, the engineered nuclease is a zinc finger nuclease or a TALEN. In some embodiments, the MTP is attached to the N-terminus of the engineered nuclease. In some embodiments, the MTP is fused to the engineered nuclease. In some embodiments, the MTP is attached to the engineered nuclease by a polypeptide linker. In some embodiments, the recognition sequence comprises SEQ ID NO: 1. In some embodiments, the recognition sequence comprises SEQ ID NO: 2. In some embodiments, the polynucleotide comprises a promoter that is operably linked to the nucleic acid sequence encoding the MTEN. In some embodiments, the MTEN is an MTEM described herein that binds and cleaves a recognition sequence comprising SEQ ID NO: 1 in a plant mtATP1 gene. In some embodiments, the MTEN is an MTEM described herein that binds and cleaves a recognition sequence comprising SEQ ID NO: 2 in a plant mtATP1 gene. In some embodiments, the promoter is a tissue-specific promoter. In some embodiments, the promoter is active in a plant cell. In some embodiments, the promoter is active in a flower of a plant. In some embodiments, the promoter is a flower-specific promoter or a flower-preferred promoter. In some embodiments, the promoter is an anther-specific promoter, an anther-preferred promoter, a pollen-specific promoter, or a pollen-preferred promoter. In some embodiments, the promoter is a constitutively active promoter and/or a ubiquitous promoter. In some embodiments, the first polynucleotide and/or the second polynucleotide is introduced into the plant cell by a recombinant virus. In some embodiments, the recombinant virus is any recombinant virus described herein. In some embodiments, the first polynucleotide and/or the second polynucleotide is introduced into the plant cell by any bacterium described herein. In some embodiments, the first polynucleotide and/or the second polynucleotide is introduced into the plant cell by Agrobacterium-mediated transformation, biolistic transformation, by microinjection, or by electroporation. In some embodiments, an expression cassette is introduced into the plant cell that comprises the first polynucleotide and the second polynucleotide. In some embodiments, the expression cassette is any expression cassette described herein. In some embodiments, the expression cassette is introduced into the plant cell using a recombinant DNA construct. In some embodiments, the expression cassette is introduced into the plant cell using a recombinant virus. In some embodiments, the recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV). In some embodiments, the expression cassette is introduced into the plant cell using a bacterium. In some embodiments, the bacterium is Agrobacterium tumefaciens. In some embodiments, the expression cassette is introduced into the plant cell by Agrobacterium-mediated transformation, biolistic transformation, by microinjection, or by electroporation.

In some embodiments, the MTP is attached to the MTEN in any manner of attachment described herein. In some embodiments, the MTEN is attached to any NES sequence described herein, in any manner of attachment of an NES described herein. In some embodiments, the expression cassette comprises any promoter described herein, operably linked to the nucleic acid sequence encoding the MTEN.

In another aspect, the invention provides a method for producing a genetically-modified plant cell, the method comprising introducing into a plant cell: (a) a first polynucleotide comprising a nucleic acid sequence encoding a first MTEN; and (b) a second polynucleotide comprising a nucleic acid sequence encoding a second MTEN; wherein the first MTEN and the second MTEN each comprise an engineered nuclease attached to an MTP; wherein the first MTEN binds and cleaves a first recognition sequence in a male-essential plant mitochondrial gene to generate a first cleavage site, and wherein the second MTEN binds and cleaves a second recognition sequence in the male-essential plant mitochondrial gene to generate a second cleavage site.

In some embodiments, the first cleavage site and said second cleavage site are repaired, such that the first recognition sequence and the second recognition sequence comprise an insertion or deletion. In some embodiments, the insertion or deletion inactivates the male-essential plant mitochondrial gene. In some embodiments, the intervening genomic sequence between the first cleavage site and the second cleavage site is removed. In certain embodiments, the first cleavage site and the second cleavage site ligate to one another to anneal the mitochondrial genome to generate a modified male-essential plant mitochondrial gene.

In some embodiments, the MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the MTP comprises an amino acid sequence set forth in any one of SEQ ID NOs: 7-11. In some embodiments, the male-essential plant mitochondrial gene is an mtATP gene. In some embodiments, the male-essential plant mitochondrial gene is an mtATP1 gene. In some embodiments, the engineered nuclease is an engineered meganuclease, a CRISPR system nuclease, a compact TALEN, or a megaTAL. In some embodiments, the MTP is attached to the C-terminus of the engineered nuclease. In some embodiments, the MTP is attached to the N-terminus of the engineered nuclease. In some embodiments, the MTP is fused to the engineered nuclease. In some embodiments, the MTP is attached to the engineered nuclease by a polypeptide linker. In some embodiments, the engineered nuclease is attached to a first MTP and a second MTP, wherein at least one of the first MTP and the second MTP is an MTP described herein. In some embodiments, the first MTP and the second MTP are identical. In some embodiments, the first MTP and the second MTP are not identical. In some embodiments, the first MTP and/or the second MTP is fused to the engineered nuclease. In some embodiments, the first MTP and/or the second MTP is attached to the engineered nuclease by a polypeptide linker. In some embodiments, the engineered nuclease is a zinc finger nuclease or a TALEN. In some embodiments, the MTP is attached to the N-terminus of the engineered nuclease. In some embodiments, the MTP is fused to the engineered nuclease. In some embodiments, the MTP is attached to the engineered nuclease by a polypeptide linker. In some embodiments, the MTEN is attached to a nuclear export sequence (NES). In some embodiments, the NES comprises an amino acid sequence having at least 80%, 85%, 90%, 91%, 92%, 93%, 94%, 95%, 96%, 97%, 98%, 99% or more sequence identity to a sequence set forth in SEQ ID NO: 12 or 13. In some embodiments, the NES comprises an amino acid sequence set forth in SEQ ID NO: 12 or 13. In some embodiments, the NES is attached at the N-terminus of the MTEN. In some embodiments, the NES is attached at the C-terminus of the MTEN. In some embodiments, the NES is fused to the MTEN. In some embodiments, the NES is attached to the MTEN by a polypeptide linker. In some embodiments, the MTEN comprises a first NES and a second NES. In some embodiments, the first NES is attached at the N-terminus of the MTEN, and wherein the second NES is attached at the C-terminus of the MTEN. In some embodiments, the first NES and/or the second NES comprises an amino acid sequence having at least 80%, 85%, 90%, 91%, 92%, 93%, 94%, 95%, 96%, 97%, 98%, 99% or more sequence identity to a sequence set forth in SEQ ID NO: 12 or 13. In some embodiments, the first NES and/or the second NES comprises an amino acid sequence set forth in SEQ ID NO: 12 or 13. In some embodiments, the first NES and the second NES are identical. In some embodiments, the first NES and the second NES are not identical. In some embodiments, the first NES and/or the second NES is fused to the MTEN. In some embodiments, the first NES and/or the second NES is attached to the MTEN by a polypeptide linker. In some embodiments, the first MTEN and the second MTEN are capable of generating cleavage sites having complementary overhangs. In some embodiments, the first recognition sequence and the second recognition sequence are less than about 1500, about 1400, about 1300, about 1200, about 1100, about 1000, about 900, about 800, about 700, about 600, about 500, about 400, about 300, about 200, about 100, or about 50 basepairs apart in the male-essential plant mitochondrial gene. In some embodiments, the first MTEN and/or the second MTEN is an MTEM. In some embodiments, the first recognition sequence and the second recognition sequence comprise identical 4 basepair center sequences. In some embodiments, the first recognition sequence comprises SEQ ID NO: 1. In some embodiments, the second recognition sequence comprises SEQ ID NO: 2. In some embodiments, the first MTEN is an MTEM described herein that binds and cleaves a recognition sequence comprising SEQ ID NO: 1. In some embodiments, the second MTEN is an MTEM described herein that binds and cleaves a recognition sequence comprising SEQ ID NO: 2. In some embodiments, the first MTEN is operably linked to a first promoter, and the second MTEN is operably linked to a second promoter. In some embodiments, the first promoter and the second promoter are identical. In some embodiments, the first promoter and the second promoter are not identical. In some embodiments, the first promoter and/or the second promoter is a tissue-specific promoter. In some embodiments, the first promoter and/or the second promoter is active in a plant cell. In some embodiments, the first promoter and/or the second promoter is active in a flower of a plant. In some embodiments, the first promoter and/or the second promoter is a flower-specific promoter or a flower-preferred promoter. In some embodiments, the first promoter and/or the second promoter is an anther-specific promoter, an anther-preferred promoter, a pollen-specific promoter, or a pollen-preferred promoter. In some embodiments, the first promoter and/or the second promoter is a constitutively active promoter and/or a ubiquitous promoter. In some embodiments, the first polynucleotide and/or the second polynucleotide is an mRNA. In some embodiments, the first polynucleotide and/or the second polynucleotide is any mRNA described herein. In some embodiments, the first polynucleotide and/or the second polynucleotide is a recombinant DNA construct. In some embodiments, the first polynucleotide and/or the second polynucleotide is any recombinant DNA construct described herein. In some embodiments, the first polynucleotide and/or the second polynucleotide is introduced into the plant cell by a recombinant virus. In some embodiments, the recombinant virus is any recombinant virus described herein. In some embodiments, the first polynucleotide and/or the second polynucleotide is introduced into the plant cell by any bacterium described herein. In some embodiments, the first polynucleotide and/or the second polynucleotide is introduced into the plant cell by Agrobacterium-mediated transformation, biolistic transformation, by microinjection, or by electroporation. In some embodiments, an expression cassette is introduced into the plant cell that comprises the first polynucleotide and the second polynucleotide. In some embodiments, the expression cassette is any expression cassette described herein. In some embodiments, the expression cassette is introduced into the plant cell using a recombinant DNA construct. In some embodiments, the expression cassette is introduced into the plant cell using a recombinant virus. In some embodiments, the recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV). In some embodiments, the expression cassette is introduced into the plant cell using a bacterium. In some embodiments, the bacterium is Agrobacterium tumefaciens. In some embodiments, the expression cassette is introduced into the plant cell by Agrobacterium-mediated transformation, biolistic transformation, by microinjection, or by electroporation.

In some embodiments, the genetically-modified plant cell comprises a maintainer construct on a nuclear chromosome, the maintainer construct comprises: (a) a copy of the male-essential mitochondrial gene which encodes a wild-type polypeptide; (b) a non-male promoter operably linked to the copy of the male-essential mitochondrial gene; and (c) a nucleic acid sequence encoding a maintainer MTP which is attached to the wild-type polypeptide. In some embodiments, the copy of the male-essential mitochondrial gene in the maintainer construct is codon-optimized for expression in the nucleus and encodes a wild-type polypeptide. In some embodiments, the copy of the male-essential mitochondrial gene in the maintainer construct encodes a wild-type polypeptide but is modified to not comprise the recognition sequence, the first recognition sequence, or the second recognition sequence. In some embodiments, the maintainer MTP is attached to the N-terminus of the wild-type polypeptide. In some embodiments, the maintainer MTP is attached to the C-terminus of the wild-type polypeptide. In some embodiments, the maintainer MTP is fused to the wild-type polypeptide. In some embodiments, the maintainer MTP is attached to the wild-type polypeptide by a polypeptide linker. In some embodiments, the maintainer MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the maintainer MTP comprises an amino acid sequence set forth in any one of SEQ ID NOs: 7-11. In some embodiments, the maintainer MTP comprises an amino acid sequence set forth in SEQ ID NO: 7. In some embodiments, the non-male promoter is a weak non-male promoter. In some embodiments, the non-male promoter is a CaMV35S promoter or an enhanced CaMV35S promoter. In some embodiments, the promoter is a strong non-male promoter. In some embodiments, the strong non-male promoter expresses said male-essential mitochondrial gene in comparable levels to the levels of the male-essential mitochondrial gene expressed from the mitochondrial gene.

In another aspect, the invention provides a genetically-modified plant cell made by any method described above.

In some embodiments, the genetically-modified plant cell is cultured into a genetically-modified plant comprising the modified male-essential plant mitochondrial gene. In some embodiments, the genetically-modified plant cell is a genetically-modified tobacco cell.

In another aspect, the invention provides a genetically-modified plant made by the preceding method above.

In some embodiments, the genetically-modified plant cell is cultured into a genetically-modified plant comprising the modified male-essential plant mitochondrial gene and the maintainer construct on a nuclear chromosome. In some embodiments, the genetically-modified plant cell is a genetically-modified tobacco cell.

In some embodiments, the genetically-modified plant cell is cultured into a genetically-modified plant comprising the modified male-essential plant mitochondrial gene and the maintainer construct on a nuclear chromosome, wherein the non-male promoter is a weak non-male promoter, and wherein the genetically-modified plant is unable to produce mature seed. In some embodiments the genetically-modified plant produces seedless fruit.

In another aspect, the invention provides a genetically-modified plant made by the preceding method above. In some embodiments, the genetically-modified plant comprises a weak non-male promoter and is unable to produce mature seed. In some embodiments, the genetically-modified plant produces seedless fruit.

In another aspect, the invention provides a method of producing hybrid seed, the method comprising: (a) crossing a genetically-modified plant described herein with a restorer plant comprising a restorer construct on a nuclear chromosome; and (b) culturing the crossed plant to produce hybrid seed; wherein the restorer construct comprises: (i) a copy of the male-essential mitochondrial gene which encodes a wild-type polypeptide; (ii) a ubiquitous promoter operably linked to the copy of the male-essential mitochondrial gene; and (iii) a nucleic acid sequence encoding a restorer MTP which is attached to the wild-type polypeptide; wherein the hybrid seed comprises the maintainer construct on a nuclear chromosome, the restorer construct on a nuclear chromosome, and the modified male-essential plant mitochondrial gene. In some embodiments, the copy of the male-essential mitochondrial gene in the restorer construct is codon-optimized for expression in the nucleus and encodes a wild-type polypeptide. In some embodiments, the copy of the male-essential mitochondrial gene in the restorer construct encodes a wild-type polypeptide but is modified to not comprise the recognition sequence, the first recognition sequence, or the second recognition sequence. In some embodiments, the restorer MTP is attached to the N-terminus of the wild-type polypeptide. In some embodiments, the restorer MTP is attached to the C-terminus of the wild-type polypeptide. In some embodiments, the restorer MTP is fused to the wild-type polypeptide. In some embodiments, the restorer MTP is attached to the wild-type polypeptide by a polypeptide linker. In some embodiments, the restorer MTP comprises an amino acid sequence having at least 80%, 85%, 90%, 91%, 92%, 93%, 94%, 95%, 96%, 97%, 98%, 99% or more sequence identity to a sequence set forth in any one of SEQ ID NOs: 7-11. In some embodiments, the restorer MTP comprises an amino acid sequence set forth in any one of SEQ ID NOs: 7-11. In some embodiments, the restorer MTP comprises an amino acid sequence set forth in SEQ ID NO: 7. In some embodiments, the ubiquitous active promoter is an mtATP promoter. In some embodiments, the ubiquitous promoter is a weak ubiquitous promoter. In some embodiments, the ubiquitous promoter is an mtATP promoter. In some embodiments, the ubiquitous promoter is a β-ATP promoter. In some embodiments, the ubiquitous promoter is a strong ubiquitous promoter. In some embodiments, the strong ubiquitous promoter is a ubiquitin promoter. In some embodiments the genetically modified plant described herein is an inbred plant and the restorer plant is an inbred plant. In some embodiments the genetically modified plant and the restorer plant used to produce hybrid seed are genetically diverse.

In one aspect, the invention provides a method of producing seedless fruit, the method comprising: (a) pollinating said genetically-modified plant disclosed herein with pollen from a wild-type plant or the maintainer plant disclosed herein; and (b) culturing the pollinated plant in order to obtain the seedless fruit.

In another aspect, the invention provides a hybrid seed produced by any method of producing a hybrid seed described herein.

In another aspect, the invention provides a method of producing seed of a plant comprising a cytoplasmic male sterility trait, the method comprising: (a) crossing a genetically-modified plant described herein with a maintainer plant comprising the maintainer construct on a nuclear chromosome and the male-essential plant mitochondrial gene; and (b) culturing the crossed plant to produce seed; wherein the seed comprises the maintainer construct on a nuclear chromosome and the modified male-essential plant mitochondrial gene.

In some aspects, the present disclosure provides an organelle-targeting engineered nuclease (OTEN) capable of binding and cleaving a recognition sequence in an organelle genome of a eukaryotic cell, wherein the OTEN comprises an engineered nuclease attached to a mitochondrial transit peptide (MTP), wherein the MTP comprises an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 10. In some aspects, the organelle is a mitochondrion or a chloroplast. In some aspects, the MTP comprises an amino acid sequence set forth in SEQ ID NO: 10.

In some aspects, the engineered nuclease of the OTEN is an engineered meganuclease, a CRISPR system nuclease, a compact TALEN, or a megaTAL.

In some embodiments, the MTP is attached to the C-terminus of the engineered nuclease. In some embodiments, the MTP is attached to the N-terminus of the engineered nuclease. In some embodiments, the MTP is fused to the engineered nuclease. In some embodiments, the MTP is attached to the engineered nuclease by a polypeptide linker.

In some embodiments, the engineered nuclease is attached to a first MTP and a second MTP, at least one of which is an MTP described herein. In some embodiments the first MTP and the second MTP are identical. In some embodiments, the first MTP and the second MTP are not identical. In some embodiments, the first MTP and/or the second MTP is fused to the engineered nuclease. In some embodiments, the first MTP and/or the second MTP is attached to the engineered nuclease by a polypeptide linker.

In some embodiments, the engineered nuclease is a zinc finger nuclease or a TALEN. In some embodiments, the MTP is attached to the N-terminus of the engineered nuclease. In some embodiments, the MTP is fused to the engineered nuclease. In some embodiments, the MTP is attached to the engineered nuclease by a polypeptide linker.

In some embodiments, the engineered nuclease is an engineered meganuclease, which comprises a first subunit and a second subunit; the first subunit binds to a first recognition half-site of the recognition sequence and comprises a first hypervariable (HVR1) region; the second subunit binds to a second recognition half-site of the recognition sequence and comprises a second hypervariable (HVR2) region; and the first subunit and the second subunit each comprise an amino acid sequence having at least 80%, 85%, 90%, 91%, 92%, 93%, 94%, 95%, 96%, 97%, 98%, 99% or more sequence identity to a sequence set forth in SEQ ID NO: 18. In some embodiments, the first subunit and the second subunit each comprise an amino acid sequence having at least 80%, 85%, 90%, 91%, 92%, 93%, 94%, 95%, 96%, 97%, 98%, 99% or more sequence identity to residues 7-153 of SEQ ID NO: 18. In some embodiments, the engineered meganuclease comprises an amino acid sequence having at least 80%, 85%, 90%, 91%, 92%, 93%, 94%, 95%, 96%, 97%, 98%, 99% or more sequence identity to a sequence set forth in SEQ ID NO: 31.

In some aspects, the present disclosure provides a polynucleotide comprising a nucleic acid sequence encoding an OTEN described herein. In some embodiments, the polynucleotide is an mRNA. In some embodiments, the polynucleotide further comprises a selectable marker. In some embodiments, the selectable marker is an antibiotic resistance gene.

In some aspects, the present disclosure provides a recombinant DNA construct comprising a polynucleotide comprising a nucleic acid sequence encoding an OTEN described herein. In some embodiments, the recombinant DNA construct encodes a recombinant virus comprising the polynucleotide. In some embodiments, the recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV). In some embodiments, the recombinant virus is a recombinant AAV.

In some embodiments, the polynucleotide of the recombinant DNA construct comprises a promoter operably linked to the nucleic acid sequence encoding the OTEN. In some embodiments, the promoter is a constitutive promoter, ubiquitous, or a tissue-specific promoter.

In some embodiments, the recombinant DNA construct comprises tDNA sequences.

In some aspects, the present disclosure provides a bacterium comprising the recombinant DNA construct described herein. In some embodiments, the bacterium is Agrobacterium tumefaciens.

In some aspects, the present disclosure provides a recombinant virus comprising a polynucleotide comprising a nucleic acid sequence encoding an OTEN described herein. In some embodiments, the recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV). In some embodiments, the recombinant virus is a recombinant AAV. In some embodiments, the recombinant AAV has an AAV1, AAV2, AAV3, AAV3B, AAV4, AAV5, AAV6, AAV7, AAV8, AAV9, AAV10, AAV11, or AAVHSC capsid.

In some embodiments, the polynucleotide of the recombinant virus comprises a promoter operably linked to the nucleic acid sequence encoding the engineered meganuclease. In some embodiments, the promoter is a constitutive promoter, ubiquitous promoter, or a tissue-specific promoter.

In some aspects, the present disclosure provides an expression cassette comprising a polynucleotide described herein. In some embodiments, the polynucleotide comprises a promoter operably linked to the nucleic acid sequence encoding the OTEN. In some embodiments, the promoter is a constitutive promoter, ubiquitous promoter, or a tissue-specific promoter.

In some aspects, the present disclosure provides a lipid nanoparticle composition comprising lipid nanoparticles comprising a polynucleotide, which comprises a nucleic acid sequence encoding an OTEN described herein. In some embodiments, the polynucleotide is an mRNA. In some embodiments, the polynucleotide is mRNA described herein.

In some aspects, the present disclosure provides a plant comprising a polynucleotide, a recombinant DNA construct, or an expression cassette comprising a nucleic acid sequence encoding an OTEN described herein.

In some aspects, the present disclosure provides an organelle-targeting recombinant AAV (OTAAV) comprising an MTP attached to a recombinant AAV, wherein the MTP comprises an amino acid sequence having at least 80%, 85%, 90%, 91%, 92%, 93%, 94%, 95%, 96%, 97%, 98%, 99% or more sequence identity to a sequence set forth in SEQ ID NO: 10. In some embodiments, the MTP comprises an amino acid sequence set forth in SEQ ID NO: 10. In some embodiments, the MTP is attached to a capsid protein of the recombinant AAV. In some embodiments, the MTP is fused to the recombinant AAV. In some embodiments, the MTP is attached to the recombinant AAV by a polypeptide linker.

In some embodiments, the OTAAV comprises a polynucleotide comprising a nucleic acid sequence encoding an engineered nuclease. In some embodiments, the engineered nuclease is an engineered meganuclease, a zinc finger nuclease, a TALEN, a CRISPR system nuclease, or a megaTAL. In some embodiments, the engineered nuclease is an OTEN described herein. In some embodiments, the polynucleotide comprises an expression construct described herein.

In some aspects, the present disclosure provides a genetically-modified eukaryotic cell comprising a polynucleotide described herein. In some aspects, the present disclosure provides a genetically-modified eukaryotic cell comprising an OTAAV described herein.

In some embodiments, the genetically-modified eukaryotic cell is a genetically-modified mammalian cell. In some embodiments, genetically-modified eukaryotic cell is a genetically-modified human cell. In some embodiments, the genetically-modified eukaryotic cell is a genetically-modified plant cell.

In some embodiments, the plant is tobacco, rice, maize, soybean, sugarcane, sorghum, millet, switchgrass, alfalfa, silage corn, hay, or Miscanthus sp.

In some aspects, a plant part or plant cell of the plant comprises a polynucleotide, a recombinant DNA construct, and/or an expression cassette described herein.

In some aspects, a seed produced from the plant comprises a polynucleotide, a recombinant DNA construct, and/or an expression cassette described herein.

In some aspects, the present disclosure provides a pharmaceutical composition comprising a pharmaceutically acceptable carrier and an OTEN described herein.

In some aspects, the present disclosure provides a pharmaceutical composition comprising a pharmaceutically acceptable carrier and a polynucleotide described herein.

In some aspects, the present disclosure provides a pharmaceutical composition comprising a pharmaceutically acceptable carrier and a recombinant DNA construct described herein.

In some aspects, the present disclosure provides a pharmaceutical composition comprising a pharmaceutically acceptable carrier and a recombinant virus described herein.

In some aspects, the present disclosure provides a pharmaceutical composition comprising a pharmaceutically acceptable carrier and a lipid nanoparticle composition described herein.

In some aspects, the present disclosure provides a pharmaceutical composition comprising a pharmaceutically acceptable carrier and an OTAAV described herein.

The foregoing and other aspects and embodiments of the present disclosure can be more fully understood by reference to the following detailed description and claims. Certain features of the disclosure, which are, for clarity, described in the context of separate embodiments, may also be provided in combination in a single embodiment. All combinations of the embodiments are specifically embraced by the present disclosure and are disclosed herein just as if each and every combination was individually and explicitly disclosed. Conversely, various features of the disclosure, which are, for brevity, described in the context of a single embodiment, may also be provided separately or in any suitable sub-combination. All sub-combinations of features listed in the embodiments are also specifically embraced by the present disclosure and are disclosed herein just as if each and every such sub-combination was individually and explicitly disclosed herein. Embodiments of each aspect of the present disclosure disclosed herein apply to each other aspect of the disclosure mutatis mutandis.

### BRIEF DESCRIPTION OF THE FIGURES

FIG. 1 represents a schematic of dual targeting of the mtATP1 mitochondrial gene by ATP 5-6 and ATP 7-8 nucleases with matching cut sites followed by transient annealing/Microhomology-mediated end joining (MMEJ) which results in a 674 bp deletion and truncated mtATP1 protein.

FIG. 2 shows the Chinese hamster ovary (CHO) iGFFP Bulk data for ATP 5-6 and ATP 7-8 variants. Selected variants (ATP 5-6x.87 and ATP 7-8x.9) are shaded black and the CHO 23-24 positive control is shaded white.

FIG. 3 is an example alignment of reads from MiniSeq sequencing of protoplasts transfected with mitochondrially targeted ATP 5-6 and ATP 7-8 nucleases. Reads contain a 674 bp deletion spanning the region between ATP 5-6 and ATP 7-8 cut sites.

FIG. 4 shows the number of aligned reads containing the deletion product from dual targeting of ATP 5-6 and ATP 7-8 nucleases in protoplasts of two tobacco varieties (TN90 and K326). No aligned reads with the deletion product were identified in protoplasts transfected with the single ATP 5-6 nuclease or GFP control.

FIG. 5 contains 20× (left) and 40× (right) microscope images of GFP fluorescence in tobacco protoplasts 24 h post-transfection with plasmid vectors comprised of fusions of transit peptides with MTEM and GFP coding sequences. Control (no transit peptide) images are in the top row and nuclear localization signal control images are in the bottom row.

FIG. 6 depicts the dual nuclease binary vector and control binary vectors. FIG. 6A is the ATP 5-6 and ATP 7-8 vector. FIG. 6B is the ATP 5-6 control binary vector. FIG. 6C is the ATP 7-8 control binary vector.

FIG. 7 depicts a model showing how maintainer lines and male-sterile lines produced as described herein could be used to propagate the male-sterile inbred line used for hybrid seed production. The plant on the top right is the CMS line that was developed by MTEM-mediated knockout of the mtATP1 gene (atp1-mut cytoplasm) within a hypothetical inbred (Inbred A) that possesses the repurposed nuclear version of that gene that is driven by a promoter that is expressed in all tissues of the plant except anthers (C-Aprom:nATP1). The plant on the top right is maintainer line that also possesses the C-Aprom:nATP1 construct in a homozygous state in the same Inbred A background, but has a normal cytoplasm and is thus male fertile. Crossing the maintainer line as the male parent to the CMS line as the female parent serves to propagate the CMS line.

FIG. 8 depicts a model showing how restorer lines and CMS lines produced as described herein could be used to produce hybrid seed. The plant on the top left is the restorer which contains a repurposed nATP1 gene under the control of a strong constitutive promoter βprom:nATP1) within a distinct inbred background (Inbred B). The restorer line as the male parent is crossed to the CMS line as female parent to produce hybrid seed. Although the hybrid seed possesses the mutant cytoplasm (atp1-mut), the hybrid plants are fertile because the Cprom:nATP1 restorer construct compensates for the atp-mut mutation during anther development.

FIG. 9 presents a semi-quantitative PCR analysis of tobacco K326 haploid plants transformed with βprom:nATP1 (FIG. 9A) and 35S:nATP1 (FIG. 9B). A tobacco actin gene was used as a control.

FIG. 10 presents a PCR analysis of 35S:nATP1 and βprom:nATP1 lines transformed with MTEM constructs targeting mtATP1 using primers specific to mtATP1 and mtATP6. WT=wild type; EV=empty vector control. Size marker ladder is shown in the first lane.

FIG. 11 reports a summary of the results of transformation of 35S:nATP1 and βprom:nATP1 plants that have been transformed with MTEM constructs.

FIG. 12 shows representative 35S:nATP1/ΔmtATP1 plants approximately six weeks after transplanting to soil.

FIG. 13 shows representative 35S:nATP1/ΔmtATP1 plants approximately ten weeks after transplanting to soil.

FIG. 14 shows representative βprom:nATP1/ΔmtATP1 plants approximately 12 weeks after transplanting to soil.

FIG. 15 shows flowers on 35S:nATP1/ΔmtATP1 and control plants

FIG. 16 shows pistils and stigmas on 35S:nATP1/ΔmtATP1 and control plants

FIG. 17 shows anthers on 35S:nATP1/ΔmtATP1 and control plants, with pollen production only observed on the controls.

FIG. 18 demonstrates that mature pods from 35S:nATP1 X ΔmtATP1 CMS crosses appear normal, in contrast to pods from unfertilized flowers which remain undeveloped.

FIG. 19 depicts 35S:nATP1 X 35S:nATP1/ΔmtATP1 CMS seeds that are smaller and lighter in color than those from self-fertilized empty vector controls.

FIG. 20 shows 35S:nATP1 X 35S:nATP1/ΔmtATP1 CMS seeds compared to normal seeds produced from a self-pollinated 35S:nATP1 plant. Tobacco seeds from plants that had normal cytoplasms mostly sank when placed in water, in contrast to the seed-like structures produced in 35S:nATP1 X 35S:nATP1/ΔmtATP1 pods that mostly floated to the top.

FIG. 21 shows that the average 100 seed weight observed from plants with normal cytoplasms (K326 WT and 35S:nATP1 selfed) was 8-11 times heavier than that observed from with crosses onto plants with mutant (ΔmtATP1) cytoplasms.

### BRIEF DESCRIPTION OF THE SEQUENCES

SEQ ID NO: 1 sets forth the nucleic acid sequence of the ATP 5-6 recognition sequence (sense).

SEQ ID NO: 2 sets forth the nucleic acid sequence of the ATP 7-8 recognition sequence (sense).

SEQ ID NO: 3 sets forth the amino acid sequence of the ATP 5-6x.87 meganuclease.

SEQ ID NO: 4 sets forth the nucleic acid sequence of the ATP 5-6x.87 meganuclease.

SEQ ID NO: 5 sets forth the amino acid sequence of the ATP 7-8x.9 meganuclease.

SEQ ID NO: 6 sets forth the nucleic acid sequence of the ATP 7-8x.9 meganuclease.

SEQ ID NO: 7 sets forth the amino acid sequence of an ATPase-β MTP.

SEQ ID NO: 8 sets forth the amino acid sequence of an ATPase-β MTP (minus 12 C-terminal amino acids).

SEQ ID NO: 9 sets forth the amino acid sequence of a COXIV MTP.

SEQ ID NO: 10 sets forth the amino acid sequence of an M20 MTP.

SEQ ID NO: 11 sets forth the amino acid sequence of a COXVIII-SU9 MTP.

SEQ ID NO: 12 sets forth the amino acid sequence of an MVMp NS2 NES.

SEQ ID NO: 13 sets forth the amino acid sequence of an NES.

SEQ ID NO: 14 sets forth the nucleic acid sequence of the ATP 5-6 and ATP 7-8 recognition sequence following cleavage and ligation of their complementary overhangs.

SEQ ID NO: 15 sets forth the nucleic acid sequence of the mtATP1 gene following cleavage and ligation of the ATP 5-6 and ATP 7-8 recognition sequences.

SEQ ID NO: 16 sets forth the nucleic acid sequence of the ATP 5-6 recognition sequence (antisense).

SEQ ID NO: 17 sets forth the nucleic acid sequence of the ATP 7-8 recognition sequence (antisense).

SEQ ID NO: 18 sets forth the amino acid sequence of a wild-type I-CreI meganuclease.

SEQ ID NO: 19 sets forth the cDNA sequence of transcripts derived from the tobacco mtATP1 gene. The t nucleotides at positions 1039, 1178, 1216, 1292, 1415, and 1490 represent the locations where RNA editing has converted Cs to Us in the final transcript (in the corresponding mtATP1 DNA sequence there are C nucleotides at these positions).

SEQ ID NO: 20 sets forth the sequence of nATP1. Positions 10-206 correspond to the region of the tobacco ATP2 gene that encodes the 54 aa transit peptide plus the first 12 aa of the β-subunit of the ATPase complex. Positions 207-1737 represent the mtATP1 reading frame of SEQ ID NO: 19 that had been codon optimized for expression in the nucleus. Positions 1744-2632 correspond to the 889 bp region directly 3′ of stop codon of the tobacco ATP2 gene. Positions 1744-2036 correspond to the ATP2 3′-UTR.

SEQ ID NO: 21 sets forth the sequence of the construct 35S:nATP1. Positions 7-853 correspond to an enhanced 35S CaMV promoter.

SEQ ID NO: 22 sets forth the sequence of the construct βprom:nATP1. Positions 7-2203 corresponds to a region predicted to include the promoter of the tobacco ATP2 gene. Positions 2103-2203 are included in the 5′-UTR.

SEQ ID NO: 23 sets forth the sequence of the nATP1-specific forward primer.

SEQ ID NO: 24 sets forth the sequence of the nATP1-specific reverse primer.

SEQ ID NO: 25 sets forth the sequence of the tobacco actin gene control forward primer.

SEQ ID NO: 26 sets forth the sequence of the tobacco actin gene control reverse primer.

SEQ ID NO: 27 sets forth the sequence of the wild-type mtATP1 gene forward primer flanking the ATP 5-6 and ATP 7-8 recognition sites.

SEQ ID NO: 28 sets forth the sequence of the wild-type mtATP1 gene reverse primer flaking the ATP 5-6 and ATP 7-8 recognition sites.

SEQ ID NO: 29 sets forth the sequence of the wild-type mitochondrial gene mtATP6 control forward primer.

SEQ ID NO: 30 sets forth the sequence of the wild-type mitochondrial gene mtATP6 control reverse primer.

SEQ ID NO: 31 sets forth the amino acid sequence of an ARCUS nuclease comprising wild-type I-CreI subunits.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, the term “5′ cap” (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, the term “allele” refers to one of two or more variant forms of a gene.

As used herein, the term “constitutive promoter” refers to a nucleotide sequence which, when operably linked with a polynucleotide which encodes or specifies a gene product, causes the gene product to be produced in a cell under most or all physiological conditions of the cell or at most or all times of cell development.

As used herein, the term “a control” or “a control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the term “corresponding to” with respect to modifications of two proteins or amino acid sequences is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment and despite the fact that X and Y may be different numbers.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “endogenous” in reference to a nucleotide sequence or protein is intended to mean a sequence or protein that is naturally comprised within or expressed by a cell.

As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention.

As used herein, the term “expression” refers to the transcription and/or translation of a particular nucleotide sequence driven by a promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, adeno-associated viruses, cucumber mosaic virus (CMV), tobacco mosaic virus (TMV), cauliflower mosaic virus (CaMV), odontoglossum ringspot virus (ORSV), tomato mosaic virus (ToMV), bamboo mosaic virus (BaMV), cowpea mosaic virus (CPMV), potato virus X (PVX), Bean yellow dwarf virus (BeYDV), turnip vein-clearing virus (TVCV)) that incorporate the recombinant polynucleotide.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.” For example, as used herein, a “genetically-modified” cell may refer to a cell wherein the mitochondrial DNA has been deliberately modified by recombinant technology.

As used herein, the term “homologous recombination” or “HR” refers to the natural, cellular process in which a double-stranded DNA-break is repaired using a homologous DNA sequence as the repair template (see, e.g., Cahill et al. (2006), Front. Biosci. 11:1958-1976). The homologous DNA sequence may be an endogenous chromosomal sequence or an exogenous nucleic acid that was delivered to the cell.

As used herein, the term “homology arms” or “sequences homologous to sequences flanking a nuclease cleavage site” refer to sequences flanking the 5′ and 3′ ends of a nucleic acid molecule, which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the term “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

As used herein, the term “lipid nanoparticle” refers to a lipid composition having a typically spherical structure with an average diameter between 10 and 1000 nanometers. In some formulations, lipid nanoparticles can comprise at least one cationic lipid, at least one non-cationic lipid, and at least one conjugated lipid. Lipid nanoparticles known in the art that are suitable for encapsulating nucleic acids, such as mRNA, are contemplated for use in the invention.

As used herein, the term “modification” with respect to recombinant proteins means any insertion, deletion, or substitution of an amino acid residue in the recombinant sequence relative to a reference sequence (e.g., a wild-type or a native sequence).

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain one or more introns.

As used herein, the term “operably linked” is intended to mean a functional linkage between two or more elements. For example, an operable linkage between a nucleic acid sequence encoding a nuclease as disclosed herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, unless specifically indicated otherwise, the word “or” is used in the inclusive sense of “and/or” and not the exclusive sense of “either/or.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “reduced” or “decreased” refers to a reduction in the expression of a male-essential gene from mtDNA or the reduction in the presentation of a fertile male phenoptype in a plant when compared to a population of control cells or control plant. Such a reduction is up to 5%, 10%, 20%, 30%, 40%, 50%, 60%, 70%, 80%, 90%, 95%, or up to 100%. Accordingly, the term “reduced” encompasses both a partial knockdown and a complete knockdown of the expression of a male-essential gene or a male phenotype.

As used herein, the term “male-essential gene” or “male-essential plant mitochondrial gene” refers to a gene encoding a polypeptide or regulatory sequence necessary for expression of a fertile male phenotype having an ability to fertilize a flower of the same plant species, including a plant mitochondrial ATPase (mtATPase), such as the mtATPase1 or mtATP1 gene (UniProt accession number Q9XPJ9 (ATPA_DICDI)). Other male-essential genes include but are not limited to: ATP4, ATP6, ATP8, ATP9, COXI, COXII, COXIII, COB, NAD1, NAD5, and active variants and fragments thereof. The term male-essential gene also refers to naturally occurring DNA sequence variations of any male-essential gene listed herein. As used herein, male-essential mitochondrial genes need not be specific for the male phenotype, but could be any mitochondrial genes essential for normal plant development, including the development of a fertile male phenotype. Accordingly, the male-essential mitochondrial gene can also be referred to as an essential or plant essential mitochondrial gene.

As used herein, the term “male sterile” refers to the absence or substantial absence of male fertility. For example, male sterility can result from deficiencies in the formation of anther, pollen, sporogenous tissue, and/or any other cells or tissue necessary for male fertility in a plant.

As used herein, a “nuclear export sequence” or “nuclear export signal” or “NES” refers to a peptide that is attached to a protein in order to facilitate the export of the protein from the cell nucleus to the cytoplasm.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein, the term “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, the term “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, the term “promoter” or “regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, the terms “transfected” or “transformed” or “transduced” or “nucleofected” refer to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

As used herein, the term “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviral vectors, lentiviral vectors, adenoviral vectors, and adeno-associated viral vectors (AAV).

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “altered specificity,” when referencing to a nuclease, means that a nuclease binds to and cleaves a recognition sequence, which is not bound to and cleaved by a reference nuclease (e.g., a wild-type) under physiological conditions, or that the rate of cleavage of a recognition sequence is increased or decreased by a biologically significant amount (e.g., at least 2×, or 2×-10×) relative to a reference nuclease.

As used herein, the term “center sequence” refers to the four base pairs separating half-sites in the meganuclease recognition sequence. These bases are numbered+1 through +4. The center sequence comprises the four bases that become the 3′ single-strand overhangs following meganuclease cleavage. “Center sequence” can refer to the sequence of the sense strand or the antisense (opposite) strand. Meganucleases are symmetric and recognize bases equally on both the sense and antisense strand of the center sequence. For example, the sequence A+1A+2A+3A+4 on the sense strand is recognized by a meganuclease as T+1T+2T+3T+4 on the antisense strand and, thus, A+1A+2A+3A+4 and T+1T+2T+3T+4 are functionally equivalent (e.g., both can be cleaved by a given meganuclease). Thus, the sequence C+1T+2G+3C+4, is equivalent to its opposite strand sequence, G+1C+2A+3G+4 due to the fact that the meganuclease binds its recognition sequence as a symmetric homodimer.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of SEQ ID NO: 3 or 5. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of SEQ ID NO: 3 or 5. In other embodiments, variable residues within a hypervariable region correspond to one or more of positions 215, 217, 219, 221, 223, 224, 229, 231, 233, 235, 237, 259, 261, 266, and 268 of SEQ ID NO: 3 or 5.

As used herein, the term “linker” refers to an exogenous peptide sequence used to join two nuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety. In some embodiments, a linker may have an amino acid sequence comprising residues 154-195 of SEQ ID NO: 3 or 5.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 18), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the terms “nuclease” and “endonuclease” are used interchangeably to refer to naturally-occurring or engineered enzymes, which cleave a phosphodiester bond within a polynucleotide chain.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the terms “target site” or “target sequence” refers to a region of the chromosomal DNA of a cell comprising a recognition sequence for a nuclease.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, End1, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as CpfI. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific homing endonuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, 51 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, 51 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “mitochondria-targeting engineered nuclease” or “MTEN” refers to a nuclease, such as an engineered nuclease, attached to a peptide or other molecule that is capable of directing the nuclease to the mitochondria such that the nuclease is capable of cleaving mitochondrial DNA within the mitochondrial organelle. The engineered nuclease portion of the MTEN can be an engineered meganuclease, a CRISPR system nuclease, a compact TALEN, or a megaTAL. As used herein, the term MTEN includes MTEM defined elsewhere herein.

As used herein, the term “mitochondria-targeting engineered meganuclease” or “MTEM” refers to an engineered meganuclease attached to a peptide or other molecule that is capable of directing the engineered meganuclease to the mitochondria such that the engineered meganuclease is capable of binding and cleaving mitochondrial DNA within the mitochondrial organelle. As used herein, the term MTEM is an example of an MTEN defined elsewhere herein.

As used herein the term “mitochondrial transit peptide” or “MTP” refers to a peptide or fragment of amino acids that can be attached to a separate molecule in order to transport the molecule in the mitochondria. For example, an MTP can be attached to a nuclease, such as an engineered meganuclease, in order to transport the engineered meganuclease into the mitochondria. MTPs can consist of an alternating pattern of hydrophobic and positively charged amino acids to form what is called amphipathic helix.

As used herein, a “vector” can also refer to a viral vector. Viral vectors (i.e., a recombinant virus) can include, without limitation, retroviral vectors, lentiviral vectors, adenoviral vectors, and adeno-associated viral vectors (AAV).

As used herein, the term “serotype” or “capsid” refers to a distinct variant within a species of virus that is determined based on the viral cell surface antigens. Known serotypes of AAV, for example, include AAV1, AAV2, AAV3, AAV3B, AAV4, AAV5, AAV6, AAV7, AAV8, AAV9, AAV10, and AAV11 (Weitzman and Linden (2011) In Snyder and Moullier Adeno-associated virus methods and protocols. Totowa, NJ: Humana Press).

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions, stimuli, or further genetic modifications that would induce expression of altered genotype or phenotype.

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values and if the variable is inherently continuous.

### 2.1 Principle of the Invention

Mitochondria regulate cellular energy and metabolism under normal growth and development, as well as in response to stress. Thus, editing of the mitochondrial genome has diverse applications in both animals and plants. In humans, deleterious mitochondrial mutations are the source of a number of disorders for which gene editing therapies could be applied. However, albeit the potentials of using mitochondrial genome editing for therapeutic applications, it still remains an underexplored area of science because of the inability to efficiently target mitochondrial DNA (mtDNA) and generate precise edits. The mitochondrial genome is difficult to edit as the editing technology needs to be delivered to this organelle. There is particular difficulty in editing essential mitochondrial genes because eliminating their function would produce a lethal phenotype. All previous attempts at editing plant mitochondrial genome have thus targeted nonessential or redundant genes. Hence, there is a need for compositions and methods that would allow targeting and editing of essential mitochondrial genes in a manner that can maintain plant viability.

Moreover, in plants, extra-nuclear genetic information is found in both chloroplasts and mitochondria. Despite the fact that the total number of genes encoded by the chloroplast and mitochondrial genomes is minimal in comparison to that found in the nuclear genome, organelle-encoded genes play a critical role in core biological functions such as photosynthesis and cellular respiration. Traits of specific agronomic interest have also been shown to be encoded by plant organellar genomes, including herbicide resistance (chloroplast genome) and cytoplasmic male sterility (CMS; mitochondrial genome).

The present disclosure provides compositions and methods for binding and cleaving a recognition sequence of an essential mitochondrial gene by first transferring tis function to the nucleus. Disclosed herein are engineered nucleases (e.g., engineered meganucleases) attached to MTPs such that DSBs can be generated in the mtDNA. The present invention demonstrates that engineered nucleases (e.g., engineered meganucleases) can be directed into the mitochondria organelle and facilitate editing of essential mitochondrial genes, thus opening up an entire field of prospects and opportunities in life sciences. The compositions and methods disclosed herein establish the mechanisms for modifying plant mitochondrial genomes in order to establish a novel CMS system that could be utilized for efficient hybrid seed production in crops. The use of weak promoters in the disclosed CMS system is surprisingly shown to generate plants that are incapable of producing mature seed, and thus the CMS system could be utilized to generate seedless fruits.

### 2.2 Mitochondria-Targeting Engineered Nucleases for Recognizing and Cleaving Recognition Sequences within the Plant Mitochondrial DNA

Mitochondria-targeting engineered nucleases (MTENs), constructed of an engineered nuclease attached to a mitochondrial transit peptide (MTP), can effectively traffic from the cytoplasm of a eukaryotic cell into the mitochondria. Particular examples of MTENs include mitochondria-targeting engineered meganucleases (MTEMs). Once inside the mitochondrial organelle, the MTEN/MTEM can bind and cleave a recognition sequence in the mitochondrial genome. It is known in the art that it is possible to use a site-specific nuclease to make a DNA break in the nuclear genome of a living cell, and that such a DNA break can result in permanent modification of the genome. In nuclear genomes NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele on a nuclear genome. Further, the use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus. Such exogenous nucleic acids can encode any sequence or polypeptide of interest. Homologous recombination-mediated repair can be used to introduce mutations at cleaved recognition sequences of the mitochondrial genome. Moreover, repair of the mitochondrial genome resulting in deletions can be facilitated by regions of micro homology.

In some embodiments, the nucleases used to practice the invention are meganucleases, which generate MTEMs. In particular embodiments, the nucleases used to practice the invention are single-chain meganucleases. A single-chain meganuclease comprises an N-terminal subunit and a C-terminal subunit joined by a linker peptide. Each of the two domains recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

In some embodiments, an engineered meganuclease of the invention has been engineered to bind and cleave an ATP 5-6 recognition sequence (SEQ ID NO: 1) within a mitochondrial ATP synthase complex (mtATP) gene. Such engineered meganuclease is referred to herein as “ATP 5-6 meganuclease” or “ATP 5-6 nucleases”. An exemplary ATP 5-6 meganuclease is provided in SEQ ID NO: 3. In specific embodiments, the ATP-5-6 meganuclease is attached to an MTP to form an MTEM that cleaves the ATP 5-6 recognition sequence of SEQ ID NO: 1.

In other embodiments, an engineered meganuclease of the invention has been engineered to bind and cleave an ATP 7-8 recognition sequence (SEQ ID NO: 2) within a mitochondrial ATP synthase complex (mtATP) gene. Such engineered meganuclease is referred to herein as “ATP 7-8 meganuclease” or “ATP 7-8 nucleases”. An exemplary ATP 7-8 meganuclease is provided in SEQ ID NO: 5. In some embodiments, the ATP 7-8 meganuclease is attached to an MTP to for an MTEM that cleaves the ATP 7-8 recognition sequence of SEQ ID NO: 2.

Engineered meganucleases of the invention can comprise a first subunit, comprising a first hypervariable (HVR1) region, and a second subunit, comprising a second hypervariable (HVR2) region. Further, the first subunit can bind to a first recognition half-site in the recognition sequence (e.g., the ATP 5 half-site), and the second subunit can bind to a second recognition half-site in the recognition sequence (e.g., the ATP 6 half-site). In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit.

In some embodiments of the invention, the engineered meganuclease is an ATP 5-6 nuclease. In some such embodiments, the engineered meganuclease binds and cleaves a recognition sequence comprising SEQ ID NO: 1 within mitochondrial genome, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region. In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 3. In some such embodiments, the HVR1 region comprises one or more residues corresponding to residues 24, 26, 28, 30, 32, 33, 38, 40, 42, 44, 46, 68, 70, 75, and 77 of SEQ ID NO: 3. In some such embodiments, the HVR1 region comprises residues corresponding to residues 24, 26, 28, 30, 32, 33, 38, 40, 42, 44, 46, 68, 70, 75, and 77 of SEQ ID NO: 3. In some such embodiments, the HVR1 region comprises Y, R, K, or D at a residue corresponding to residue 66 of SEQ ID NO: 3. In some such embodiments, the HVR1 region comprises residues 24-79 of SEQ ID NO: 3. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 3. In some such embodiments, the HVR2 region comprises one or more residues corresponding to residues 215, 217, 219, 221, 223, 224, 229, 231, 233, 235, 237, 259, 261, 266, and 268 of SEQ ID NO: 3. In some such embodiments, the HVR2 region comprises residues corresponding to residues 215, 217, 219, 221, 223, 224, 229, 231, 233, 235, 237, 259, 261, 266, and 268 of any one of SEQ ID NO: 3. In some such embodiments, the HVR2 region comprises Y, R, K, or D at a residue corresponding to residue 257 of any one of SEQ ID NO: 3. In some such embodiments, the HVR2 region comprises residues 215-270 of SEQ ID NO: 3. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 3, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 3. In some such embodiments, the first subunit comprises G, S, or A at a residue corresponding to residue 19 of SEQ ID NO: 3. In some such embodiments, the first subunit comprises E, Q, or K at a residue corresponding to residue 80 of SEQ ID NO: 3. In some such embodiments, the second subunit comprises G, S, or A at a residue corresponding to residue 210 of SEQ ID NO: 3. In some such embodiments, the second subunit comprises E, Q, or K at a residue corresponding to residue 271 of SEQ ID NO: 3. In some such embodiments, the first subunit comprises a residue corresponding to residue 80 of SEQ ID NO: 3. In some such embodiments, the second subunit comprises a residue corresponding to residue 271 of SEQ ID NO: 3. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 3. In some such embodiments, the engineered meganuclease comprises the amino acid sequence of SEQ ID NO: 3. In some embodiments, the engineered meganuclease is encoded by a nucleic sequence having at least 80%, at least 85%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 4. In some such embodiments, the engineered meganuclease is encoded by the nucleic acid sequence set forth in SEQ ID NO: 4.

In some embodiments of the invention, the engineered meganuclease is an ATP 7-8 nuclease. In some such embodiments, the engineered meganuclease binds and cleaves a recognition sequence comprising SEQ ID NO: 2 within mitochondrial genome, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region. In some embodiments, the HVR1 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 24-79 of SEQ ID NO: 5. In some such embodiments, the HVR1 region comprises one or more residues corresponding to residues 24, 26, 28, 30, 32, 33, 38, 40, 42, 44, 46, 68, 70, 75, and 77 of SEQ ID NO: 5. In some such embodiments, the HVR1 region comprises residues corresponding to residues 24, 26, 28, 30, 32, 33, 38, 40, 42, 44, 46, 68, 70, 75, and 77 of SEQ ID NO: 5. In some such embodiments, the HVR1 region comprises Y, R, K, or D at a residue corresponding to residue 66 of SEQ ID NO: 5. In some such embodiments, the HVR1 region comprises residues 24-79 of SEQ ID NO: 5. In some such embodiments, the HVR2 region comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence corresponding to residues 215-270 of SEQ ID NO: 5. In some such embodiments, the HVR2 region comprises one or more residues corresponding to residues 215, 217, 219, 221, 223, 224, 229, 231, 233, 235, 237, 259, 261, 266, and 268 of SEQ ID NO: 5. In some such embodiments, the HVR2 region comprises residues corresponding to residues 215, 217, 219, 221, 223, 224, 229, 231, 233, 235, 237, 259, 261, 266, and 268 of any one of SEQ ID NO: 5. In some such embodiments, the HVR2 region comprises Y, R, K, or D at a residue corresponding to residue 257 of any one of SEQ ID NO: 5. In some such embodiments, the HVR2 region comprises residues 215-270 of SEQ ID NO: 5. In some such embodiments, the first subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 7-153 of SEQ ID NO: 5, and wherein the second subunit comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to residues 198-344 of SEQ ID NO: 5. In some such embodiments, the first subunit comprises G, S, or A at a residue corresponding to residue 19 of SEQ ID NO: 5. In some such embodiments, the first subunit comprises E, Q, or K at a residue corresponding to residue 80 of SEQ ID NO: 5. In some such embodiments, the second subunit comprises G, S, or A at a residue corresponding to residue 210 of SEQ ID NO: 5. In some such embodiments, the second subunit comprises E, Q, or K at a residue corresponding to residue 271 of SEQ ID NO: 5. In some such embodiments, the first subunit comprises a residue corresponding to residue 80 of SEQ ID NO: 5. In some such embodiments, the second subunit comprises a residue corresponding to residue 271 of SEQ ID NO: 5. In some such embodiments, the engineered meganuclease is a single-chain meganuclease comprising a linker, wherein the linker covalently joins the first subunit and the second subunit. In some such embodiments, the engineered meganuclease comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 5. In some such embodiments, the engineered meganuclease comprises the amino acid sequence of SEQ ID NO: 5. In some embodiments, the engineered meganuclease is encoded by a nucleic sequence having at least 80%, at least 85%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 6. In some such embodiments, the engineered meganuclease is encoded by the nucleic acid sequence set forth in SEQ ID NO: 6.

In addition to MTEMs, any engineered nuclease can be attached to an MTP to generate a mitochondria-targeted engineered nuclease (MTEN) including, for example, a zinc finger nuclease, a TALEN, a compact TALEN, a CRISPR system nuclease, or a megaTAL. Zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR system comprises two components: (1) a CRISPR nuclease; and (2) a short “guide RNA” comprising a ˜20 nucleotide targeting sequence that directs the nuclease to a location of interest in the genome. The CRISPR system may also comprise a tracrRNA. By expressing multiple guide RNAs in the same cell, each having a different targeting sequence, it is possible to target DNA breaks simultaneously to multiple sites in the genome.

In certain embodiments, the cleavage of a first and a second recognition sequence in the mitochondrial genome creates overhangs or “sticky ends” or complementary overhangs at the cleavage sites. Complementary overhangs can be designed to anneal in order to prevent degradation of the mitochondrial genome resulting from a DSB. The overhangs can be 3′ or 5′ overhangs and can create 1, 2, 3, 4, 5, 6, 7, 8, 9, or 10 base pair overhangs following cleavage. In particular embodiments, cleavage of engineered meganuclease recognition sequences, such as the ATP 5-6 recognition sequence and ATP 7-8 recognition sequence, creates 3′ overhangs that are 4 base pairs long. Sticky ends created by cleavage with engineered meganucleases, such as the ATP 5-6 and ATP 7-8 engineered meganucleases described herein, can be used to directly anneal the complementary overhangs. The complementary overhangs can be annealed transiently or with any enzyme-mediated method known in the art. In specific embodiments, the mtATP1 mitochondrial gene is cleaved by the ATP 5-6 and ATP 7-8 engineered meganucleases with matching cut sites followed by transient annealing/Microhomology-mediated end joining (MMEJ) resulting in a ˜674 bp deletion and truncated mtATP1 protein. In specific embodiments, the mitochondrial genome comprises SEQ ID NO: 14, which is formed following cleavage of the ATP 5-6 recognition sequence and ATP 7-8 recognition sequence and subsequent annealing/ligation of the overhangs created by cleavage of each recognition sequence. Accordingly, following cleavage of the ATP 5-6 recognition sequence and ATP 7-8 recognition sequence and subsequent annealing/ligation of the overhangs created by cleavage of each recognition sequence the sequence of the mtATP1 gene can have the nucleic acid sequence set forth in SEQ ID NO: 15. In some embodiments, SEQ ID NO: 15 represents an example of a truncated male-essential gene or and truncated mtATP1 gene. In some embodiments, the truncated mtATP1 protein has reduced activity and the plant or plant part is at least partially male sterile as a result of the deletion in the mtATP1 gene. For example, the activity of the mtATP1 protein can be reduced by about 5-10%, 10-20%, 20-30%, 30-40%, 40-50%, 50-60%, 60-70%, 70-80%, 80-90%, 90-100%, or more (e.g., by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, or more). In some embodiments, the truncated mtATP1 protein is inactive and the plant or plant part is male sterile as a result of the deletion in the mtATP1 gene.

The complementary overhangs can also be used to insert an exogenous sequence of interest into the gene between the nuclease cleavage sites. One advantage of using such a sticky end ligation method is that the insertion orientation of the exogenous sequence of interest can be carefully controlled. In some embodiments, an exogenous sequence of interest is flanked by different recognition sequences corresponding to different endogenous recognition sequences such that a region of the mitochondrial genome is deleted and replaced by the exogenous sequence of interest.

MTPs for directing nucleases into the mitochondria of plant cells can be from 10-100 amino acids in length. In specific embodiments, the MTP is about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, about 90, about 95, about 100, or more amino acids long. MTPs can contain additional signals that subsequently target the protein to different regions of the mitochondria, such as the mitochondrial matrix. Non limiting examples of MTPs for use in the compositions and methods disclose herein include, Neurospora crassa F0 ATPase subunit 9 (SU9) MTP, human cytochrome c oxidase subunit VIII (CoxVIII or Cox8) MTP, the P1 isoform of subunit c of human ATP synthase MTP, aldehyde dehydrogenase targeting sequence MTP, Glutaredoxin 5 MTP, Pyruvate dehydrogenase MTP, Peptidyl-prolyl isomerase MTP, Acetyltransferase MTP, Isocitrate dehydrogenase MTP, cytochrome oxidase MTP, and the subunits of the FA portion of ATP synthase MTP, CPN60/No GGlinker MTP, Superoxide dismutase (SOD) MTP, Superoxide dismutase doubled (2SOD) MTP, Superoxide dismutase modified (SODmod) MTP, Superoxide dismutase modified (2SODmod) doubled MTP, L29 MTP, gATPase gamma subunit (FAγ51) MTP, CoxIV twin strep (ABM97483) MTP, and CoxIV 10×His MTP.

In specific embodiments, the MTEN or MTEM comprises a combination of at least two MTPs. The combination of MTPs can be a combination of identical MTPs or a combination of different MTPs. In specific embodiments, the MTP comprises the Cox VIII MTP and the SU9 MTP into a single MTP represented by SEQ ID NO: 11. In some embodiments, the MTP is a COXIV MTP represented by SEQ ID NO: 9, the M20 MTP represented by SEQ ID NO: 10, the ATPase-β MTP represented by SEQ ID NO: 7, or ATPase-β MTP (minus 12 C-terminal amino acids) represented by SEQ ID NO: 8.

In order to form an MTEM or an MTEN, an MTP can be attached by any appropriate means to a nuclease disclosed herein. In specific embodiments, the MTP can be attached to the N-terminus of the nuclease. In other embodiments the MTP can be attached to the C-terminus of the nuclease. In some embodiments multiple MTPs can be attached to a single engineered meganuclease to form an MTEM, or can be attached to a CRISPR system nuclease, a TALEN, a compact TALEN, or a megaTAL to form an MTEN.

In some examples, a first MTP can be attached to the N-terminus of the nuclease and a second MTP can be attached to the C-terminus of the nuclease. In some embodiments, the first and second MTP are identical and in other embodiments, the first and second MTP not identical.

The MTP(s) can be attached by any means that allows for transport of the engineered nuclease into the mitochondria of a cell. In specific embodiments, the MTP is attached by fusing the MTP to the N- or C-terminus of the nuclease. The MTP can also be attached to the N-terminus or C-terminus of a nuclease by a peptide linker. The linker can be, for example, about 1, 2, 3, 4, 5, 6, 7, 8, 9, 10, 11, 12, 15, or 20 amino acids. In specific embodiments the MTP is attached to a peptide linker at the N- or C-terminus of the engineered meganuclease or other nuclease.

In some embodiments, an MTEM or MTEN for use in the compositions and methods of the present disclosure is attached to a nuclear export sequence (NES) in order to help prevent the engineered nuclease from cleaving the nuclear genome. In some such embodiments, the NES comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 13. For example, the NES may comprise the amino acid sequence of SEQ ID NO: 13. In certain embodiments, the NES is attached at the N-terminus of the MTEM or MTEN. In other embodiments, the NES is attached at the C-terminus of the MTEM or MTEN. In certain embodiments, the NES is fused to the MTEM or MTEN. In certain embodiments, the NES is attached to the MTEM or MTEN by a polypeptide linker.

In specific embodiments, the MTEM or MTEN is attached to multiple NESs. For example, an MTEM or MTEN disclosed herein can comprise a first NES and a second NES. In some such embodiments, the first NES is attached at the N-terminus of the MTEM or MTEN, and the second NES is attached at the C-terminus of the MTEM or MTEN. In some such embodiments, the first NES and/or the second NES comprises an amino acid sequence having at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 13. For example, the first NES and/or the second NES may comprise the amino acid sequence set forth in SEQ ID NO: 13. In some embodiments, the first NES and the second NES are identical. In other embodiments, the first NES and the second NES are not identical. The NES can be attached to the MTEM or MTEN by any appropriate means known in the art. For example, the first NES and/or the second NES can be fused to the MTEM or MTEN. In some embodiments, the first NES and/or the second NES is attached to the MTEM or MTEN by a polypeptide linker.

An MTEM or MTEN with an NES may have reduced or decreased transport to the nucleus of a target cell or target cell population (e.g., a eukaryotic cell or eukaryotic cell population), compared to an MTEM or MTEN without an NES. For example, nuclear transport of an MTEM or MTEN with an NES may be less than that of an MTEM or MTEN without an NES, by about 5-10%, 10-20%, 20-30%, 30-40%, 40-50%, 50-60%, 60-70%, 70-80%, 80-90%, 90-100%, or more (e.g., by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, or more). In some embodiments, an MTEM or MTEN with an NES may induce fewer nuclear indels (i.e., less cleavage and resulting deletion in nuclear genome of a target cell or target cell population) compared to an MTEM or MTEN without an NES. For example, nuclear indels induced by an MTEM or MTEN with an NES may be less than that induced by an MTEM or MTEN without an NES, by about 5-10%, 10-20%, 20-30%, 30-40%, 40-50%, 50-60%, 60-70%, 70-80%, 80-90%, 90-100%, or more.

### 2.3 Expression Cassettes

Also described herein are expression cassettes that can be used in a method for targeting and/or editing the mitochondrial genome, such as mitochondrial DNA (mtDNA). An expression cassette of the present disclosure may contain a polynucleotide that comprises a nucleic acid sequence encoding an MTEN or MTEM described herein. The polynucleotides provided herein can be mRNA or DNA. In particular embodiments, the polynucleotides further comprise a sequence encoding a selectable marker. The selectable marker can be any marker that allows selection of cells or organisms (e.g., bacteria, eukaryotic cells, mammalian cells, plant cells, plants, and/or plant parts) that contain a polynucleotide disclosed herein. In specific embodiments, the selectable marker is an antibiotic resistance gene.

In some embodiments, an expression cassette contains more than one polynucleotide. For example, an expression cassette of the present disclosure may contain two polynucleotides, such as a first polynucleotide containing a nucleic acid sequence encoding a first MTEN that binds and cleaves a first recognition sequence, and a second polynucleotide containing a nucleic acid sequence encoding a second MTEN that binds and cleaves a second recognition sequence. The first and second recognition sequences can be selected to have sequences that are capable of annealing following cleavage by the first and second MTEN, respectively. For example, the first recognition sequence and the second recognition sequence can have identical 4 basepair center sequences, such that cleavage of the recognition sequences produces complementary overhangs or sticky ends to facilitate transient annealing of the cleaved ends in order to produce a deletion of the region of the mitochondrial genome between the first and second recognitions sequences. In some embodiments, the first recognition sequence and second recognition sequence are in a male-essential plant mitochondrial gene. As described elsewhere herein, male-essential mitochondrial genes encode a protein or regulatory element that is essential for male fertility. In specific embodiments, the first recognition sequence and second recognition sequence are in an mtATPase gene, such mtATP1 gene that is essential for male fertility. In some embodiments, the first and second recognition sequences are less than about bout 1500, about 1400, about 1300, about 1200, about 1100, about 1000, about 900, about 800, about 700, about 600, about 500, about 400, about 300, about 200, about 100, or about 50 basepairs apart in the male-essential plant mitochondrial gene.

In particular, an expression cassette of the present disclosure may contain two polynucleotides, such as a first polynucleotide containing a nucleic acid sequence encoding an MTEM comprising an ATP 5-6 nuclease that has specificity for ATP 5-6 recognition sequence (SEQ ID NO: 1) within a mitochondrial ATP synthase complex (mtATP) gene; and a second polynucleotide containing a nucleic acid sequence encoding an MTEM comprising an ATP 7-8 nuclease that has specificity for ATP 7-8 recognition sequence (SEQ ID NO: 2) within a male-essential mitochondrial gene. In specific embodiments, the male-essential mitochondrial gene is the mtATP1 gene.

In some embodiments, an expression cassette disclosed herein may contain a promoter sequence for expression of the polynucleotides disclosed herein in a plant, plant part, or plant cell. In embodiments wherein two separate polynucleotides are located on an expression cassette, a single promoter can be operably linked to a polynucleotide having a nucleotide sequence encoding a first MTEN or MTEM and a second polynucleotide having a nucleotide sequence encoding a second MTEN or MTEM. In such embodiments wherein a single promoter is operably linked to two separate polynucleotides disclosed herein, the nucleic acid sequence encoding said first MTEN and said second nucleic acid sequence encoding said second MTEN are separated by an IRES or 2A sequence. In particular embodiments, the 2A sequence is T2A, a P2A, an E2A, or an F2A sequence.

In particular embodiments, the expression cassettes disclosed herein have a first promoter that is operably linked to a nucleic acid sequence encoding a first MTEN, and a second promoter that is operably linked to a nucleic acid sequence encoding a second MTEN. The promoters can be identical or not identical.

A number of promoters can be used in the various expression cassettes provided herein and each can be selected based on the desired outcome. It is recognized that different applications can be enhanced by the use of different promoters in the expression cassettes to modulate the timing, location and/or level of expression of the MTEN or MTEM of the present disclosure. Such expression cassettes may also contain, if desired, a promoter regulatory region (e.g., one conferring ubiquitous, inducible, constitutive, environmentally- or developmentally-regulated, or cell- or tissue-specific/selective expression), a transcription initiation start site, a ribosome binding site, an RNA processing signal, a transcription termination site, and/or a polyadenylation signal. A promoter can be said to be “ubiquitous” when it drives transcription in many, but not necessarily all, plant tissues. The ubiquitous promoters disclosed herein drive transcription in many plant tissues, at least including the male plant tissues identified herein. Examples of ubiquitous promoters that can be used as described herein include plant ubiquitin promoter (Ubi), rice actin 1 promoter (Act-I), maize alcohol dehydrogenase 1 promoter (Adh-1), mannopine synthase (MAS) promoter, 1′ or 2′ promoters derived from T-DNA of Agrobacterium tumefaciens, figwort mosaic virus 34S promoter, actin promoters such as the rice actin promoter, and ubiquitin promoters such as the maize ubiquitin-1 promoter. Yet other examples of ubiquitous promoters include p326, YP0144, YP0190, p13879, YP0050, p32449, 21876, YP0158, YP0214, YP0380, PT0848, and PT0633 promoters. A ubiquitous promoter may also be referred to as a constitutive promoter and can drive transcription of an operably linked nucleic acid molecule in most cell types at most times. In particular embodiments, the promoter can be constitutive and tissue-preferred such that the MTEM or MTEN is constitutively expressed in a tissue-preferred manner. Likewise, the promoter can be inducible and tissue-preferred. Examples of constitutive promoters include the rice actin promoter (McElroy et al. (1990) Plant Cell 2:163-171); ubiquitin promoter (Christensen et al. (1989) Plant Mol. Biol. 12:619-632; Christensen et al. (1992) Plant Mol. Biol. 18:675-689); pEMU (Last et al. (1991) Theor. Appl. Genet. 81:581-588); MAS promoter (Velten et al. (1984) EMBO J. 3:2723-2730), the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), and the like. Other constitutive promoters include, for example, those described in U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

In specific embodiments, the promoters disclosed herein are weak promoters. For example, a weak ubiquitous promoter and/or a weak non-male promoter can be operably linked to a male-essential mitochondrial gene (e.g., mtATP1). When introduced as part of a restorer or maintainer construct, a male-essential mitochondrial gene operably linked to a weak promoter is not expressed in a sufficient amount to restore wild-type activity of an inactivated mitochondrial copy of the male-essential gene. Thus, in some embodiments the use of weak promoter operably linked to a male-essential mitochondrial gene can produce a fruit deficient in mature seed and/or a seedless fruit.

The promoters disclosed herein can be strong promoters. For example, a strong ubiquitous promoter and/or a strong non-male promoter can be operably linked to a male-essential mitochondrial gene (e.g., mtATP1). When introduced as part of a restorer or maintainer construct, a male-essential mitochondrial gene operably linked to a weak promoter is expressed in a sufficient amount to restore wild-type activity of an inactivated mitochondrial copy of the male-essential gene. Thus, the use of weak promoter operably linked to a male-essential mitochondrial gene can produce mature seed that can produce a plant.

Examples of inducible promoters include the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (PCT US01/22169).

Examples of promoters under developmental control include promoters that initiate transcription preferentially in certain tissues, such as leaves, roots, fruit, seeds, or flowers. A tissue specific promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the recombinant DNA constructs comprise a tissue-preferred promoter. A tissue preferred promoter is a promoter that initiates transcription mostly, but not necessarily entirely or solely in certain tissues. For example, nucleic acid molecules encoding engineered meganuclease of the present disclosure can be operably linked to leaf-preferred, stem-preferred promoters, root-preferred promoters, or seed-preferred promoters. Tissue-preferred promoters (e.g., leaf-preferred promoters, stem-preferred promoters, root-preferred promoters, and/or seed-preferred promoters) can be utilized to target enhanced expression of an engineered meganuclease within a particular plant tissue. In specific embodiments, the promoter is a flower-specific or a flower preferred promoter. Promoters useful for expression of the polynucleotides disclosed herein can also be anther-specific or anther-preferred promoters. Tissue-preferred promoters are known in the art. See, for example, Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters and stem-preferred promoters are known in the art. See, for example, Yamamoto et al. (1997) Plant 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590. In addition, the promoters of cab and rubisco can also be used. See, for example, Simpson et al. (1958) EMBO J 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol Biol. 29(4):759-772); and roIB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179. The phaseolin gene (Murai et al. (1983) Science 23:476-482 and Sengupta-Gopalen et al. (1988) PNAS 82:3320-3324).

Seed-preferred promoters include both seed-specific promoters (those promoters active during seed development such as promoters of seed storage proteins) as well as seed-germinating promoters (those promoters active during seed germination). See Thompson et al. (1989) BioEssays 10:108. In some embodiments, the seed-preferred promoters have expression in embryo sac, early embryo, early endosperm, aleurone, and/or basal endosperm transfer cell layer (BETL). Seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed. Promoters that express in the embryo, pericarp, and endosperm are disclosed in U.S. Pat. No. 6,225,529 and PCT publication WO 00/12733. The disclosures for each of these are incorporated herein by reference in their entirety.

A “cell type-specific” or “tissue-specific” promoter is a promoter that primarily drives expression in certain cell types in one or more tissues, for example, anther tissues, pollen, flower, or vascular cells in roots, leaves, stalk cells, and stem cells. The expression cassette can also include cell type preferred promoters or tissue preferred promoters. A “cell type-preferred” promoter or “tissue-preferred” promoter is a promoter that primarily drives expression mostly, but not necessarily entirely or solely in certain cell types in one or more tissues, for example, anther tissues, pollen, flower, or vascular cells in roots, leaves, stalk cells, and stem cells.

Chemical-regulated promoters can be used to modulate the expression of a gene in a plant through the application of an exogenous chemical regulator. For example, chemical-regulated promoters can be used to modulate the expression of an engineered meganuclease in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

In some embodiments, an expression cassette described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

An expression cassette construct described herein may contain transfer DNA (T-DNA) sequences. For example, a recombinant DNA construct may contain T-DNA of tumor-inducing (Ti) plasmid of Agrobacterium tumefaciens. Alternatively, a recombinant DNA construct may contain T-DNA of tumor-inducing (Ti) plasmid of Agrobacterium rhizogenes. The vir genes of the Ti plasmid may help in transfer of T-DNA of an expression cassette into nuclear DNA genome of a host plant. For example, Ti plasmid of Agrobacterium tumefaciens may help in transfer of T-DNA of an expression cassette into nuclear DNA genome of a host plant, thus enabling the transfer of one or more polynucleotides of the present disclosure into nuclear DNA genome of a host plant.

Recombinant DNA constructs are provided that comprise any expression cassette disclosed herein. In particular embodiments, recombinant DNA constructs are provided that comprise expression cassettes having a polynucleotide with a nucleotide sequence encoding a MTEN or MTEM as disclosed herein. In some embodiments, recombinant DNA constructs are provided that comprise expression cassettes having a first polynucleotide with nucleic acid sequence encoding a first MTEN or MTEM and a second polynucleotide comprising a nucleic acid sequence encoding a second MTEN or MTEM. Also described herein is a plasmid containing an expression cassette or recombinant DNA construct of the present disclosure. For example, the present disclosure may provide a plasmid containing an expression cassette that comprises one or more polynucleotides, wherein each polynucleotide contains a nucleic acid sequence encoding an MTEN or MTEM described herein.

Bacteria containing an expression cassette or recombinant DNA construct of the present disclosure are provided herein. For example, the present disclosure provides an Agrobacterium tumefaciens containing an expression cassette or recombinant DNA construct that comprises one or more polynucleotides, wherein each polynucleotide contains a nucleic acid sequence encoding an MTEN or MTEM.

Recombinant viruses containing a recombinant DNA construct or expression cassette of the present disclosure are provided herein for the delivery of the expression cassette to a target cell. For example, the present disclosure may provide a recombinant virus containing a recombinant DNA construct that comprises one or more polynucleotides, wherein each polynucleotide contains a nucleic acid sequence encoding an engineered meganuclease described hereinabove. A recombinant virus described herein can be a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant bean yellow dwarf virus (BeYDV), or a recombinant turnip vein-clearing virus (TVCV). A recombinant virus described herein can be a recombinant AAV. The recombinant virus can be selected based on functionality in the target cell of interest. For example, recombinant viruses able to infect mammalian cells should be selected for mammalian cell delivery and recombinant viruses able to infect plant cells should be selected for plant cell delivery.

In some embodiments, a recombinant DNA construct contains a cis-acting regulatory element that is operably linked to a nucleic acid sequence encoding an engineered meganuclease described hereinabove. In certain embodiments, the cis-acting regulatory element directs the expression of the engineered meganuclease in a cell, such as in a eukaryotic cell. For example, a recombinant DNA construct may contain a cis-acting regulatory element that is operably linked to a nucleic acid sequence encoding an engineered meganuclease, wherein the cis-acting regulatory element may direct the expression of the engineered meganuclease in a cell, such as in a eukaryotic cell. In particular embodiments, the cis-acting regulatory element is a promoter. In specific embodiments, a recombinant DNA construct may contain a promoter sequence that is operably linked to a nucleic acid sequence encoding an engineered meganuclease described hereinabove. In certain instances, a recombinant DNA construct may contain a cassava vein mosaic virus (CSVMV) promoter that is operably linked to a nucleic acid sequence encoding an MTEM, wherein the promoter sequence may direct the expression of the engineered meganuclease in a cell, such as in a eukaryotic cell.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

A recombinant DNA construct of the present disclosure may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize Adh1 intron (Kyozuka, et al., (1991) Mol. Gen. Genet. 228:40-48; Kyozuka, et al., (1990) Maydica 35:353-357) and the like, herein incorporated by reference in their entirety.

In preparing the recombinant DNA constructs, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in the recombinant DNA constructs of the present invention. Examples of suitable reporter genes known in the art can be found in, for example, Jefferson, et al., (1991) in Plant Molecular Biology Manual, ed. Gelvin, et al. (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other polynucleotides that could be employed on the recombinant DNA constructs and expression cassettes disclosed herein include, but are not limited to, examples such as GUS (beta-glucuronidase; Jefferson, (1987) Plant Mol. Biol. Rep. 5:387), GFP (green fluorescence protein; Chalfie et al., (1994) Science 263:802), luciferase (Riggs et al., (1987) Nucleic Acids Res. 15(19):8115 and Luehrsen et al., (1992) Methods Enzymol. 216:397-414) and the maize genes encoding for anthocyanin production (Ludwig et al., (1990) Science 247:449), herein incorporated by reference in their entirety.

A recombinant DNA construct and/or expression cassette of the present disclosure can include an additional polynucleotide encoding herbicide or antibiotic resistance traits including genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene; see, for example, U.S. Publication No. 20040082770 and WO 03/092360); or other such genes known in the art. The bar gene encodes resistance to the herbicide basta, the nptII gene encodes resistance to the antibiotics kanamycin and geneticin, the hptII gene encodes resistance to the antibiotic hygromycin, and the ALS-gene mutants encode resistance to the herbicide chlorsulfuron. Additional herbicide resistance traits are described for example in U.S. Patent Application 2016/0208243, herein incorporated by reference.

In still other embodiments, the recombinant DNA construct and/or expression cassette can include an additional polynucleotide encoding an agronomically important trait, such as a plant hormone, plant defense protein, a nutrient transport protein, a biotic association protein, a desirable input trait, a desirable output trait, a stress resistance gene, a disease/pathogen resistance gene, a male sterility, a developmental gene, a regulatory gene, a DNA repair gene, a transcriptional regulatory gene or any other polynucleotide and/or polypeptide of interest. For example, the recombinant DNA construct can include an additional polynucleotide encoding sterility genes that provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development. Additional sterility traits are described for example in U.S. Patent Application 2016/0208243, herein incorporated by reference. In some embodiments, the recombinant DNA construct and/or expression cassette can include additional polynucleotides that downregulate the expression of genes responsible for agronomically important traits. In specific embodiments, the recombinant DNA construct and/or expression constructs disclosed herein can comprise a polynucleotide having a nucleic acid sequence encoding a male essential gene. In some embodiments, the male essential gene is an mtATPase subunit, such as mtATP1.

In some instances, a recombinant DNA construct can include an additional polynucleotide encoding traits, such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389. In some instances, a recombinant DNA construct of the present disclosure can include an additional polynucleotide encoding commercial traits. For example, in some embodiments, a recombinant DNA construct disclosed herein can comprise a nucleic acid sequence that can increase for example, starch for ethanol production, or provide expression of proteins. A recombinant DNA construct can also include an additional polynucleotide encoding traits that are useful in production of polymers and bioplastics such as described in U.S. Pat. No. 5,602,321. Genes such as β-Ketothiolase, PHBase (polyhydroxybutyrate synthase), and acetoacetyl-CoA reductase (see Schubert et al. (1988) J. Bacterial. 170:5837-5847) facilitate expression of polyhydroxyalkanoates (PHAs).

In some instances, a recombinant DNA construct includes additional polynucleotides encoding desirable plant traits. Such traits include, for example, disease resistance, herbicide tolerance, drought tolerance, salt tolerance, insect resistance, resistance against parasitic weeds, improved plant nutritional value, improved forage digestibility, increased grain yield, cytoplasmic male sterility, altered fruit ripening, increased storage life of plants or plant parts, reduced allergen production, and increased or decreased lignin content. Genes capable of conferring these desirable traits are disclosed in U.S. Patent Application 2016/0208243, herein incorporated by reference.

### 2.4 Methods for Producing Genetically-Modified Cells

The invention provides methods for producing genetically-modified cells using engineered nucleases that bind and cleave recognition sequences found within mtDNA. For example, provided herein are methods for producing genetically-modified cells using engineered nucleases that bind and cleave recognition sequences, such as ATP 5-6 recognition sequence (SEQ ID NO: 1) or ATP 7-8 recognition sequence (SEQ ID NO: 2) found within a mitochondrial ATP synthase complex (mtATP) gene. Cleavage at such recognition sequences can allow for insertion of an exogenous sequence via homologous recombination, degradation of the mtDNA, or in some cases, ligation of generated cleavage sites to one another.

In some embodiments, provided herein are methods for producing a genetically-modified eukaryotic cell by introducing into the eukaryotic cell a polynucleotide of the present disclosure, such as a polynucleotide containing a nucleic acid sequence that encodes an MTEN or MTEM described hereinabove. Upon expression in the eukaryotic cell, the MTEN or MTEM localizes to the mitochondria, binds a recognition sequence in the mitochondrial genome, and generates a cleavage site. The cleavage site generated by the MTEN or MTEM can be repaired by non-homologous end joining (NHEJ) repair pathway which may result in a nucleic acid insertion or deletion at the cleavage site. In some embodiments, cleavage of multiple recognition sequences and subsequent ligation of the cleaved ends of the mitochondrial genome can produce a deletion in a gene of the mitochondrial genome. In certain instances, the recognition sequence is within a region of the mitochondrial genome associated with a plant mitochondrial disorder. Alternatively, the recognition sequence can be within a region of the mitochondrial genome associated with a mitochondrial trait. For example, the recognition sequence can be within a region of the mitochondrial genome associated with the Cytoplasmic Male Sterility (CMS) system.

In some embodiments, provided herein are methods for producing a genetically-modified eukaryotic cell by introducing into the eukaryotic cell a first MTEN/MTEM or first polynucleotide containing a nucleic acid sequence encoding a first MTEN/MTEM (e.g., MTEM comprising an ATP 5-6 nuclease) and a second MTEN/MTEM or a polynucleotide containing a nucleic acid sequence encoding a second MTENMTEM (e.g., MTEM comprising an ATP 7-8 nuclease). Upon expression in a eukaryotic cell and localization to the mitochondria, the first MTEN/MTEM may bind and cleave a first recognition sequence (e.g., ATP 5-6 recognition sequence; SEQ ID NO: 1) in the mitochondrial genome of the eukaryotic cell to generate a first cleavage site. Moreover, upon expression in the eukaryotic cell and localization to the mitochondria, the second MTEN/MTEM may bind and cleave a second recognition sequence (e.g., ATP 7-8 recognition sequence; SEQ ID NO: 2) in the mitochondrial genome of the eukaryotic cell to generate a second cleavage site. The first cleavage site and the second cleavage site may have complementary overhangs. In some embodiments, the cleavage sites generated by the first MTEN/MTEM and the second MTEN/MTEM in the mitochondrial genome of the eukaryotic cell is repaired by NHEJ. The cleavage sites generated by the first engineered meganuclease and the second engineered meganuclease in the mitochondrial genome of the eukaryotic cell can also be repaired by alternative nonhomologous end-joining (Alt-NHEJ) or microhomology-mediated end joining (MMEJ). In certain embodiments, the NHEJ or Alt-NHEJ/MMEJ results in insertion and/or deletion of a nucleic acid at the cleavage site. In particular embodiments, the NHEJ or Alt-NHEJ/MMEJ results in insertion and/or deletion of at least 200 (e.g., at least 200, 225, 250, 275, 300, 325, 350, 375, 400, 425, 450, 475, 500, 525, 550, 575, 600, 625, 650, 675, 700, 725, 750, 775, 800, or more) nucleotides at the cleavage site. In particular embodiments, the first and second cleavage sites ligate to one another, in some instances by ligation of complementary overhangs, to anneal the gene after cleavage.

In some embodiments, the first recognition sequence and the second recognition sequence are within a same gene. In particular embodiments, the distance between the first recognition sequence and the second recognition sequence in the gene is at least 200 (e.g., at least 200, 225, 250, 275, 300, 325, 350, 375, 400, 425, 450, 475, 500, 525, 550, 575, 600, 625, 650, 675, 700, 725, 750, 775, 800, or more) nucleotides. For example, the first recognition sequence and the second recognition sequence may be within a mitochondrial ATP synthase complex (mtATP) gene. In particular, the distance between the first recognition sequence and the second recognition sequence in the mtATP gene can be at least 200 nucleotides.

Methods are provided for expression of an engineered meganuclease in a cell by introducing into the cell an expression cassette of the present disclosure that contains a nucleic acid sequence encoding an MTEN/MTEM. The cell may be a eukaryotic or a prokaryotic cell. For example, the present disclosure may provide methods for expression of an engineered meganuclease in a eukaryotic cell by introducing into the eukaryotic cell an expression cassette that contains a nucleic acid sequence encoding MTEN/MTEM disclosed herein or encoding two MTENs/MTEMs disclosed herein. The eukaryotic cell may be plant cell or an animal cell. The plant cell can be any plant cell disclosed herein and in particular embodiments, the plant cell is a tobacco cell, rice cell, maize cell, sugarcane cell, sorghum cell, millet cell, switchgrass cell, alfalfa cell, silage corn cell, hay cell, Miscanthus sp. cell, cotton cell, tomato cell, blackberry cell, raspberry cell, cucumber cell, watermelon cell, grape cell, pomegranate cell, or pepper cell. The animal cell may be a mammalian cell, such as a human cell.

The terms “introducing” and “introduced” are intended to mean providing a nucleic acid (e.g., a recombinant DNA construct containing a polynucleotide of the present disclosure) or protein (e.g., an engineered meganuclease of the present disclosure) into a cell. The term “introduced” includes reference to the incorporation of a nucleic acid into a eukaryotic or prokaryotic cell, where the nucleic acid may be incorporated into the genome of the cell, and also includes reference to the transient provision of a nucleic acid or protein to the cell. The term “introduced” further includes reference to stable or transient transformation methods, as well as sexually crossing. Thus, “introduced” in the context of inserting a nucleic acid (e.g., a recombinant DNA construct) into a cell, means “transfection” or “transformation” or “transduction” and includes the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed.

“Stable transformation” is intended to mean that a polynucleotide introduced into a host cell (e.g., a eukaryotic cell) integrates into the genome of the host and is capable of being inherited by the progeny thereof. “Transient transformation” is intended to mean that a polynucleotide is introduced into the host cell (e.g., a eukaryotic cell) and expressed temporally.

### 2.5 Transformation of Plants

Provided herein are compositions and methods for transformation of plants with expression cassettes described hereinabove, such as expression cassettes containing nucleic acid sequences encoding MTEMs or MTENs of the present disclosure. While the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced expression cassettes. In some embodiments, the nuclear genome of the plant or plant part comprises a maintainer construct. In certain embodiments, the nuclear genome of the plant or plant part comprises a restorer construct.

Methods for transformation involve introducing expression cassettes containing a polynucleotide of the present disclosure into a plant. By “introducing” is meant a method of introducing the polynucleotide to the plant or other host cell in such a manner that the polynucleotide gains access to the interior of a cell of the plant or host cell. The methods of the present disclosure do not require a particular method for introducing a polynucleotide to a plant or host cell, only that the polynucleotide gains access to the interior of at least one cell of the plant or the host organism. Methods for introducing polynucleotides into plants and other host cells are known in the art including, but not limited to, stable transformation methods, transient transformation methods, and virus-mediated methods.

Transformation protocols as well as protocols for introducing polynucleotide sequences into plants cells are well established. Suitable methods of introducing polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050) herein incorporated by reference.

In some embodiments, a recombinant DNA construct containing an expression cassette of the present disclosure is provided to a plant using a variety of transient transformation methods. Such transient transformation methods include, but are not limited to, the introduction of the polynucleotide directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, the polynucleotides can be transiently transformed into the plant using a viral vector system or the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylamine (PEI; Sigma #P3143).

In other embodiments, expression cassettes disclosed herein may be introduced into plants, plant parts, or plant cells by contacting the plants, plant parts or plant cells with a virus or viral nucleic acids. Generally, such methods involve incorporating a recombinant DNA construct provided herein within a viral DNA or RNA molecule. Methods for introducing polynucleotides into plants, plant parts, or plant cells and expressing a protein (e.g., an engineered meganuclease) encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the genome of a plant or plant part. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the recombinant DNA constructs comprising a polynucleotide of the present disclosure can be contained in a transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The recombinant DNA construct is thereby integrated at a specific chromosomal position in the plant genome.

Any method can be used to introduce the expression cassettes disclosed herein into a plant or plant cell for expression of an MTEN/MTEM disclosed herein. For example, precise genome-editing technologies can be used to introduce the recombinant DNA constructs disclosed herein into the plant genome. In this manner, a polynucleotide can be inserted proximal to a native plant sequence through the use of methods available in the art. Such methods include, but are not limited to, meganucleases designed against the plant genomic sequence of interest (D'Halluin et al (2013) Plant Biotechnol J 11: 933-941); CRISPR-Cas9, TALENs, and other technologies for precise editing of genomes (Feng, et al. Cell Research 23:1229-1232, 2013, Podevin, et al. Trends Biotechnology 31: 375-383, 2013, Wei et al. 2013 J Gen Genomics 40: 281-289, Zhang et al 2013, WO 2013/026740); Cre-lox site-specific recombination (Dale et al. (1995) Plant J7:649-659; Lyznik, et al. (2007) Transgenic Plant J 1:1-9; FLP-FRT recombination (Li et al. (2009) Plant Physiol 151:1087-1095); Bxb1-mediated integration (Yau et al. Plant J (2011) 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65); Puchta, H. (2002) Plant Mol Biol 48:173-182).

There are various methods of introducing heterologous polynucleotide sequences into both monocotyledonous and dicotyledonous plants (Potrykus et al., Annu Rev Plant Physiol Plant Mol Biol 42:205-225 (1991); Shimamoto et al., Nature 338:274-276 (1989)). The principle methods of causing stable integration of heterologous polynucleotide sequences into plant genomic DNA include two main approaches:
(i) Agrobacterium-mediated gene transfer: Klee et al., Annu Rev Plant Physiol 38:467-486 (1987); Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

Methods of direct DNA transfer into plant cells are also provided herein. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

In specific embodiments, an expression cassette, such as an expression cassette containing a nucleic acid sequence encoding an MTEN/MTEM of the present disclosure, is introduced into a plant cell by Agrobacterium-mediated transformation, biolistic transformation, microinjection, or electroporation. Also provided herein are plants, plant parts, or plant cells containing an expression cassette of the present disclosure, such as an expression cassette containing a nucleic acid sequence encoding an MTEN/MTEM described hereinabove. Seeds produced from the plants described hereinabove are also provided herein, such that the seeds contain an expression cassette of the present disclosure, such as an expression cassette containing a nucleic acid sequence encoding an MTEN/MTEM described hereinabove.

In some embodiments, the plant is tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper. For example, the present invention may provide tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper containing an expression cassette described hereinabove, such as an expression cassette containing a nucleic acid sequence encoding an MTEN/MTEM. The present disclosure also provides seed produced from the genetically-modified plant (e.g., tobacco, rice, maize, sugarcane, sorghum, millet, switchgrass, alfalfa, silage corn, hay, Miscanthus sp., cotton, tomato, or pepper), wherein the seed contains an expression cassette described hereinabove, such as an expression cassette containing a nucleic acid sequence encoding an MTEN/MTEM. Plant parts of tobacco, rice, maize, sugarcane, sorghum, millet, switchgrass, alfalfa, silage corn, hay, Miscanthus sp., cotton, tomato, or pepper containing an expression cassette described hereinabove, such as an expression cassette containing a nucleic acid sequence encoding an MTEN/MTEM. The present invention may also provide cell of tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper containing an expression cassette described hereinabove, such as an expression cassette containing a nucleic acid sequence encoding an MTEN/MTEM. In some embodiments, the nuclear genome of the plant comprises a maintainer construct. In certain embodiments, the nuclear genome of the plant comprises a restorer construct. In certain embodiments, a tomato, blackberry, raspberry, cucumber, watermelon, pomegranate, grape, or pepper plant is provided that lacks a mature seed. In some embodiments a seedless fruit is provided from a tomato, blackberry, raspberry, cucumber, watermelon, pomegranate, grape, or pepper.

In specific embodiments, a first expression cassette and/or a second expression cassette are introduced into a plant cell by Agrobacterium-mediated transformation, biolistic transformation, microinjection or electroporation, wherein the first expression cassette contains a nucleic acid sequence encoding a first MTEN/MTEM, and the second polynucleotide contains a nucleic acid sequence encoding a second MTEN/MTEM. Also provided herein are plants, plant parts or plant cells containing a first expression cassette and a second expression cassette, wherein the first expression cassette contains a nucleic acid sequence encoding a first MTEN/MTEM, and the second polynucleotide contains a nucleic acid sequence encoding a second MTEN/MTEM. Also provided herein are seeds produced from the plants described hereinabove, such that the seeds contain a first expression cassette and a second expression cassette, wherein the first expression cassette contains a nucleic acid sequence encoding a first MTEN/MTEM, and the second polynucleotide contains a nucleic acid sequence encoding a second MTEN/MTEM. In some embodiments, the plant is tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper. For example, the present invention may provide tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper. Plant parts of tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper are provided herein containing a first expression cassette and a second expression cassette, wherein the first expression cassette contains a nucleic acid sequence encoding a first MTEN/MTEM, and the second polynucleotide contains a nucleic acid sequence encoding a second MTEN/MTEM. A cell of tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper plant are provided herein containing a first expression cassette and a second expression cassette, wherein the first expression cassette contains a nucleic acid sequence encoding a first MTEN/MTEM, and the second polynucleotide contains a nucleic acid sequence encoding a second MTEN/MTEM.

Following stable transformation, the transformed plant or plant part can be propagated to grow the plant or plant part into a mature plant. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Thus, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Transient transformation of leaf cells, meristematic cells or the whole plant is provided herein. Transient transformation can be effected by any of the direct DNA transfer methods described above or by viral infection using modified plant viruses.

Viruses that have been shown to be useful for the transformation of plant hosts include CaMV, TMV, and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261. A recombinant virus useful in the composition and methods of the present disclosure may include a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76. When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein, which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA. Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

According to some embodiments of the invention, there is provided a host cell heterologously expressing an expression cassette or recombinant DNA construct of the invention, as described hereinabove. The host cell can be any suitable host cell include bacteria, yeast and other microorganisms that can be cultured or grown in fermentation, plant and other eukaryotic cells. For example, the host cell can be a bacterial cell (e.g., E. coli and B. subtilis) transformed with a heterologous nucleic acid, such as bacteriophage DNA, plasmid DNA, or cosmid DNA expression vectors containing the polynucleotide or nucleic acid molecules described herein, or yeast (e.g., S. cerevisiae or S. pombe) transformed with recombinant yeast expression vectors containing the polynucleotide or nucleic acid molecules described herein. In some embodiments, the host cell is a plant cell. For example, the host cell can be a plant cell, such as tobacco cell, rice cell, maize cell, sugarcane cell, sorghum cell, millet cell, switchgrass cell, alfalfa cell, silage corn cell, hay cell, Miscanthus sp. cell, cotton cell, tomato cell, blackberry cell, raspberry cell, cucumber cell, watermelon cell, grape cell, pomegranate cell, or pepper cell. In some embodiments, the host cell is a yeast cell.

The methods disclosed herein may also employ a mixture of recombinant and non-recombinant host. If more than one host is used then the hosts may be co-cultivated, or they may be cultured separately. If the hosts are cultivated separately the intermediate products may be recovered and optionally purified and partially purified and fed to recombinant hosts using the intermediate products as substrates.

Recombinant hosts described herein can be used in methods to express an MTEN/MTEM of the present disclosure. For example, if the recombinant host is a microorganism, the method can include growing the recombinant microorganism in a culture medium under conditions in which one or more of the engineered meganucleases of the invention are expressed. The recombinant microorganism may be grown in a fed batch or continuous process. Typically, the recombinant microorganism is grown in a fermenter at a defined temperature(s) for a desired period of time. A cell lysate can be prepared from the recombinant host expressing one or more engineered meganucleases.

The methods disclosed herein can result in a genetically-modified organism comprising an expression cassette with a nucleic acid sequence encoding an MTEN/MTEM disclosed herein. In specific embodiments, the organism is a plant, including whole plants, as well as plant parts or plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). In specific embodiments, a genetically-modified plant cell produced by the methods of the present disclosure may contain a first expression cassette and a second expression cassette, wherein the first expression cassette contains a nucleic acid sequence encoding a first MTEN/MTEM, and the second polynucleotide contains a nucleic acid sequence encoding a second MTEN/MTEM.

As used herein, the term “genetically modified” or “transgenic” or “transformed” or “stably transformed” plants, plant cells, plant tissues, plant parts or seeds refers plants, plant cells, plant tissues, plant parts or seeds that have been modified by the methods of the present disclosure to contain: (i) an expression cassette, such as an expression cassette that contains a nucleic acid sequence encoding an MTEN/MTEM; and/or (ii) a first expression cassette and a second expression cassette, wherein the first expression cassette contains a nucleic acid sequence encoding a first MTEN/MTEM, and the second expression cassette contains a nucleic acid sequence encoding a second MTEN/MTEM. In contrast, control, non-transgenic, or unmodified plants, plant cells, plant tissues, plant parts or seeds refer to plants, plant cells, plant tissues, plant parts or seeds that are without such modifications (e.g., do not contain a polynucleotide of the present disclosure). It is recognized that other exogenous or endogenous nucleic acid sequences or DNA fragments may also be incorporated into the plant cell.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, a polynucleotide of the invention, stably incorporated into their genome. In specific embodiments, the sequences provide herein can be targeted to specific site within the genome of the host cell or plant cell. Methods for targeting sequence to specific sites in the genome can include the use of engineered nucleases.

The present invention may be used for transformation of any plant species, including, but not limited to, monocots and dicots (i.e., monocotyledonous and dicotyledonous, respectively). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), pepper (Capsicum annuum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. In certain embodiments, the present invention is used for transformation of tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper plant. In specific embodiments, an expression cassette comprising a nucleic acid sequence encoding an MTEN/MTEM as described herein can be introduced into a plant, plant part, or plant cell. Subsequently, a plant or plant part having the introduced expression cassette of the invention is selected using methods known to those of skill in the art, such as, but not limited to, Southern blot analysis, DNA sequencing, PCR analysis, or phenotypic analysis. A plant or plant part genetically-modified by the foregoing embodiments is grown under plant forming conditions for a time sufficient to modulate the concentration and/or activity of the MTEN/MTEM described herein expressed by the plant. Plant forming conditions are well known in the art and discussed briefly elsewhere herein.

According to the present invention, a control plant or plant part may comprise a wild-type plant or plant part, i.e., of the same genotype as the starting material for the genetic alteration that resulted in the subject plant or plant part. A control plant or plant part may also comprise a plant or plant part of the same genotype as the starting material but that has been transformed with a null construct (i.e., with a construct that does not contain the polynucleotide of the present disclosure). Finally, a control plant or plant part may comprise the subject plant or plant part itself under conditions in which the MTEN/MTEM is not expressed. In all such cases, the subject plant or plant part and the control plant or plant part are cultured and harvested using the same protocols.

### 2.6 Cytoplasmic Male Sterility

In some embodiments, mitochondrial genome editing by the compositions and methods of the present disclosure can be used in a Cytoplasmic Male Sterility (CMS) system to produce hybrid seeds. In the CMS system, using an MTEN/MTEM or a polynucleotide (e.g., a polynucleotide that contains a nucleic acid sequence encoding an MTEN/MTEM) of the present disclosure, a mitochondrial male-essential plant gene can be inactivated to produce a male sterile plant when introduced into a plant in which a nuclear version of that gene has been engineered in a manner where it is expressed in all tissues of the plant except the male tissues (anthers). In certain embodiments, the mitochondrial male-essential gene is a gene encoding a subunit of the mitochondrial ATP synthase (i.e., ATP synthase gene). In particular embodiments, the mitochondrial male-essential gene is at least one of: ATP1, ATP4, ATP6, ATP8, ATP9, COXI, COXII, COXIII, COB, NAD1, NAD5, or active fragments or variants thereof. In specific embodiments, a fragment of a mitochondrial male-essential gene can be deleted in order to inactive the gene and reduce male fertility.

A “restorer gene” is created by transferring the targeted mitochondrial male-essential gene to the nuclear genome, and a “maintainer gene” is developed by placing the transferred mitochondrial male-essential gene under the control of a promoter that is expressed everywhere in the plant except male tissues, such as developing anther tissue.

In order to create a male sterile plant, any mitochondrial male-essential gene can be targeted for inactivation. In specific embodiments, the mitochondrial gene targeted by the MTEN/MTEM disclosed herein for the purpose of eliminating function is that encoding the α-subunit of the mitochondrial ATP synthase complex (gene designated mtATP1), which is coordinately regulated with the β-subunit of the mtATPase which is a nuclear gene (designated ATP2). Therefore, the utilization of the regulatory sequences normally associated with ATP2 (including its transit peptide) may enable proper restoration of mtATP1 gene function when expressed from the nucleus.

In specific embodiments, before a mitochondrial male-essential gene of a plant cell or plant part is cleaved by a MTEN/MTEM disclosed herein, the plant cell or plant part comprises a nuclear copy of the targeted mitochondrial male-essential gene.

Thus, in specific embodiments, a “restorer construct” is provided that includes a male-essential mitochondrial gene operably linked to a ubiquitous promoter in order to restore function of the inactivated male-essential mitochondrial gene. In some embodiments, the male-essential mitochondrial gene encodes mtATP1. The male-essential mitochondrial gene on the restorer construct can be codon-optimized for expression in the nucleus of the plant of interest. Moreover, in some embodiments, the male-essential mitochondrial gene on the restorer construct is codon-optimized for expression in the nucleus and to remove a recognition sequence of an MTEN/MTEM disclosed herein. The ubiquitous promoter of the restorer construct can be any ubiquitous promoter, and in specific embodiments is the promoter from a mitochondrial ATPase gene (“mtATP promoter”) such as the β-ATP promoter. In some embodiments for producing a plant without mature seeds, a weak ubiquitous promoter can be used. Strong ubiquitous promoters can be used to produce mature hybrid seed capable of developing into a plant. A “restorer plant” comprises a restorer construct on the nuclear genome and has wild-type version of the male-essential gene on the mitochondrial genome.

In some embodiments, the restorer construct comprises a nucleic acid sequence encoding a restorer MTP attached the protein product of the male-essential gene expressed from the restorer construct. In specific embodiments, the restorer MTP is from the β-ATP protein having the amino acid sequence set forth in SEQ ID NO: 7 or 8. in some embodiments, the MTP has an amino acid sequence with at least 80%, 85%, 90%, 91%, 92%, 93%, 94%, 95%, 96%, 97%, 98%, 99%, or 100% sequence identity to any one of SEQ ID NOs: 7-11. In specific embodiments, the MTP can be attached to the N-terminus of the male-essential gene. In other embodiments the MTP can be attached to the C-terminus of the male-essential gene. In specific embodiments, the MTP is attached by fusing the MTP to the N- or C-terminus of the male-essential gene. The MTP can also be attached to the male-essential gene by a peptide linker. The linker can be, for example, about 1, 2, 3, 4, 5, 6, 7, 8, 9, 10, 11, 12, 15, or 20 amino acids. In specific embodiments the MTP is attached to a peptide linker at the N- or C-terminus of the male-essential gene.

In some embodiments a “maintainer construct” is provided that comprises a male-essential mitochondrial gene operably linked to a non-male promoter. As used herein, a “non-male promoter” is active in all tissues except male tissues. In specific examples, the non-male promoter is not active in the anther or pollen of a plant or plant part. In some embodiments, the male-essential mitochondrial gene encodes mtATP1. The male-essential mitochondrial gene on the maintainer construct can be codon-optimized for expression in the nucleus of the plant of interest. Moreover, in some embodiments, the male-essential mitochondrial gene on the maintainer construct is codon-optimized for expression in the nucleus and to remove at least one recognition sequence of an MTEN/MTEM disclose herein. The non-male of the restorer construct can be any non-male promoter, and in specific embodiments is the CaMV35S promoter or enhanced CaMV35S promoter. In some embodiments the non-male promoter is a strong non-male promoter. In certain embodiments, the non-male promoter is a weak non-male promoter. The promoter can be a ubiquitous promoter. In some embodiments, the ubiquitous promoter is a weak ubiquitous promoter. In some embodiments, the ubiquitous promoter is an mtATP promoter. In some embodiments, the ubiquitous promoter is a β-ATP promoter. In some embodiments, the ubiquitous promoter is a strong ubiquitous promoter. In some embodiments, the strong ubiquitous promoter is a ubiquitin promoter.

In some embodiments, the maintainer construct comprises a nucleic acid sequence encoding a maintainer MTP attached the protein product of the male-essential gene expressed from the maintainer construct. In specific embodiments, the maintainer MTP is from the β-ATP protein having the amino acid sequence set forth in SEQ ID NO: 7 or 8. in some embodiments, the maintainer MTP has an amino acid sequence with at least 80%, 85%, 90%, 91%, 92%, 93%, 94%, 95%, 96%, 97%, 98%, 99%, or 100% sequence identity to any one of SEQ ID NOs: 7-11. In specific embodiments, the MTP can be attached to the N-terminus of the male-essential gene. In other embodiments the MTP can be attached to the C-terminus of the male-essential gene. In specific embodiments, the MTP is attached by fusing the MTP to the N- or C-terminus of the male-essential gene product. The MTP can also be attached to the male-essential gene by a peptide linker. The linker can be, for example, about 1, 2, 3, 4, 5, 6, 7, 8, 9, 10, 11, 12, 15, or 20 amino acids. In specific embodiments the MTP is attached to a peptide linker at the N- or C-terminus of the male-essential gene product.

Accordingly, in order to create a male-sterile plant, an MTEN/MTEM or an expression cassette comprising a nucleic acid sequence encoding an MTEN/MTEM can be introduced into a plant or plant part by any methods known in the art or disclosed herein. In specific embodiments, the nuclear genome of the plant or plant part into which the MTEN/MTEM or expression cassette comprising a nucleic acid sequence encoding an MTEN/MTEM is introduced comprises a maintainer construct. Upon expression of the MTEN/MTEM disclosed herein, the recognition sequence in the male-essential mitochondrial gene is cleaved and the male-essential mitochondrial gene is inactivated. In some embodiments a first MTEN/MTEM and a second MTEN/MTEM cleave a respective first and second recognition sequence within a male-essential mitochondrial gene of a plant or plant part comprising a maintainer construct having a nuclear copy of the same male-essential gene. Following cleavage, then ends of the mitochondrial genome can ligate resulting in a deletion and inactivation of the male-essential mitochondrial gene or the essential mitochondrial gene is deleted, either all or in part, presumably via homologous recombination across short regions or microhomology within the mitochondrial genome. The resulting male sterile plant is cultured (i.e., grown) and the nuclear copy of the male-essential gene will be expressed throughout the plant, but not in the male tissues. This male-sterile plant on a given variety (e.g., Inbred A) having a modified mitochondrial genome can serve as the female parent in crosses with an isogenic inbred plant of the same variety (e.g., Inbred A) that possesses the same maintainer construct on the nuclear chromosome but whose cytoplasm is normal, in order to propagate the male-sterile trait without the requirement for further introduction of an MTEN/MTEM or expression cassette having a nucleic acid sequence encoding an MTEN/MTEM. Preferably, after one or two generations of crossing to the maintainer line, the MTEN/MTEM construct will have been lost through segregation.

A male-sterile plant of a given variety (e.g., “Inbred A”) having a maintainer construct can be crossed with a restorer plant of a different variety (e.g., “Inbred B”) in order to produce hybrid seed of the crossed variety (e.g., Hybrid A×B). The genetically diverse inbred varieties can produce a hybrid seed with heterosis and hybrid vigor. The resulting hybrid seed will comprise the inactive male-essential mitochondrial gene because mitochondrial genomes are only inherited from the female parent. Any plant can be used in the CMS system described herein. In particular embodiments, the plant is tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper. Accordingly, provided herein is a genetically modified hybrid seed created by the CMS systems disclosed herein and genetically-modified hybrid plants produced by growing the hybrid seed.

### 2.7 Breeding of Plants

Also disclosed herein are methods for breeding a plant, such as a tobacco, rice, maize, sugarcane, sorghum, millet, switchgrass, alfalfa, silage corn, hay, Miscanthus sp., cotton, tomato, blackberry, raspberry, cucumber, watermelon, pomegranate, grape, or pepper that has been modified to contain one or more expression cassettes of the present disclosure. A plant containing one or more expression cassettes of the present disclosure may be regenerated from a plant cell or plant part, wherein the genome of the plant cell or plant part is genetically-modified to contain one or more expression cassettes of the present disclosure. Using conventional breeding techniques, including micropropagation, vegetative cuttings, cross-pollination or self-pollination (in the case of restorer or maintainer lines), one or more seeds may be produced from the plant that contains an expression cassette of the present disclosure. Such a seed, and the resulting progeny plant grown from such a seed, may contain one or more expression cassettes of the present disclosure, and therefore may be transgenic. Progeny plants are plants having a genetic modification to contain one or more expression cassettes of the present disclosure, which descended from the original plant having modification to contain one or more expression cassettes of the present disclosure.

Seeds, micropropagations, or vegetative cuttings produced using such a plant of the invention can be harvested and used to grow generations of plants having genetic modification to contain one or more expression cassettes of the present disclosure, e.g., progeny plants, of the invention, comprising the one or more expression cassettes and optionally expressing a gene of agronomic interest (e.g., herbicide resistance gene). Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

### 2.8 Variants

The present invention encompasses variants of the polypeptide and polynucleotide sequences described herein. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; for example, the ability to bind and cleave recognition sequences found in mitochondrial ATP synthase complex (mtATP) gene, such as ATP 5-6 recognition sequence (SEQ ID NO: 1) and/or ATP 7-8 recognition sequence (SEQ ID NO: 2). Such variants may result, for example, from human manipulation. In some embodiments, biologically active variants of a native polypeptide (e.g., SEQ ID NO: 3 or 5) of the embodiments, or biologically active variants of the recognition half-site binding subunits described herein, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, native subunit, native HVR1, or native HVR2 as determined by sequence alignment programs and parameters described elsewhere herein. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, engineered meganucleases of the invention can comprise variants of the HVR1 and HVR2 regions disclosed herein. Parental HVR regions can comprise, for example, residues 24-79 or residues 215-270 of the exemplified engineered meganucleases. Thus, variant HVRs can comprise an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to an amino acid sequence corresponding to residues 24-79 or residues 215-270 of the engineered meganucleases exemplified herein, such that the variant HVR regions maintain the biological activity of the engineered meganuclease (i.e., binding to and cleaving the recognition sequence). Further, in some embodiments of the invention, a variant HVR1 region or variant HVR2 region can comprise residues corresponding to the amino acid residues found at specific positions within the parental HVR. In this context, “corresponding to” means that an amino acid residue in the variant HVR is the same amino acid residue (i.e., a separate identical residue) present in the parental HVR sequence in the same relative position (i.e., in relation to the remaining amino acids in the parent sequence). By way of example, if a parental HVR sequence comprises a serine residue at position 26, a variant HVR that “comprises a residue corresponding to” residue 26 will also comprise a serine at a position that is relative (i.e., corresponding) to parental position 26.

In particular embodiments, engineered meganucleases of the invention comprise an HVR1 that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 215-270 of SEQ ID NO: 3 or 5.

In certain embodiments, engineered meganucleases of the invention comprise an HVR2 that has 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 24-79 of SEQ ID NO: 3 or 5.

A substantial number of amino acid modifications to the DNA recognition domain of the wild-type I-CreI meganuclease have previously been identified (e.g., U.S. Pat. No. 8,021,867) which, singly or in combination, result in engineered meganucleases with specificities altered at individual bases within the DNA recognition sequence half-site, such that the resulting rationally-designed meganucleases have half-site specificities different from the wild-type enzyme. Table 1 provides potential substitutions that can be made in an engineered meganuclease monomer or subunit to enhance specificity based on the base present at each half-site position (−1 through −9) of a recognition half-site. Such substitutions are incorporated into variants of the meganucleases disclosed herein.

TABLE 1
Potential substitutions in engineered meganuclease variants

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | T44* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

Bold entries are wild-type contact residues and do not constitute “modifications” as used herein. An asterisk indicates that the residue contacts the base on the antisense strand.

Certain modifications can be made in an engineered meganuclease monomer or subunit to modulate DNA-binding affinity and/or activity. For example, an engineered meganuclease monomer or subunit described herein can comprise a G, S, or A at a residue corresponding to position 19 of I-CreI or SEQ ID NO: 3 or 5 (WO 2009001159), a Y, R, K, or D at a residue corresponding to position 66 of I-CreI or SEQ ID NO: 3 or 5 and/or an E, Q, or K at a residue corresponding to position 80 of I-CreI or SEQ ID NO: 3 or 5 (U.S. Pat. No. 8,021,867).

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode an engineered meganuclease, or an exogenous nucleic acid molecule, or template nucleic acid of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its ability to preferentially bind and cleave recognition sequences found within mitochondrial ATP synthase complex (mtATP) gene, such ATP 5-6 recognition sequence (SEQ ID NO: 1) and ATP 7-8 recognition sequence (SEQ ID NO: 2).

TABLE of the Sequences

| SEQ ID NO: | SEQUENCE | DESCRIPTION |
|---|---|---|
| 1 | GATGGGATTGCACGTGTTTATG | ATP 5-6 recognition sequence |
| | | (sense) |
| 2 | TAATGGAATGCACGCATTAATA | ATP 7-8 recognition sequence |
| | | (sense) |
| 3 | MNTKYNKEFLLYLAGFVDGDGSITACIQPG | ATP 5-6x.87 meganuclease amino |
| | QHCKFKHVLRLRFGVSQKTQRRWFLDKLVD | acid sequence |
| | EIGVGYVYDLGSVSEYRLSQIKPLHNELTQ | |
| | LQPFLKLKQKQANLVLKIIEQLPSAKESPD | |
| | KFLEVCTWVDQIAALNDSKTRKTTSETVRA | |
| | VLDSLPGSVGGLSPSQASSAASSASSSPGS | |
| | GISEALRAGAGSGTGYNKEFLLYLAGEVDS | |
| | DGSIFAAILPQQPRKFKHALQLFFVVTQKT | |
| | QRRWFLDKLVDEIGVGYVVDRGSVSDYKLS | |
| | QIKPLHNFLTQLQPFLKLKQKQANLVLKII | |
| | EQLPSAKESPDKFLEVCTWVDQIAALNDSK | |
| | TRKTTSETVRAVLDSLSEKKKSSP | |
| 4 | ATGAATACAAAATATAATAAAGAGTTCTTA | ATP 5-6x.87 meganuclease nucleic |
| | CTCTACTTAGCAGGGTTTGTAGACGGTGAC | acid sequence |
| | GGTTCCATCACTGCCTGTATCCAGCCTGGG | |
| | CAACATTGTAAGTTCAAGCACGTGCTGAGG | |
| | CTCCGGTTCGGGGTCAGTCAGAAGACACAG | |
| | CGCCGTTGGTTCCTCGACAAGCTGGTGGAC | |
| | GAGATCGGTGTGGGTTACGTGTATGACCTT | |
| | GGCAGCGTCTCCGAGTACCGGCTGTCCCAG | |
| | ATCAAGCCTTTGCATAATTTTTTAACACAA | |
| | CTACAACCTTTTCTAAAACTAAAACAAAAA | |
| | CAAGCAAATTTAGTTTTAAAAATTATTGAA | |
| | CAACTTCCGTCAGCAAAAGAATCCCCGGAC | |
| | AAATTCTTAGAAGTTTGTACATGGGTGGAT | |
| | CAAATTGCAGCTCTGAATGATTCGAAGACG | |
| | CGTAAAACAACTTCTGAAACCGTTCGTGCC | |
| | GTGCTAGACAGTTTACCAGGATCCGTGGGA | |
| | GGTCTATCGCCATCTCAGGCATCCAGCGCC | |
| | GCATCCTCGGCTTCCTCAAGCCCGGGTTCA | |
| | GGGATCTCCGAAGCACTCAGAGCTGGAGCA | |
| | GGTTCCGGCACTGGATACAACAAGGAATTC | |
| | CTGCTCTACCTGGCGGGCTTCGTCGACAGC | |
| | GACGGCTCCATCTTTGCCGCGATCCTTCCT | |
| | CAGCAACCTAGGAAGTTCAAGCACGCTCTG | |
| | CAGCTCTTTTTCGTTGTCACGCAGAAGACA | |
| | CAGCGCCGTTGGTTCCTCGACAAGCTGGTG | |
| | GACGAGATCGGTGTGGGTTACGTGGTGGAC | |
| | CGGGGCAGCGTCTCCGATTACAAGCTGTCC | |
| | CAGATCAAACCTCTGCACAACTTCCTGACC | |
| | CAGCTCCAGCCCTTCCTGAAGCTCAAGCAG | |
| | AAGCAGGCCAACCTCGTGCTGAAGATCATC | |
| | GAGCAGCTGCCCTCCGCCAAGGAATCCCCG | |
| | GACAAGTTCCTGGAGGTGTGCACCTGGGTG | |
| | GACCAGATCGCCGCTCTGAACGACTCCAAG | |
| | ACCCGCAAGACCACTTCCGAAACCGTCCGC | |
| | GCCGTTCTAGACAGTCTCTCCGAGAAGAAG | |
| | AAGTCGTCCCCC | |
| 5 | MNTKYNKEFLLYLAGFVDGDGSICAVIRPS | ATP 7-8x.9 meganuclease amino |
| | QAYKFKHQLQLVFAVAQKTQRRWELDKLVD | acid sequence |
| | EIGVGYVTDNGSVSLYRLSQIKPLHNELTQ | |
| | LQPFLKLKQKQANLVLKIIEQLPSAKESPD | |
| | KFLEVCTWVDQIAALNDSKTRKTTSETVRA | |
| | VLDSLPGSVGGLSPSQASSAASSASSSPGS | |
| | GISEALRAGAGSGTGYNKEFLLYLAGEVDS | |
| | DGSIFAAIQPCQRAKFKHLLHLREDVAQKT | |
| | QRRWFLDKLVDEIGVGYVADNGSVSNYRLS | |
| | QIKPLHNFLTQLQPFLKLKQKQANLVLKII | |
| | EQLPSAKESPDKFLEVCTWVDQIAALNDSK | |
| | TRKTTSETVRAVLDSLSEKKKSSP | |
| 6 | ATGAATACAAAATATAATAAAGAGTTCTTA | ATP 7-8x.9 meganuclease nucleic |
| | CTCTACTTAGCAGGGTTTGTAGACGGTGAC | acid sequence |
| | GGTTCCATCTGTGCAGTGATCCGGCCTTCT | |
| | CAAGCGTATAAGTTCAAGCACCAGCTGCAG | |
| | CTCGTTTTTGCTGTCGCGCAAAAGACACAG | |
| | CGCCGTTGGTTCCTCGACAAGCTGGTGGAC | |
| | GAGATCGGTGTGGGTTACGTGACTGACAAT | |
| | GGCAGCGTCTCCCTGTACCGGCTGTCCCAG | |
| | ATCAAGCCTTTGCATAATTTTTTAACACAA | |
| | CTACAACCTTTTCTAAAACTAAAACAAAAA | |
| | CAAGCAAATTTAGTTTTAAAAATTATTGAA | |
| | CAACTTCCGTCAGCAAAAGAATCCCCGGAC | |
| | AAATTCTTAGAAGTTTGTACATGGGTGGAT | |
| | CAAATTGCAGCTCTGAATGATTCGAAGACG | |
| | CGTAAAACAACTTCTGAAACCGTTCGTGCT | |
| | GTGCTAGACAGTTTGCCAGGATCCGTGGGA | |
| | GGTCTATCGCCATCTCAGGCATCCAGCGCC | |
| | GCATCCTCGGCTTCCTCAAGCCCGGGTTCA | |
| | GGGATCTCCGAAGCACTCAGAGCTGGAGCA | |
| | GGTTCCGGCACTGGATACAACAAGGAATTC | |
| | CTGCTCTACCTGGCGGGCTTCGTCGACTCC | |
| | GACGGCTCCATCTTTGCCGCGATCCAGCCT | |
| | TGTCAACGGGCGAAGTTCAAGCACCTTCTG | |
| | CATCTCCGTTTCGATGTCGCTCAGAAGACA | |
| | CAGCGCCGTTGGTTCCTCGACAAGCTGGTG | |
| | GACGAGATCGGTGTGGGTTACGTGGCGGAC | |
| | AATGGCAGCGTCTCCAATTACAGGCTGTCC | |
| | CAGATCAAGCCTCTGCACAACTTCCTGACC | |
| | CAGCTCCAGCCCTTCCTGAAGCTCAAGCAG | |
| | AAGCAGGCCAACCTCGTGCTGAAGATCATC | |
| | GAGCAGCTGCCCTCCGCCAAGGAATCCCCG | |
| | GACAAGTTCCTGGAGGTGTGCACCTGGGTG | |
| | GACCAGATCGCCGCTCTGAACGACTCCAAG | |
| | ACCCGCAAGACCACTTCCGAAACCGTCCGC | |
| | GCCGTTCTAGACAGTCTCTCCGAGAAGAAG | |
| | AAGTCGTCCCCC | |
| 7 | MASRRLLTSLLRQSAQRGGGPISRSLGNSI | ATPase-ß MTP |
| | PKSAARASSRASPKGELLNRAVQYATSAAA | |
| | PASQPS | |
| 8 | MASRRLLTSLLRQSAQRGGGPISRSLGNSI | ATPase-ß MTP (minus 12 C-terminal |
| | PKSAARASSRASPKGELLNRAVQY | amino acids) |
| 9 | MLSLRQSIRFFKPATRTLCSSRYLLQQKP | COXIV MTP |
| 10 | MGSSFSASFTNSTTAAAVPPPSPPSSPSRS | M20 MTP |
| | NVKSNGEERPRF | |
| 11 | MSVLTPLLLRGLTGSARRLPVPRAKIHSLP | COXVIII-SU9 MTP |
| | PEGKLMASTRVLASRLASQMAASAKVARPA | |
| | VRVAQVSKRTIQTGSPLQTLKRTQMTSIVN | |
| | ATTRQAFQ | |
| 12 | VDEMTKKFGTLTIHDTEK | MVMp NS2 NES sequence |
| 13 | LGAGLGALGL | NES sequence |
| 14 | GATGGGATTGCACGCATTAATAA | ATP 5-6 and ATP 7-8 recognition |
| | | sequences following ligation |
| 15 | ATGGAACTTTCTCCCCGAGCTGCGGAACTA | mtATP1 gene following ligation of |
| | ACAAGTCTATTAGAAAGTCGAATTAGCAAC | ATP 5-6 and ATP 7-8 recognition |
| | TTTTACACCAATTTTCAAGTGGATGAGATC | sequences |
| | GGTCGAGTGGTCTCAGTTGGAGATGGGATT | |
| | GCACGCATTAATAATCTATGATGATCTTAG | |
| | TAAACAGGCGGTAGCATATCGACAAATGTC | |
| | ATTATTGTTACGCCGACCACCAGGTCGTGA | |
| | GGCTTTCCCAGGGGATGTTTTCTATTTACA | |
| | TTCCCGTCTCTTAGAAAGAGCGGCTAAACG | |
| | ATCGGACCAGACAGGCGCAGGTAGCTTGAC | |
| | CGCCTTACCCGTCATTGAAACACAGGCTGG | |
| | AGACGTATCGGCCTATATTCCCACCAATGT | |
| | GATCCCCATTACTGATGGACAAATCTGTTT | |
| | GGAAACAGAGCTCTTTTATCGCGGAATTAG | |
| | ACCTGCGATTAACGTCGGCTTATCTGTCAG | |
| | TCGCGTCGGGTCTGCCGCTCAGTTGAAAAC | |
| | TATGAAACAAGTCTGCGGTAGTTCAAAACT | |
| | GGAATTGGCACAATATCGCGAAGTGGCCGC | |
| | CCTTGCTCAATTTGGCTCAGACCTTGATGC | |
| | TGCGACTCAGGCATTACTCAATAGAGGTGC | |
| | AAGGCTGACAGAAGTACCGAAACAACCACA | |
| | ATATGCACCACTGCCAATTGAAAAACAAAT | |
| | TCTAGTCATTTATGCAGCTGTCAATGGATT | |
| | CTGTGATCGAATGCCACTAGACAGAATTTC | |
| | TCAATATGAGAGAGCCATTCCAAATAGTGT | |
| | CAAACCAGAATTACTACAATCCTTTTTAGA | |
| | AAAAGGTGGCTTAACTAACGAAAGAAAGAT | |
| | GGAACCAGATACATTCTTAAAAGAAAGTGC | |
| | TTTAGCTTTTATTTAA | |
| 16 | CTACCCTAACGTGCACAAATAC | ATP 5-6 recognition sequence |
| | | (antisense) |
| 17 | ATTACCTTACGTGCGTAATTAT | ATP 7-8 recognition sequence |
| | | (antisense) |
| 18 | MNTKYNKEFLLYLAGFVDGDGSIIAQIKPN | Wild-type I-CreI sequence |
| | QSYKFKHQLSLAFQVTQKTQRRWELDKLVD | |
| | EIGVGYVRDRGSVSDYILSEIKPLHNELTQ | |
| | LQPFLKLKQKQANLVLKIIWRLPSAKESPD | |
| | KFLEVCTWVDQ IAALNDSKTRKTTSETVRA | |
| | VLDSLSEKKKSSP | |
| 19 | ATGgaactttctccccgagctgcggaacta | cDNA sequence of transcripts |
| | acaagtctattagaaagtcgaattagcaac | derived from the tobacco mtATP1 |
| | ttttacaccaattttcaagtggatgagatc | gene |
| | ggtcgagtggtctcagttggagatgggatt | |
| | gcacgtgtttatggattgaacgagattcaa | |
| | gctggggaaatggttgaatttgccagcggt | |
| | gtgaaaggaatagccttgaatcttgagaat | |
| | gagaatgtagggattgttgtctttggtagt | |
| | gatactgctattaaagaaggagatcttgtc | |
| | aagcgcactggatctattgtggatgttcct | |
| | gcgggaaaggctatgctagggcgtgtggtc | |
| | gatggcttgggagtacctattgatggaagg | |
| | ggggctctaagcgatcacgagcgaagacgt | |
| | gtcgaagtgaaagcccctggtattattgaa | |
| | cgtaaatctgtgcacgagcctatgcaaaca | |
| | gggttaaaagcggtagatagcctggttcct | |
| | ataggtcgtggtcaacgagaacttataatc | |
| | ggggaccgacaaactggaaaaactgctatt | |
| | gctatcgataccatattaaaccaaaagcaa | |
| | ctgaactcaagggccacctctgagagtgag | |
| | acattgtattgtgtctatgtagcgattgga | |
| | cagaaacgctcaactgtggcacaattagtt | |
| | caaattctttcagaagcgaatgctttggaa | |
| | tattctattcttgtagcagccaccgcttcg | |
| | gatcctgctcctctacaatttttggcccca | |
| | tattctgggtgtgccatgggggaatatttc | |
| | cgcgataatggaatgcacgcattaataatc | |
| | tatgatgatcttagtaaacaggcggtagca | |
| | tatcgacaaatgtcattattgttacgccga | |
| | ccaccaggtcgtgaggctttcccaggggat | |
| | gttttctatttacattcccgtctcttagaa | |
| | agagcggctaaacgatcggaccagacaggc | |
| | gcaggtagcttgaccgccttacccgtcatt | |
| | gaaacacaggctggagacgtatcggcctat | |
| | attcccaccaatgtgatctccattactgat | |
| | ggacaaatctgtttggaaacagagctcttt | |
| | tatcgcggaattagacctgcgattaacgtc | |
| | ggcttatctgtcagtcgcgtcgggtctgcc | |
| | gctcagttgaaaactatgaaacaagtctgc | |
| | ggtagtttaaaactggaattggcacaatat | |
| | cgcgaagtggccgcctttgctcaatttggc | |
| | tcagaccttgatgctgcgactcaggcatta | |
| | ctcaatagaggtgcaaggctgacagaagta | |
| | ctgaaacaaccacaatatgcaccactgcca | |
| | attgaaaaacaaattctagtcatttatgca | |
| | gctgtcaatggattctgtgatcgaatgcca | |
| | ctagacagaatttctcaatatgagagagcc | |
| | attctaaatagtgtcaaaccagaattacta | |
| | caatcctttttagaaaaaggtggcttaact | |
| | aacgaaagaaagatggaactagatacattc | |
| | ttaaaagaaagtgctttagcttttattTAA | |
| 20 | GGATCCACTATGGCTTCTCGGAGGCTTCTC | Sequence of nATP1 |
| | ACCTCTCTCCTCCGTCAATCGGCTCAACGT | |
| | GGCGGCGGTCCAATTTCCCGATCCTTGGGA | |
| | AACTCCATCCCTAAATCCGCTGCACGCGCC | |
| | TCTTCACGCGCGTCCCCTAAGGGATTCCTC | |
| | TTAAACCGCGCCGTACAGTACGCTACCTCT | |
| | GCAGCAGCACCCGCATCTCAGCCATCCatg | |
| | gaactctcaccaagggcagcagaacttact | |
| | tcactcctcgaatctagaatctcaaacttc | |
| | tacacaaactttcaggttgatgaaattgga | |
| | agagttgtttcagttggagatggtattgct | |
| | agggtttatggacttaatgagattcaagct | |
| | ggtgaaatggttgagtttgcttctggagtt | |
| | aagggtatcgctttgaatcttgaaaatgag | |
| | aacgttggaatcgttgtttttggttcagat | |
| | actgctattaaggagggagatttggttaaa | |
| | agaacaggttctattgttgatgttccagct | |
| | ggaaaagctatgcttggtagagttgttgat | |
| | ggattgggtgttcctattgatggaaggggt | |
| | gctttgtctgatcatgaaagaaggagagtt | |
| | gaggttaaggctccaggaattattgaaaga | |
| | aaatcagttcatgagcctatgcaaactggt | |
| | cttaaggctgttgattctttggttccaatt | |
| | ggaaggggtcaaagagaacttattattgga | |
| | gataggcaaactggaaagactgctatcgct | |
| | atcgatacaatccttaaccaaaagcaattg | |
| | aattcaagagctacttctgaatcagagaca | |
| | ttgtattgtgtttacgttgctattggacaa | |
| | aaaaggtctactgttgctcaacttgttcaa | |
| | attttgtctgaagctaatgctcttgagtat | |
| | tcaattttggttgctgctacagcttctgat | |
| | ccagctcctcttcaatttttggctccatat | |
| | tcaggatgcgctatgggtgaatactttaga | |
| | gataatggtatgcatgctcttattatatat | |
| | gatgatttgtcaaagcaagctgttgcttac | |
| | agacaaatgtctcttttgcttagaagacca | |
| | ccaggaagggaagcttttcctggagatgtt | |
| | ttctatcttcattctaggttgcttgagaga | |
| | gctgctaaaaggtctgatcaaactggagct | |
| | ggttcacttacagctttgccagttattgag | |
| | actcaagctggagatgtttcagcttacatc | |
| | cctactaacgttatctctatcacagatgga | |
| | caaatttgtcttgaaacagagttgttttac | |
| | aggggtattagaccagctattaatgttgga | |
| | ctttctgtttcaagagttggttcagctgct | |
| | caattgaagactatgaaacaagtttgcggt | |
| | tctttgaagcttgaattggctcaatatagg | |
| | gaggttgctgcttttgctcaatttggatca | |
| | gatttggatgctgctactcaagctttgctt | |
| | aataggggtgctagacttacagaagttttg | |
| | aagcaacctcaatatgctccacttcctatc | |
| | gagaagcaaattcttgttatatatgctgct | |
| | gttaatggtttctgtgatagaatgccactt | |
| | gataggatctctcaatacgaaagagctatc | |
| | ttgaactcagttaagcctgaattgcttcaa | |
| | tcttttcttgagaaaggaggtttgacaaac | |
| | gagaggaagatggagctcgatacttttctc | |
| | aaggagtccgcactcgcattcatctgaAGA | |
| | TCTATAGATTATAAACTTCTGTGACTTTCT | |
| | TTTCTTCTCTTTGCCAAAATAATTTAGTTT | |
| | GTGACATCCCGGATTTTTTTGGAGGACCAA | |
| | GAGGTCCAGAATTCTGGTTTTGTTTTACAT | |
| | CCAATGCGAGATTATAGAGACATGCAGCCA | |
| | AGCCTTTGTTGCCAGAGACCCCCTTTTCTG | |
| | TTATGTCACATAATAAAGGGGGTAAATGGT | |
| | GATCTTGTATATCTGATTTTCAAGTCTTTT | |
| | TCGAGAATTTTGGATTCCCTGATTATCAAA | |
| | TGCCTTTCTGAAACGCTTTCTTTCTATATG | |
| | TGGTTAACTTCACGTCCATTTTATCCATTC | |
| | TGCCTTGAAAGCTTCAATGTAATAGGAGCA | |
| | GTTAATATGCTAAGCGGATACAAATCATAT | |
| | TTCTTGGCGCGACTTAATTTTATTGCAGTA | |
| | TGAATACTTGTAGAAAATATGAGTATTTGA | |
| | CTAAGCTTATAAGCGGCAAACCAGCTTATA | |
| | AGTCACTTTTACTTTACTTCATCTACGCGT | |
| | TTGGTAAAATTAAAAGTGTTAAGTCTAGTG | |
| | CTTGTAAGCTTTTAAGTCTTAAGTTGTCAT | |
| | AAGTTGGTCACATCTAAATTTGAACTGCCC | |
| | CTGCCCCCATCTTCCCCAAAAGAATCTGCA | |
| | TTCACGCTAAGAGGTTATGCAAACTCTTTA | |
| | GGGCTAAAGAATTGTCGTCAAATTATAACT | |
| | AAAGCCAAAGTTCTAAAATATGTCTTACAG | |
| | AGATAATGTCTAGAGAATAATAAGATTTAT | |
| | ATGCAGAGACAATTCCTTTAGATTCCTTTA | |
| | GGATGATAATAACACACGCCAGACATACAT | |
| | TATAGTGGAAAAAATAATGAACAGGAAATT | |
| | ACACCCAATAACCTTGAACATATATGGTCT | |
| | TGATTTTTTGATTCTTTGTCCCGGTACC | |
| 21 | GTCGACGAGAGGCGGTTTGCGTATTGGCTA | Sequence of construct 35S:nATP1 |
| | GAGCAGCTTGCCAACATGGTGGAGCACGAC | |
| | ACTCTCGTCTACTCCAAGAATATCAAAGAT | |
| | ACAGTCTCAGAAGACCAAAGGGCTATTGAG | |
| | ACTTTTCAACAAAGGGTAATATCGGGAAAC | |
| | CTCCTCGGATTCCATTGCCCAGCTATCTGT | |
| | CACTTCATCAAAAGGACAGTAGAAAAGGAA | |
| | GGTGGCACCTACAAATGCCATCATTGCGAT | |
| | AAAGGAAAGGCTATCGTTCAAGATGCCTCT | |
| | GCCGACAGTGGTCCCAAAGATGGACCCCCA | |
| | CCCACGAGGAGCATCGTGGAAAAAGAAGAC | |
| | GTTCCAACCACGTCTTCAAAGCAAGTGGAT | |
| | TGATGTGAACATGGTGGAGCACGACACTCT | |
| | CGTCTACTCCAAGAATATCAAAGATACAGT | |
| | CTCAGAAGACCAAAGGGCTATTGAGACTTT | |
| | TCAACAAAGGGTAATATCGGGAAACCTCCT | |
| | CGGATTCCATTGCCCAGCTATCTGTCACTT | |
| | CATCAAAAGGACAGTAGAAAAGGAAGGTGG | |
| | CACCTACAAATGCCATCATTGCGATAAAGG | |
| | AAAGGCTATCGTTCAAGATGCCTCTGCCGA | |
| | CAGTGGTCCCAAAGATGGACCCCCACCCAC | |
| | GAGGAGCATCGTGGAAAAAGAAGACGTTCC | |
| | AACCACGTCTTCAAAGCAAGTGGATTGATG | |
| | TGATATCTCCACTGACGTAAGGGATGACGC | |
| | ACAATCCCACTATCCTTCGCAAGACCCTTC | |
| | CTCTATATAAGGAAGTTCATTTCATTTGGA | |
| | GAGGACACGCTGAAATCACCAGTCTCTCTC | |
| | TACAAATCTATCTCTCTCGAGCTTTCGCAG | |
| | ATCTGTCGAACCAGGATCCACTATGGCTTC | |
| | TCGGAGGCTTCTCACCTCTCTCCTCCGTCA | |
| | ATCGGCTCAACGTGGCGGCGGTCCAATTTC | |
| | CCGATCCTTGGGAAACTCCATCCCTAAATC | |
| | CGCTGCACGCGCCTCTTCACGCGCGTCCCC | |
| | TAAGGGATTCCTCTTAAACCGCGCCGTACA | |
| | GTACGCTACCTCTGCAGCAGCACCCGCATC | |
| | TCAGCCATCCatggaactctcaccaagggc | |
| | agcagaacttacttcactcctcgaatctag | |
| | aatctcaaacttctacacaaactttcaggt | |
| | tgatgaaattggaagagttgtttcagttgg | |
| | agatggtattgctagggtttatggacttaa | |
| | tgagattcaagctggtgaaatggttgagtt | |
| | tgcttctggagttaagggtatcgctttgaa | |
| | tcttgaaaatgagaacgttggaatcgttgt | |
| | ttttggttcagatactgctattaaggaggg | |
| | agatttggttaaaagaacaggttctattgt | |
| | tgatgttccagctggaaaagctatgcttgg | |
| | tagagttgttgatggattgggtgttcctat | |
| | tgatggaaggggtgctttgtctgatcatga | |
| | aagaaggagagttgaggttaaggctccagg | |
| | aattattgaaagaaaatcagttcatgagcc | |
| | tatgcaaactggtcttaaggctgttgattc | |
| | tttggttccaattggaaggggtcaaagaga | |
| | acttattattggagataggcaaactggaaa | |
| | gactgctatcgctatcgatacaatccttaa | |
| | ccaaaagcaattgaattcaagagctacttc | |
| | tgaatcagagacattgtattgtgtttacgt | |
| | tgctattggacaaaaaaggtctactgttgc | |
| | tcaacttgttcaaattttgtctgaagctaa | |
| | tgctcttgagtattcaattttggttgctgc | |
| | tacagcttctgatccagctcctcttcaatt | |
| | tttggctccatattcaggatgcgctatggg | |
| | tgaatactttagagataatggtatgcatgc | |
| | tcttattatatatgatgatttgtcaaagca | |
| | agctgttgcttacagacaaatgtctctttt | |
| | gcttagaagaccaccaggaagggaagcttt | |
| | tcctggagatgttttctatcttcattctag | |
| | gttgcttgagagagctgctaaaaggtctga | |
| | tcaaactggagctggttcacttacagcttt | |
| | gccagttattgagactcaagctggagatgt | |
| | ttcagcttacatccctactaacgttatctc | |
| | tatcacagatggacaaatttgtcttgaaac | |
| | agagttgttttacaggggtattagaccagc | |
| | tattaatgttggactttctgtttcaagagt | |
| | tggttcagctgctcaattgaagactatgaa | |
| | acaagtttgcggttctttgaagcttgaatt | |
| | ggctcaatatagggaggttgctgcttttgc | |
| | tcaatttggatcagatttggatgctgctac | |
| | tcaagctttgcttaataggggtgctagact | |
| | tacagaagttttgaagcaacctcaatatgc | |
| | tccacttcctatcgagaagcaaattcttgt | |
| | tatatatgctgctgttaatggtttctgtga | |
| | tagaatgccacttgataggatctctcaata | |
| | cgaaagagctatcttgaactcagttaagcc | |
| | tgaattgcttcaatcttttcttgagaaagg | |
| | aggtttgacaaacgagaggaagatggagct | |
| | cgatacttttctcaaggagtccgcactcgc | |
| | attcatctgaAGATCTATAGATTATAAACT | |
| | TCTGTGACTTTCTTTTCTTCTCTTTGCCAA | |
| | AATAATTTAGTTTGTGACATCCCGGATTTT | |
| | TTTGGAGGACCAAGAGGTCCAGAATTCTGG | |
| | TTTTGTTTTACATCCAATGCGAGATTATAG | |
| | AGACATGCAGCCAAGCCTTTGTTGCCAGAG | |
| | ACCCCCTTTTCTGTTATGTCACATAATAAA | |
| | GGGGGTAAATGGTGATCTTGTATATCTGAT | |
| | TTTCAAGTCTTTTTCGAGAATTTTGGATTC | |
| | CCTGATTATCAAATGCCTTTCTGAAACGCT | |
| | TTCTTTCTATATGTGGTTAACTTCACGTCC | |
| | ATTTTATCCATTCTGCCTTGAAAGCTTCAA | |
| | TGTAATAGGAGCAGTTAATATGCTAAGCGG | |
| | ATACAAATCATATTTCTTGGCGCGACTTAA | |
| | TTTTATTGCAGTATGAATACTTGTAGAAAA | |
| | TATGAGTATTTGACTAAGCTTATAAGCGGC | |
| | AAACCAGCTTATAAGTCACTTTTACTTTAC | |
| | TTCATCTACGCGTTTGGTAAAATTAAAAGT | |
| | GTTAAGTCTAGTGCTTGTAAGCTTTTAAGT | |
| | CTTAAGTTGTCATAAGTTGGTCACATCTAA | |
| | ATTTGAACTGCCCCTGCCCCCATCTTCCCC | |
| | AAAAGAATCTGCATTCACGCTAAGAGGTTA | |
| | TGCAAACTCTTTAGGGCTAAAGAATTGTCG | |
| | TCAAATTATAACTAAAGCCAAAGTTCTAAA | |
| | ATATGTCTTACAGAGATAATGTCTAGAGAA | |
| | TAATAAGATTTATATGCAGAGACAATTCCT | |
| | TTAGATTCCTTTAGGATGATAATAACACAC | |
| | GCCAGACATACATTATAGTGGAAAAAATAA | |
| | TGAACAGGAAATTACACCCAATAACCTTGA | |
| | ACATATATGGTCTTGATTTTTTGATTCTTT | |
| | GTCCCGGTACC | |
| 22 | GTCGACATAGACCCCCAAAGGGATTGTTCA | Sequence of construct βprom:nATP1 |
| | AACTAAACATAGATGCAAGTTTCAACAAAA | |
| | ATTTGCAAAATTGTGCCCTGGGTGGAGTAA | |
| | TCAGAAATTAATGCTAATGGTCACTGGATA | |
| | GTTGGCTTTGCAAAATCAGCTCATGCACGT | |
| | GGGTCACTACATGCTGAGATCAAAACATTA | |
| | CTAGCAGGACTTAAAACAACTCATACTTGG | |
| | GGGATGTTCCCTCTCCAAATTGAACAATAT | |
| | TAATTATTCTTCTTAATTTGTACCAAGTTA | |
| | TATTACGTATCCTTAAAATCGCATATAAAT | |
| | TCAACTGTTATCCGATTTTAGGGTAAACAG | |
| | TTTGGCGATCACCGTGGGGCTAAGGATAAT | |
| | GGTGATTACCTGGTACAAACTTTCATGACA | |
| | CACACTATTTTACACTTGTTCTTTGAAGTG | |
| | TCTTTGATTACAGGACTAAAAAATATCAAA | |
| | CTCTCAGTCAGCACCTCTACACATTGACAA | |
| | TGATTTCGGCCGCCACGACGAAAATGATAA | |
| | CATAGCAGCAGGGAACGATGTACCAGTATT | |
| | AGCCTCGATGAATTTTCGGCCGCGGATCTA | |
| | GTGGACGTCAGTTCACATATAACCATCAAC | |
| | GGGAATTTAGCCGTCGATCCCGAAGGCACC | |
| | ATCTGTGGGGATGCTCGATCAGCCGCTCAA | |
| | AGCGCATCTGGCGGTGAAGATGGTAGGATA | |
| | AGCCTGTAGGTGATCTTCGAAATGTTGCAG | |
| | GCTCAGCAGGCAGCAATAGCCCAGCTCCAA | |
| | AACCAGAATCACACACCAAGCAGGGTTGAG | |
| | CTCGAGCCATCCCAGGAAGTTGTACACATG | |
| | GTCGAATCGGTTTCAAGAAAATCGAGCAAG | |
| | AAAGAGTCGAAGACCAACCCATCAATCATG | |
| | AAGATGCTCGAAGAACTAACGAAGCGAATT | |
| | GAGTCGGGGAAAAAAAAGATCGAGGCAAAC | |
| | GACAAGAAGGTGGAAACTTGTAACTCCAGG | |
| | GTCGATCAAATCCCGGGGGCACCACCAATA | |
| | TTGAAAGGACTAGATTCCAAAAAGTTCATA | |
| | CAAAAACCTTCCCCCCTCCCCCGAGCACGG | |
| | CTGCGAAGCCAATCCCCAAGAAGTTCCGCA | |
| | TGCCCGAGATTCCTAAGTATAATGGAATGA | |
| | CTGATCCGAATGAGCACGTCACCTCTTACA | |
| | CGTGTGCTATCAAAGGGAATGATCTAGAGG | |
| | ATGACGAGATCGAATCCTGTCAATCCAATA | |
| | GGGTAATAATCTTTATGCTAACATTGTCAA | |
| | CAAATGCAGGTTATTAATGCACCAACAAAA | |
| | TGAGGTAATCATCCAGCACGAGTTCAGGCA | |
| | AGGGAATAAGGTAGCTCACCAAATGGCAAA | |
| | GAAGGCAACTGCTGATCTCAGGAAGGAGAA | |
| | AGTTTTTGTGGAACCACCAGATATTGTAAA | |
| | AAATCTAGTAGAAAGTGATTTACCAGAACA | |
| | ATATATTTTTGGAAAACTACTATCTTATGA | |
| | TACTTGTAAAAATTTAGCAAGCCTAGGTAA | |
| | CCAAAGTGTCCTATGCGACACTAACTATGA | |
| | ATCTCATGTACTGCATGTTCCTTAACCTTT | |
| | AATATATATATTTCCTGTTTGCTCAAAAAA | |
| | AAAAATATTAGTTACTTGTAGTTAGTCTTA | |
| | AAACAATTAAATAGTGTAATTTTTGTTTAT | |
| | AATATCGGTTTATAAAAAATAAACCTTAAT | |
| | GAAATTTGCGCATATTTGAGTCAAGTTTAT | |
| | GACCTTTTTGCGTGATATACAACCTTACAT | |
| | TACCATCCAACTATGTTATATGAGTTTTTT | |
| | ATTTATTAGAGATTGTACGTATCAAGTGCA | |
| | AAGAATTTTTTTACTCGTATTTGTTATTTG | |
| | CATCAAATTAATAAATACAAAATACTATGT | |
| | TTTGATTCTTGCACAAATTTTTTATTTTAA | |
| | TTATGATTAAACTATATGTTCTCGTACTAT | |
| | TTTAATTTCATAGATTAATCTATAACAGTA | |
| | TAACTATTGAGTCTCACTTTGTGCAAGTGT | |
| | GGAGCACATATTTGAAAACATAAGGATGCC | |
| | CAAGTTATTCGCCAAAAAAGTTGGAATAAA | |
| | AGCGAAAAAGGAAAACAAAGAAAAAAAAGA | |
| | ACAAATTCAAATGCTCCTCGGTTTTTTAAG | |
| | CACAGTAAAGCAGCAATCGGATCACACAGT | |
| | CGCACAGTGGGCTCTTGATAAATCAGCTCT | |
| | TCATATTTCCCACAAACCCTAGCAGTCTCT | |
| | TTCTCTCTCTACTCCTTTCATCCTCTCTCT | |
| | AACCAAACCCTCCGGATCCACTATGGCTTC | |
| | TCGGAGGCTTCTCACCTCTCTCCTCCGTCA | |
| | ATCGGCTCAACGTGGCGGCGGTCCAATTTC | |
| | CCGATCCTTGGGAAACTCCATCCCTAAATC | |
| | CGCTGCACGCGCCTCTTCACGCGCGTCCCC | |
| | TAAGGGATTCCTCTTAAACCGCGCCGTACA | |
| | GTACGCTACCTCTGCAGCAGCACCCGCATC | |
| | TCAGCCATCCatggaactctcaccaagggc | |
| | agcagaacttacttcactcctcgaatctag | |
| | aatctcaaacttctacacaaactttcaggt | |
| | tgatgaaattggaagagttgtttcagttgg | |
| | agatggtattgctagggtttatggacttaa | |
| | tgagattcaagctggtgaaatggttgagtt | |
| | tgcttctggagttaagggtatcgctttgaa | |
| | tcttgaaaatgagaacgttggaatcgttgt | |
| | ttttggttcagatactgctattaaggaggg | |
| | agatttggttaaaagaacaggttctattgt | |
| | tgatgttccagctggaaaagctatgcttgg | |
| | tagagttgttgatggattgggtgttcctat | |
| | tgatggaaggggtgctttgtctgatcatga | |
| | aagaaggagagttgaggttaaggctccagg | |
| | aattattgaaagaaaatcagttcatgagcc | |
| | tatgcaaactggtcttaaggctgttgattc | |
| | tttggttccaattggaaggggtcaaagaga | |
| | acttattattggagataggcaaactggaaa | |
| | gactgctatcgctatcgatacaatccttaa | |
| | ccaaaagcaattgaattcaagagctacttc | |
| | tgaatcagagacattgtattgtgtttacgt | |
| | tgctattggacaaaaaaggtctactgttgc | |
| | tcaacttgttcaaattttgtctgaagctaa | |
| | tgctcttgagtattcaattttggttgctgc | |
| | tacagcttctgatccagctcctcttcaatt | |
| | tttggctccatattcaggatgcgctatggg | |
| | tgaatactttagagataatggtatgcatgc | |
| | tcttattatatatgatgatttgtcaaagca | |
| | agctgttgcttacagacaaatgtctctttt | |
| | gcttagaagaccaccaggaagggaagcttt | |
| | tcctggagatgttttctatcttcattctag | |
| | gttgcttgagagagctgctaaaaggtctga | |
| | tcaaactggagctggttcacttacagcttt | |
| | gccagttattgagactcaagctggagatgt | |
| | ttcagcttacatccctactaacgttatctc | |
| | tatcacagatggacaaatttgtcttgaaac | |
| | agagttgttttacaggggtattagaccagc | |
| | tattaatgttggactttctgtttcaagagt | |
| | tggttcagctgctcaattgaagactatgaa | |
| | acaagtttgcggttctttgaagcttgaatt | |
| | ggctcaatatagggaggttgctgcttttgc | |
| | tcaatttggatcagatttggatgctgctac | |
| | tcaagctttgcttaataggggtgctagact | |
| | tacagaagttttgaagcaacctcaatatgc | |
| | tccacttcctatcgagaagcaaattcttgt | |
| | tatatatgctgctgttaatggtttctgtga | |
| | tagaatgccacttgataggatctctcaata | |
| | cgaaagagctatcttgaactcagttaagcc | |
| | tgaattgcttcaatcttttcttgagaaagg | |
| | aggtttgacaaacgagaggaagatggagct | |
| | cgatacttttctcaaggagtccgcactcgc | |
| | attcatctgaAGATCTATAGATTATAAACT | |
| | TCTGTGACTTTCTTTTCTTCTCTTTGCCAA | |
| | AATAATTTAGTTTGTGACATCCCGGATTTT | |
| | TTTGGAGGACCAAGAGGTCCAGAATTCTGG | |
| | TTTTGTTTTACATCCAATGCGAGATTATAG | |
| | AGACATGCAGCCAAGCCTTTGTTGCCAGAG | |
| | ACCCCCTTTTCTGTTATGTCACATAATAAA | |
| | GGGGGTAAATGGTGATCTTGTATATCTGAT | |
| | TTTCAAGTCTTTTTCGAGAATTTTGGATTC | |
| | CCTGATTATCAAATGCCTTTCTGAAACGCT | |
| | TTCTTTCTATATGTGGTTAACTTCACGTCC | |
| | ATTTTATCCATTCTGCCTTGAAAGCTTCAA | |
| | TGTAATAGGAGCAGTTAATATGCTAAGCGG | |
| | ATACAAATCATATTTCTTGGCGCGACTTAA | |
| | TTTTATTGCAGTATGAATACTTGTAGAAAA | |
| | TATGAGTATTTGACTAAGCTTATAAGCGGC | |
| | AAACCAGCTTATAAGTCACTTTTACTTTAC | |
| | TTCATCTACGCGTTTGGTAAAATTAAAAGT | |
| | GTTAAGTCTAGTGCTTGTAAGCTTTTAAGT | |
| | CTTAAGTTGTCATAAGTTGGTCACATCTAA | |
| | ATTTGAACTGCCCCTGCCCCCATCTTCCCC | |
| | AAAAGAATCTGCATTCACGCTAAGAGGTTA | |
| | TGCAAACTCTTTAGGGCTAAAGAATTGTCG | |
| | TCAAATTATAACTAAAGCCAAAGTTCTAAA | |
| | ATATGTCTTACAGAGATAATGTCTAGAGAA | |
| | TAATAAGATTTATATGCAGAGACAATTCCT | |
| | TTAGATTCCTTTAGGATGATAATAACACAC | |
| | GCCAGACATACATTATAGTGGAAAAAATAA | |
| | TGAACAGGAAATTACACCCAATAACCTTGA | |
| | ACATATATGGTCTTGATTTTTTGATTCTTT | |
| | GTCCCGGTACC | |
| 23 | GGATCCACTATGGCTTCTCGGAG | nATP1-specific forward primer |
| 24 | CTATAGATCTTCAGATGAATGCG | nATP1-specific reverse primer |
| 25 | AGCACCCTGTTCTTCTCAC | tobacco actin gene control forward |
| | | primer |
| 26 | GTCAAGCTCCTGCTCGTAG | tobacco actin gene control reverse |
| | | primer |
| 27 | CAAGTGGATGAGATCGGTCG | wild-type mtATP1 gene forward |
| | | primer flaking the ATP 5-6 and ATP |
| | | 7-8 recognition sites |
| 28 | ACTGACAGATAAGCCGACGT | wild-type mtATP1 gene reverse |
| | | primer flaking the ATP 5-6 and ATP |
| | | 7-8 recognition sites |
| 29 | ATCCGGGCCTTAATCCTTGC | mtATP6 control forward primer |
| 30 | TGCGAGGGGAAAACTTTTGT | mtATP6 control reverse primer |
| 31 | MNTKYNKEFLLYLAGFVDGDGSIIAQIKPN | ARCUS meganuclease with wild-type |
| | QSYKFKHQLSLAFQVTQKTQRRWELDKLVD | I-CreI subunits |
| | EIGVGYVRDRGSVSDYILSEIKPLHNELTQ | |
| | LQPFLKLKQKQANLVLKIIEQLPSAKESPD | |
| | KFLEVCTWVDQIAALNDSKTRKTTSETVRA | |
| | VLDSLPGSVGGLSPSQASSAASSASSSPGS | |
| | GISEALRAGAGSGTGYNKEFLLYLAGFVDG | |
| | DGSIIAQIKPNQSYKFKHQLSLAFQVTQKT | |
| | QRRWFLDKLVDEIGVGYVRDRGSVSDYILS | |
| | EIKPLHNFLTQLQPFLKLKQKQANLVLKII | |
| | EQLPSAKESPDKFLEVCTWVDQIAALNDSK | |
| | TRKTTSETVRAVLDSLSEKKKSSP | |

### EXAMPLES

This disclosure is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1. ATP 5-6 and ATP 7-8 Nucleases for Mitochondrial Editing in Plants

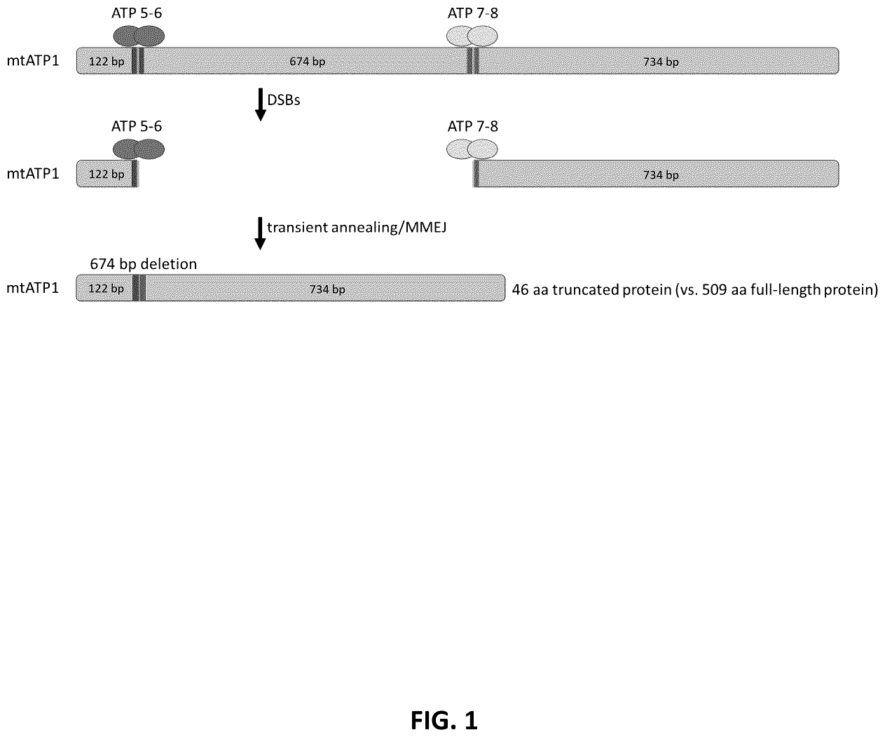

Engineered meganucleases were evaluated using the CHO cell reporter assay previously described (see, WO/2012/167192) to determine whether the ATP 5-6 and ATP 7-8 meganucleases could bind and cleave their respective recognition sequences (i.e., ATP 5-6 and ATP 7-8) in cells. See, FIG. 1.

In the CHO reporter cell lines developed for this study, two recognition sequences were inserted into the GFP gene. One recognition sequence was for the ATP 5-6 or the ATP 7-8 recognition sequence. The second recognition sequence inserted was a CHO-23/24 recognition sequence, which is recognized and cleaved by a control meganuclease referred to herein as CHO-23/24. The CHO-23/24 recognition sequence was used as a positive control and standard measure of activity. To determine whether ATP 5-6 and ATP 7-8 meganucleases could recognize and cleave the ATP 5-6 and ATP 7-8 recognition sequences, each meganuclease was evaluated using the CHO cell reporter assay. To perform the assay, a pair of CHO cell reporter lines was produced which carried a non-functional Green Fluorescent Protein (GFP) gene expression cassette integrated into the genome of the cell. The GFP gene in each cell line was interrupted by a pair of recognition sequences such that intracellular cleavage of either recognition sequence by a meganuclease would stimulate a homologous recombination event resulting in a functional GFP gene. In both cell lines, one of the recognition sequences was derived from the ATP gene and the second recognition sequence was specifically recognized by CHO-23/24. CHO reporter cells comprising the ATP recognition sequences (SEQ ID NO: 1 or SEQ ID NO: 2) and the CHO-23/24 recognition sequence are referred to herein as ATP 5-6 and ATP 7-8 cells.

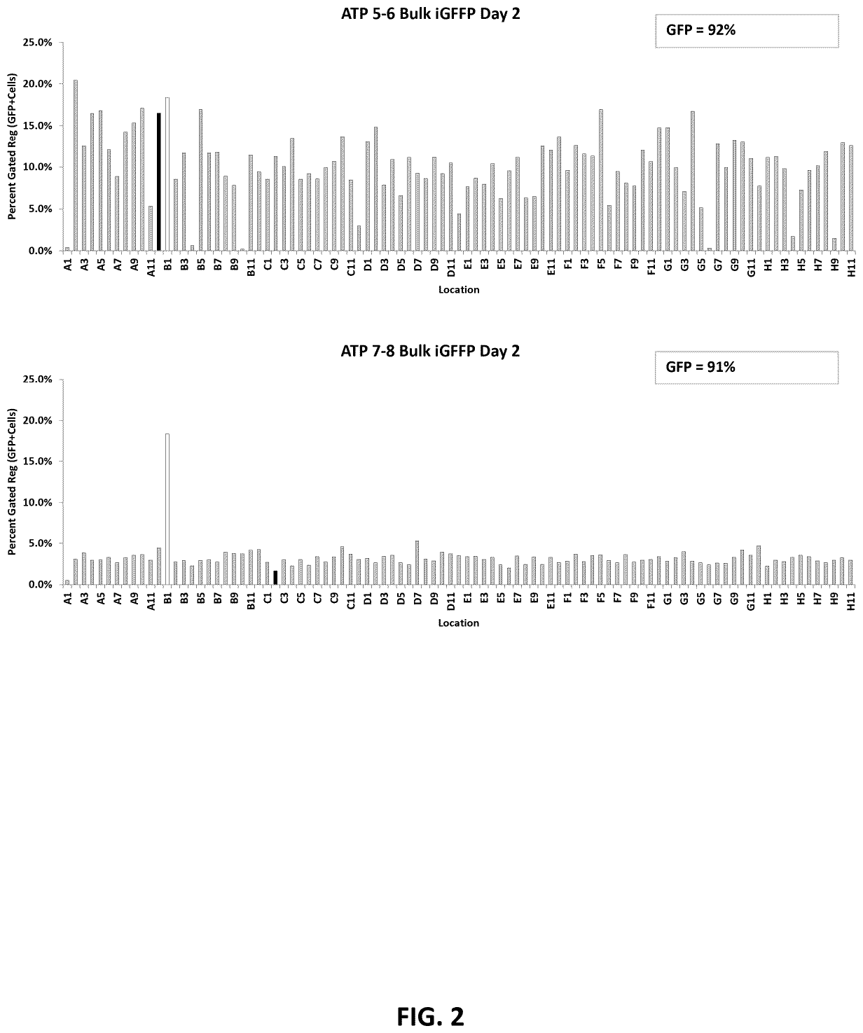

ATP 5-6 and ATP 7-8 cells were transfected with plasmid DNA encoding ATP 5-6 and ATP 7-8 meganuclease variants or encoding the CHO-23/34 meganuclease. 4e5 CHO cells were transfected with 50 ng of plasmid DNA in a 96-well plate using LIPOFECTAMINE 2000 (ThermoFisher) according to the manufacturer's instructions. At 48 hours post-transfection, cells were evaluated by flow cytometry to determine the percentage of GFP-positive cells compared to a non-transfected negative control (1-2 bs). Meganucleases were found to produce GFP-positive cells in cell lines comprising the ATP 5-6 and ATP 7-8 recognition sequence at frequencies significantly exceeding the negative control and comparable to or exceeding the CHO-23/24 positive control, indicating that engineered ATP 5-6 and ATP 7-8 meganucleases of the invention could efficiently and selectively bind and cleave their respective recognition sequences (i.e., ATP 5-6 and ATP 7-8) in cells (FIG. 2).

### Example 2: Mitochondrial Targeting of Nucleases

Five candidate transit peptides were identified and cloned to the N-terminal of a homing nuclease fused to green fluorescent protein (GFP). These transit peptides include three plant-specific transit peptides described in the literature (two variations of the transit peptide from ATPase (3-subunit and one from COXIV), one transit peptide for targeting to the mitochondria in mammalian cells (COXVII-SU9), and a novel transit peptide identified from a native mitochondrially-targeted endonuclease in plants (M20). These TP-MTEM-GFP constructs were transiently delivered to tobacco protoplasts alongside control constructs with MTEM-GFP with a nuclear localization signal or absent any transit peptide/signal. Microscope images were taken 24 h post-transfection to identify localization patterns of the fluorescent MTEM-GFP fusion proteins.

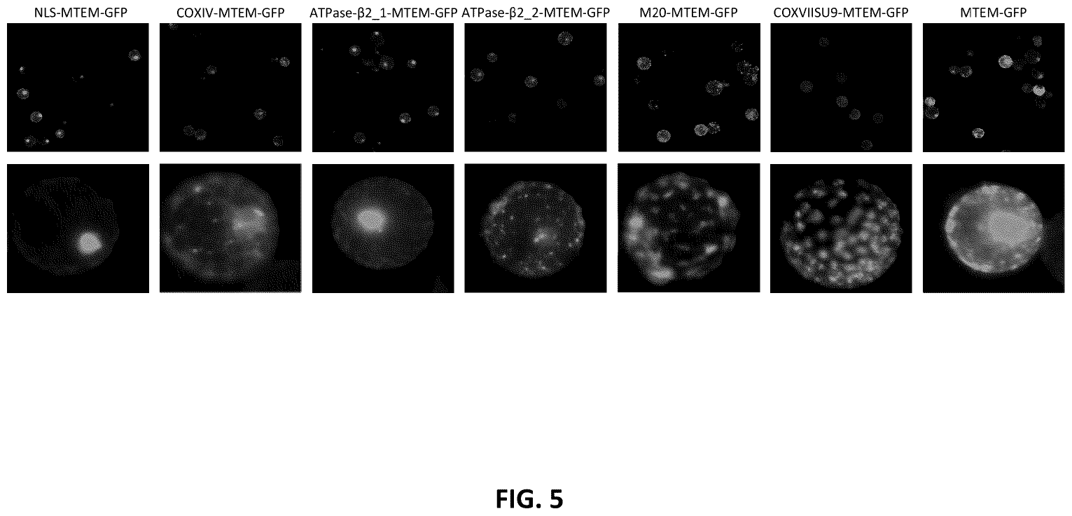

Mitochondrial targeting was most successful in constructs containing M20 (i.e., SEQ ID NO: 10) and several ATPase β-2 transit peptides. M20 and ATPase β-2 transit peptides efficiently directed MTEM-GFP protein to the mitochondria in tobacco cells (FIG. 5).

The ability of a pair of ATP 5-6 and ATP 7-8 nucleases to generate edits in the mitochondrial genome was then tested in a transient protoplast system. Vectors containing the two nucleases driven by constitutive promoters and containing N-terminal mitochondrial transit peptides were transfected into tobacco protoplasts. Samples were collected 72 h after delivery. DNA was extracted and primers were used to amplify a 917 base pair region surrounding the cut sites of ATP 5-6 and ATP 7-8. If the predicted 674 base pair deletion between the cut sites was generated, the annealed product would be 243 base pairs in length. PCR products were purified and sequenced on MiniSeq. Given that this technology constrains paired sequencing reads to approximately 300 base pairs in length, only the deletion product would be sequenced using these primer pairs and the wild-type sequence would be too long to be sequenced. Additional primer pairs targeting shorter regions surrounding the individual cut sites were included as controls.

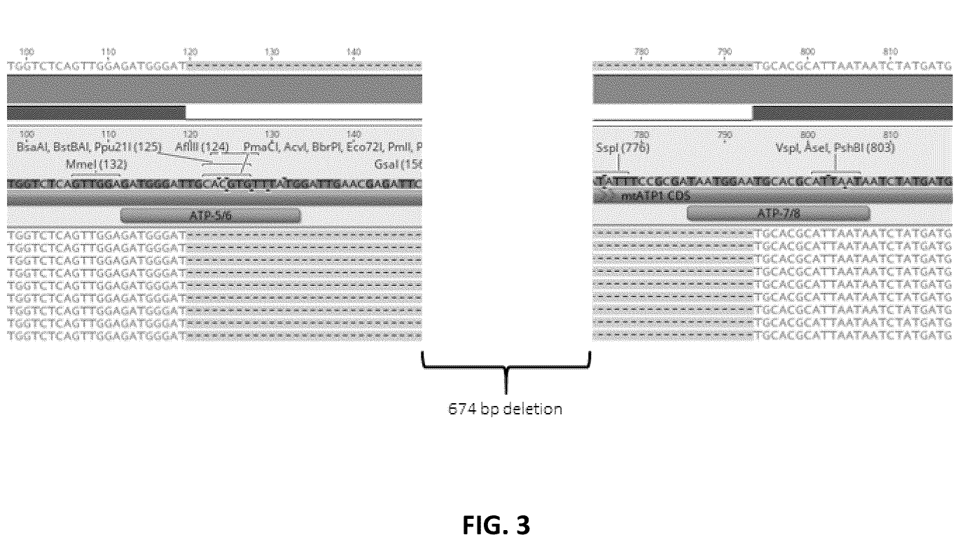

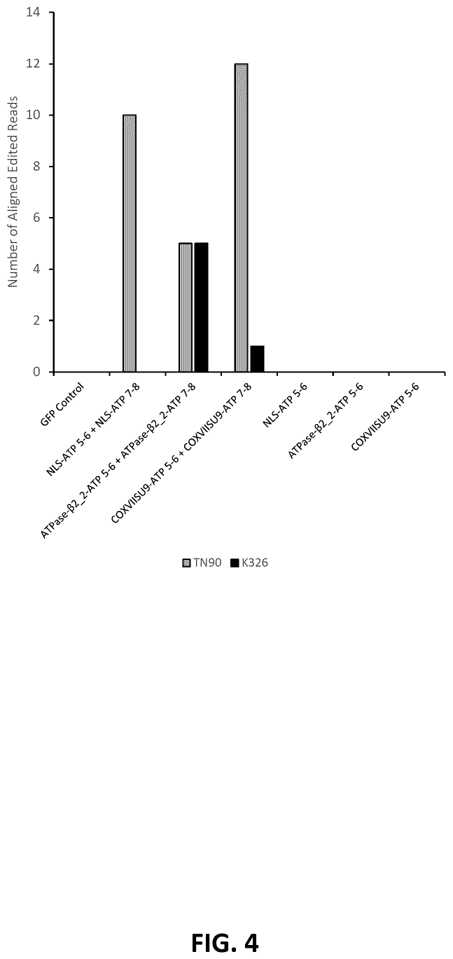

The exact predicted deletion product (spanning 674 bp between ATP 5-6 and ATP 7-8 cut sites) was identified in a small number of reads corresponding to protoplast samples with vectors containing dual ATP 5-6 and ATP 7-8 nucleases (FIG. 3). Though low in frequency, these deletion product reads only occurred in samples with both nucleases delivered and not in control samples lacking either nucleases or with only ATP 5-6 delivered (FIG. 4).

### Example 3: Vectors Generated for CMS System

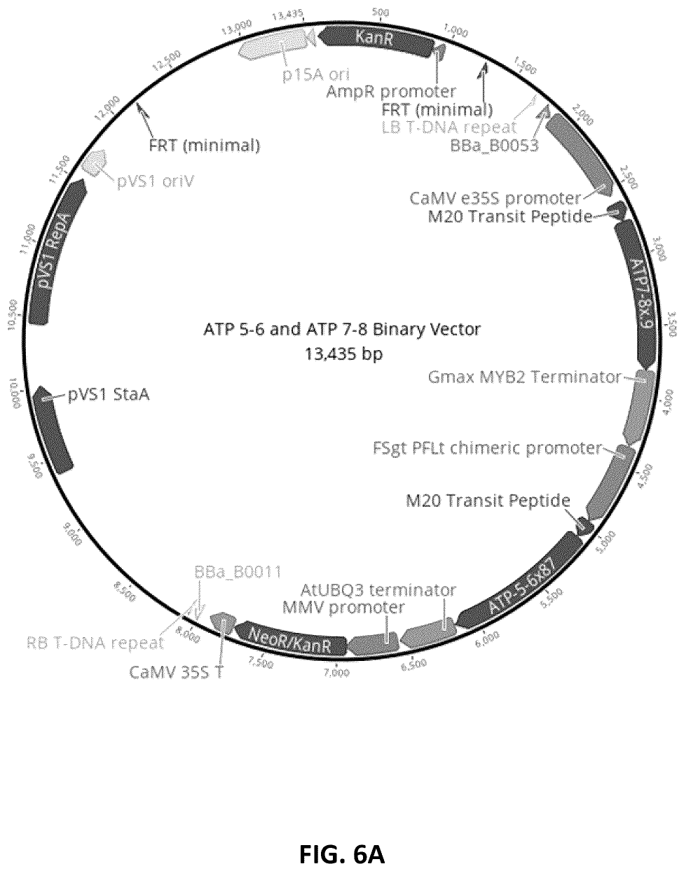

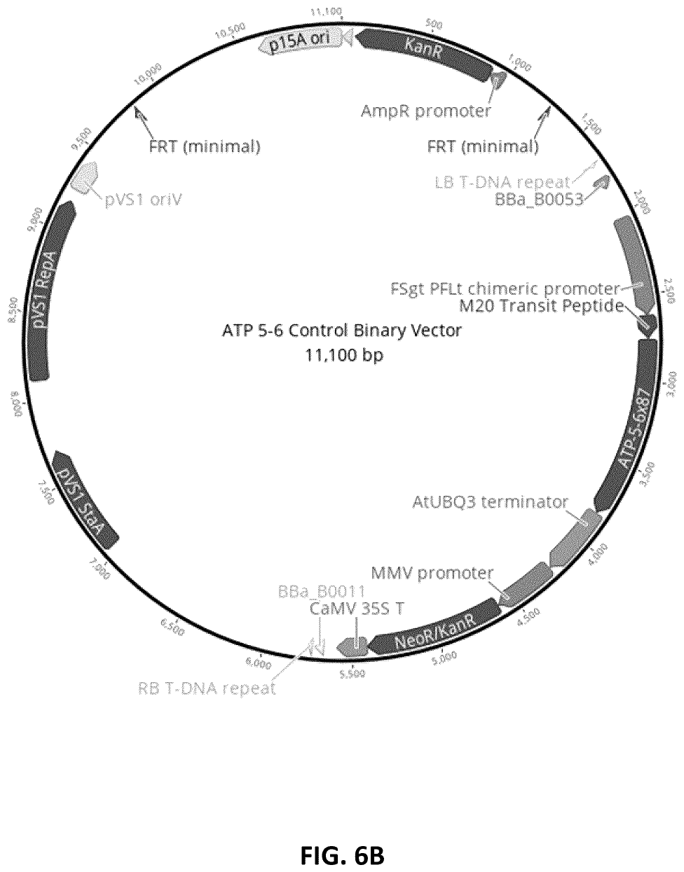

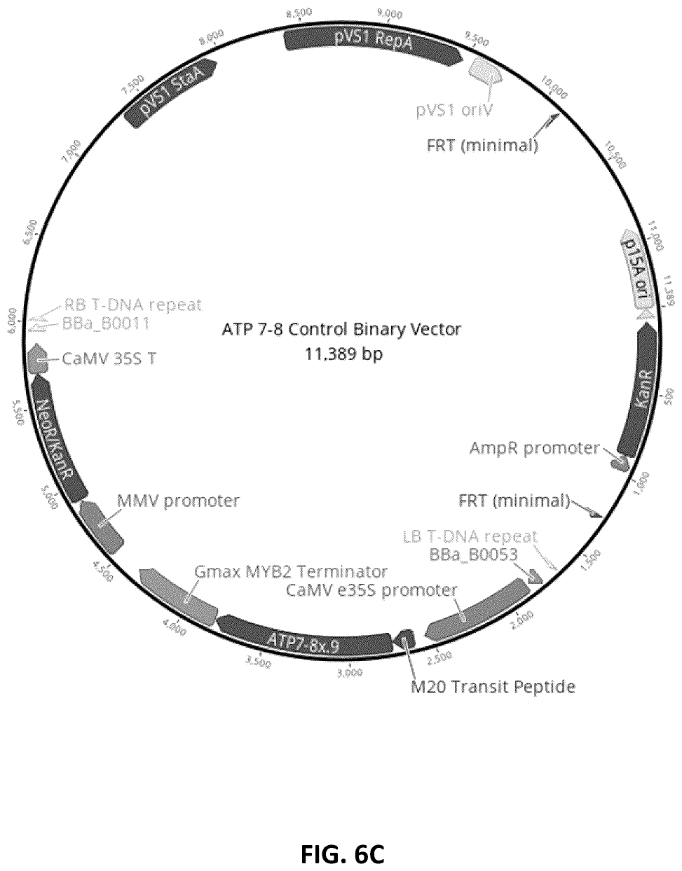

A binary vector containing ATP 5-6 and ATP 7-8 nucleases with N-terminal M20 transit peptides and driven by constitutive promoters was generated alongside control vectors containing ATP 5-6 or ATP 7-8 nucleases alone (FIG. 6A-6C). These vectors are stably transformed in tobacco plants to confirm the deletion product generated in transient delivery of the same combination of nucleases (as described in the examples below).

### Example 4: Transferal of mtATP1 Gene Function to the Nucleus and Generation of CMS Lines

A. Background

In plants, the failure to produce viable pollen is referred to as male sterility. Male sterility is a trait of particular interest to the hybrid seed industry, as it can assist in the controlled pollination of two distinct inbred lines to produce uniform hybrid seed. Mutations in genes encoded by the nuclear genome that inhibit pollen production give rise to genic male sterility (GMS). The genes responsible for GMS segregate according to the standard rules of Mendelian inheritance, and because naturally occurring GMS-conferring mutations are recessive, establishing pure inbred lines that are sterile for the purpose of hybrid seed production is problematic. This is due to the fact that gins/gins homozygous individuals cannot be self-fertilized and in order to be propagated must be crossed with lines that are GMS/GMS or GMS/gms, giving rise to mixed populations of sterile and fertile plants.

The trait cytoplasmic male sterility (CMS) differs from GMS in that the failure to produce pollen is mediated by genes that do not segregate in a Mendelian fashion; instead, they display a pattern of strict maternal inheritance. The difference in inheritance patterns between GMS and CMS systems can be attributed to the later being caused by aberrant genes found in the genome of the mitochondria, an organelle passed to progeny strictly from the maternal parent in the majority of plant species. It is this maternal inheritance of the CMS trait, which when coupled to an appropriate nuclear-encoded restorer-of-fertility (Rf) gene at the time of hybrid seed production, that has made CMS systems of great value to the hybrid seed industry. CMS-based systems of hybrid seed production require three lines: (1) the CMS line possessing the mitochondrial gene mutation; (2) a maintainer line in the same inbred background as the CMS line, but in a normal, non-mutant cytoplasm to serve as the pollen parent to enable propagation of the CMS line; and (3) a restorer line in a different inbred background that possesses dominant Rf genes capable of overcoming the mitochondrial gene defect and thus restore the ability of a plant containing the CMS mutation to produce pollen.

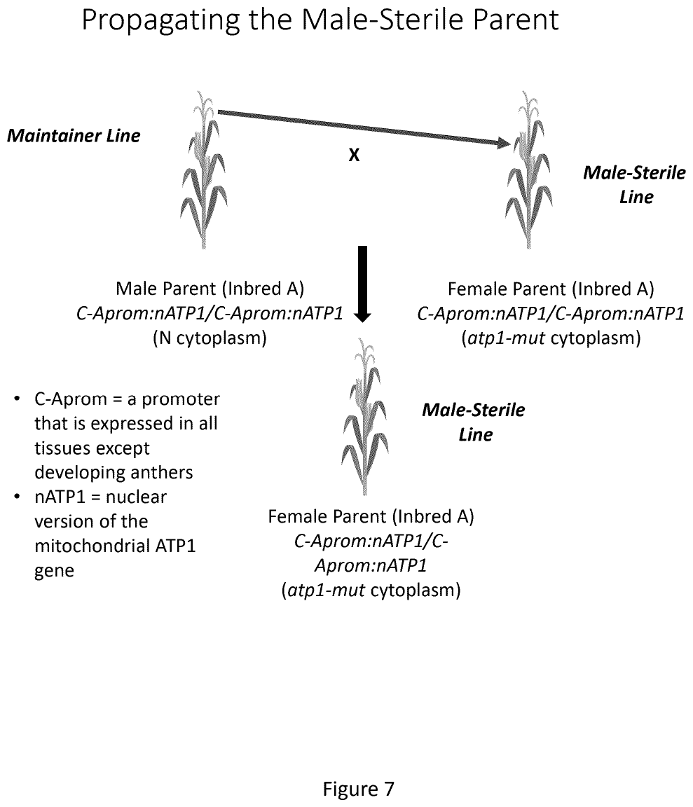

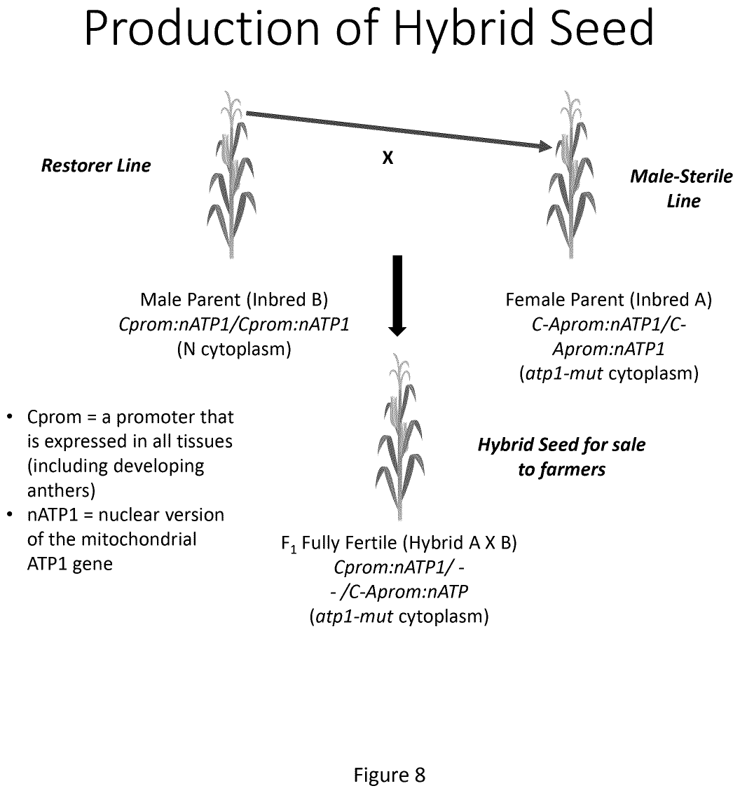

By transferring the function of an essential mitochondrial gene to the nucleus, it could be possible to produce plants that would be viable even if after the endogenous mitochondrial gene had been eliminated. Further, depending on the specific promoters chosen to drive the expression of the nuclear version of the essential mitochondrial gene, a novel CMS-based hybrid seed production system could be developed. Briefly, a maintainer line could be developed by placing the nuclear version of the mitochondrial gene under the control of a promoter that is active in all plant tissues expect developing anthers, and a restorer line could be generated by placing the same gene under the transcriptional control of a promoter that was active in all plant tissues and cells. The rationale of this strategy, using the essential mitochondrial gene mtATP1 as the gene target and maize as the recipient background, is depicted in FIGS. 7 and 8.

As shown in FIG. 7, the maintainer line possesses a version of mtATP1 that has been redesigned for expression in the nucleus (designated nATP1) and placed under the control of a promoter that is constitutively expressed in all cell types and development stages of the plant except those involve in anthesis (designated C-Aprom, for “constitutive” (C) minus “anthesis” (A)). The male-sterile line is in the identical inbred background as the maintainer (Inbred A), but lacks the endogenous mtATP1 gene. Inbred A with the atp1-mut cytoplasm can thus be propagated indefinitely via pollination by the maintainer line containing C-Aprom:nATP1. As depicted in FIG. 8, the restorer line possesses an nATP1 construct under the transcriptional control of a promoter that is active in all cell and tissue types of the plant βprom:nATP1), but in a different inbred background (Inbred B) that demonstrates a high level of heterosis when combined with Inbred A. When the restorer line B is crossed with the male-sterile line A, F₁ seed is produced that should be fertile and can be grown commercially, as the dominant Cprom:nATP1 construct will restore fertility to the A×B progeny.

B. Results (Methods and Materials Included Simultaneously)

For proof-of-concept, tobacco was chosen as the plant species of choice due to its amenability to genetic manipulation, and the aforementioned mtATP1 gene was selected as the essential mitochondrial gene. As a candidate promoter for C-Aprom, an enhanced CaMV 35S (e35S) promoter was selected. The CaMV 35S promoter is the best characterized and most widely used plant promoter, both in basic transgenic plant studies as well as commercial deployment. Despite the fact that the CaMV 35S promoter is commonly described in the literature as being a “strong constitutive promoter”, in studies using the CaMV 35S promoter in conjunction with a reporter gene in transgenic tobacco, tomato and potato, expression was not detectable in the tapetal layer of developing anthers or the sporogenic cells surrounded by the tapetum (Plegt and Bino, 1989 Mol. Gen. Genet. 216: 321-327). The lack of e35S CaMV activity in developing anthers was also documented in maize, a phenomenon that was exploited by Monsanto in the development of a seed production system referred to as the Roundup Hybridization System. Thus, an e35S CaMV promoter was selected as the candidate C-Aprom promoter for the proof-of-concept experiments.

As a candidate for a Cprom promoter that would be constitutively active throughout the entire plant, the promoter of the gene that drives the expression of the β-subunit of the mitochondrial ATPase complex (encoded by the nuclear gene ATP2) was selected. This choice was based on the rationale that the α- and β-subunits of the ATPase complex are similar in size, share primary amino acid sequence homology, and are found in equal stoichiometries within the complex (three α- and three β-subunits per enzyme). Therefore, it stood to reason that if the α-subunit were under the same regulatory control as its partner the β-subunit, it would be sufficient to meet the ATPase demands of the cell.

B1. Generation of the nATP1 Construct

When converting a plant mitochondrial gene to a nuclear gene it is important to accommodate for the following two phenomena: (1) translation of mitochondrial transcripts is prokaryotic-like in nature, so it is advisable to have the gene resynthesized using codons optimal for expression in the nucleus; and (2) the process known as RNA editing is prevalent in plant mitochondria, meaning that the functional transcript does not always match the DNA sequence from which the transcript was derived (reviewed in Small et al. 2020). To assure proper function as a nuclear gene, it is therefore important to ensure that the codons of the repurposed mitochondrial gene match that of the mature transcripts of the gene, not the genomic version of the gene. Organellar RNA editing primarily consists of converting select “C” residues to “U” residues. GenBank accession number BA000042 contains the complete DNA sequence of the tobacco mitochondrial genome. Importantly, based on an analysis of the corresponding transcriptome, it also includes all the positions where Cs are converted to Us in the mature transcripts. In the mtATP1 gene there are a total of six locations in the transcript where RNA editing causes the mature transcript to differ from that found in the mtATP1 gene per se. The sequence of mature tobacco mtATP1 cDNA sequence (using T nucleotides as opposed to the U nucleotides of the RNA transcript) is described in the description of SEQ ID NO: 19.

As an initial step in converting mtATP1 from a mitochondrial gene to a nuclear gene, the sequence shown in SEQ ID NO: 19 was synthesized using codon optimization according to tobacco nuclear genes. In addition to the mtATP1 sequence, the custom-synthesized sequence was also designed such that the ATP1 protein would be fused at the N-terminus to the mitochondrial transit peptide of the tobacco I3-subunit of the ATPase in addition to the first 12 amino acids of the mature I3-subunit protein. These sequences were selected based on previous research that demonstrated in Nicotiana plumbaginifolia that the I3-subunit ATPase transit peptide when coupled with the first 12 amino acids of the mature product could efficiently transport foreign proteins into the mitochondria. Finally, the synthesized sequence also included 889 bp of sequence 3′ of the tobacco ATP2 gene (GenBank accession NCAA01008649) in order to capture 3′-UTR, polyadenylation, and/or other regulatory sequences that may be found in that region of the gene. The complete DNA sequence of the nATP1 construct is shown in SEQ ID NO: 20.

B2. Generation of 35S:nATP1 and β-Prom:nATP1 Constructs

An enhanced CaMV 35S promoter was amplified from the CAMBIA vector pCAMBIA2300 using primers that placed a SalI restriction site at the 5′ end of the sequence and a BamHI restriction site at the 3′ end. This fragment was joined to the nATP construct at the common BamHI restriction site to create the construct designated 35S:nATP1 as shown in SEQ ID NO: 21.

Because the tobacco ATP2 promoter has not been characterized, a fragment was isolated that was predicted would be likely to contain the promoter based on previous observations that most of the elements comprising plant promoters are found with 2 kb of the initiation of transcription site. A DNA fragment containing the predicted ATP2 promoter (including 101 bp of 5-UTR based on corresponding EST sequences) was isolated via PCR using total genomic tobacco DNA and primers directed against the 2.2 kb region upstream of the ATG start codon (as found in GenBank accession AWOJ01393114). The PCR primers were designed to create a SalI restriction site at the 5′ and a BamHI site at the 3′ end. The resulting fragment was joined to the nATP1 construct at the common BamHI restriction site to create the construct designated βprom:nATP1 as shown in SEQ ID NO: 22.

B3. Development of 35S:nATP1 and β-prom:nATP1 tobacco lines

The unique SalI and KpnI restriction sites that flank constructs 35S:nATP1 and βprom:nATP1 facilitated the cloning of the two constructs into the same sites in the plant expression vector pCAMBIA1300. The pCAMBIA1300/35S:nATP1 and pCAMBIA1300/βprom:nATP1 vectors were transformed into Agrobacterium tumefaciens strain LBA4404 and introduced into tobacco leaf discs using standard transformation protocols, with hygromycin as the selection agent. To facilitate the generation of transgenic tobacco lines that would be fixed for the transgene in a single generation, leaf tissue of haploid plants of tobacco cultivar K326 was used as the explant. Eighteen independent transformed haploid events were recovered for the βprom:nATP1 construct, and 19 events were recovered that contained 35S:nATP1.

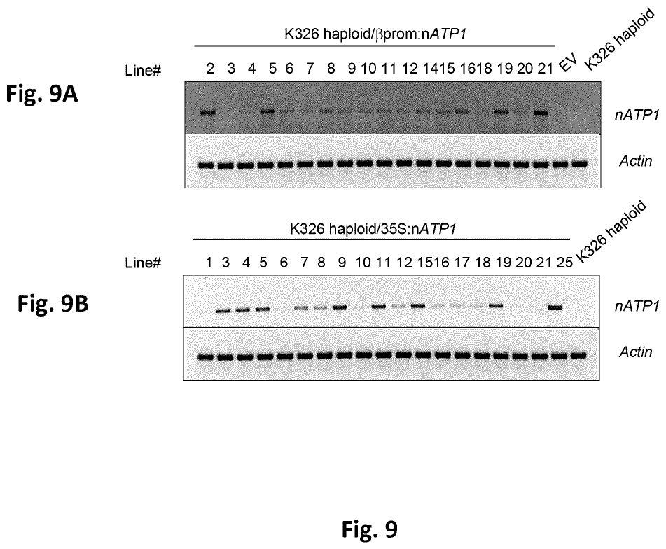

To determine which individual haploid individuals displayed the highest level of transgene expression, RNA was isolated from leaf tissue of young transformed plants according to standard protocols. First-strand cDNA was produced from the total RNA preparations using reverse transcriptase, then assayed using semi-quantitative PCR by conducting a non-saturating number of PCR cycles (20) on each sample and running the products on agarose gels. The nATP1-specific primers used for these assays were: 5′-GGATCCACTATGGCTTCTCGGAG-3′ (SEQ ID NO: 23) (forward); and 5′-CTATAGATCTTCAGATGAATGCG-3′ (SEQ ID NO: 24). As a control, first-strand cDNA was also amplified in a similar manner using primers specific to a tobacco actin gene (forward primer=5′-AGCACCCTGTTCTTCTCAC-3′ (SEQ ID NO: 25); reverse primer=5′-GTCAAGCTCCTGCTCGTAG-3′(SEQ ID NO: 26)). The semi-quantitative PCR results are shown in FIG. 9. Based on these results, K326 βprom:nATP1 haploid plants #2, #16 and #21, and K326 35S:nATP1 haploid plants #9, #11 and #25 were chosen to advance for chromosome doubling. Fully fertile, doubled haploids homozygous for the transgene insertions were produced from the haploid plants using the midvein culturing technique.

B4. Elimination of the mtATP1 Gene in K326/35S:nATP1 and K326/βProm:nATP1 Lines

Doubled haploid K326 lines 35S:nATP1 #25 and βprom:nATP1 #21 were transformed with plant transformation binary vectors containing the engineered ATP 5-6 and ATP 7-8 MTEM constructs. For the sake of simplicity, the binary vector possessing only the ATP 5-6 construct shown in FIG. 6B will be referred to as SP2289, the binary vector contain only ATP 7-8 (FIG. 6C) will be called SP4693, and the binary vector possessing both ATP 5-6 and ATP 7-8 MTEM constructs (FIG. 6A) will be referred to as SP3379. All three of the binary vectors were introduced by Agrobacterium-mediated transformation into K326 line βprom:nATP1 #21 using kanamycin selection; only SP3379 was transformed into the K326 35S:nATP1 #25 background. The number of kanamycin-resistant transformed plants recovered in each experiment was as follows: 35S:nATP1/SP3379=15; βprom:nATP1/SP3379=9; βprom:nATP1/SP2289=6; and βprom:nATP1/SP4693=7. As a control, a K326 empty vector (EV) doubled haploid individual (transformed with pCAMBIA1300 lacking an nATP1 transgene) was also transformed with SP3379, and 9 plants were recovered.

In order to determine whether the mtATP1 gene had been altered in any of the individuals transformed with MTEM constructs designed to target the gene, PCR amplifications were conducted using primer pairs that flanked both the ATP 5-6 and ATP 7-8 recognition sites. Specifically, the forward primer 5′-CAAGTGGATGAGATCGGTCG-3′ (SEQ ID NO: 27) was paired with the reverse primer 5′-ACTGACAGATAAGCCGACGT-3′ (SEQ ID NO: 28), producing a 1050 bp PCR product when the wild type mtATP1 gene is amplified. As a control, primers specific for the mitochondrial gene mtATP6 (forward primer 5′-ATCCGGGCCTTAATCCTTGC-3′ (SEQ ID NO: 29) and reverse primer 5′-TGCGAGGGGAAAACTTTTGT-3′ (SEQ ID NO: 30)) were also included in each PCR reaction. The mtATP6-specific primers were expected yield a 511 bp amplification product.

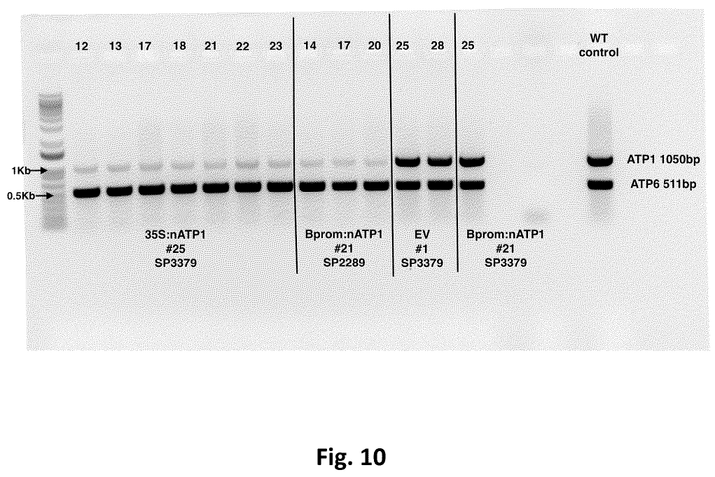

Total DNA was isolated from young leaf tissue for each of the T₀ plants generated in these experiments and evaluated by PCR using the mtATP1- and mtATP6-specific primers. When total DNA from WT tobacco was amplified using these primers, bands consistent with the predicted sizes of 1050 bp (mtATP1) and 511 bp (mtATP6) were observed in similar stoichiometries. Interestingly, an exceptionally high percentage of 35S:nATP1 and βprom:nATP1 plants transformed with the MTEM constructs displayed a band of the expected size for mtATP1 that was of a greatly reduced in stoichiometry in comparison to mtATP6. Typical examples are shown in FIG. 10. Several alternative mtATP1-specific primer pairs were also tested, and in each case, a fainter sub-stoichiometric band corresponding in size to that expected for mtATP1 was observed. There are two plausible explanations for these observations: (1) the cleavage of mtATP1 by the MTEM enzymes yielded heterogenous populations of mitochondrial genomes, the majority of which lacked mtATP1 while a minority retained the gene; or (2) the MTEM enzymes mediated the complete elimination of mtATP1 from all mitochondrial genomes, and the faint amplification products are false positives corresponding to nuclear-encoded mitochondrial DNA sequences (NUMTs). Over the course of evolution, large portions of the mitochondrial genome have become incorporated into the nuclear genome. These presumably nonfunctional sequences are referred to as NUMTs, and polymorphisms that have accumulated over time can differentiate a true mitochondrial gene from an NUMT. When BLASTN searches were conducted on draft genome sequences of Nicotiana tabacum using mtATP1 as the query sequence, over 30 scaffolds or contigs were found that shared greater than 93% sequence identity to the genuine mtATP1 sequence. The possibility that amplification of NUMTs provides the explanation for the results shown in FIG. 10 is given further credence by the study of Arimura et al. (2020 Plant J. 104: 1459-1471) who attempted to eliminate one of two redundant mtATP6 genes that are found in the mitochondrial genome of Arabidopsis. In that study, the presence of NUMTs with high sequence homology to mtATP6 genes prevented them from establishing whether the loss of one isoform (designated atp6-1 in that paper) was driven to homoplasmy based solely on PCR analysis.

In order to test whether the sub-stoichiometric bands that appear in the PCR analyses of 35S:nATP1 and βprom:nATP1 plants that have been transformed with MTEM constructs are legitimate mtATP1 sequences versus ATP1-like NUMTs, the low level PCR products from two independent 35S:nATP1/SP3379 individuals (35S:nATP1/SP3379 #8 and 35S:nATP1/SP3379 #16) were cloned into vector pCR-Blunt (Invitrogen). As a control, the PCR amplification products from a WT tobacco plant using the same mtATP1-specific primers were also cloned into pCR-Blunt. A total of 23 independently cloned PCR products were sequenced from each of the three backgrounds tested. None of the amplification products from plants 35S:nATP1/SP3379 #8 or 35S:nATP1/SP3379 #16 were 100% identical to the WT mtATP1 sequence. Instead each contained polymorphisms that could also be found in one or more tobacco NUMT. In contrast, 20 out of 23 cloned PCR products amplified from the WT tobacco plant were 100% identical to the legitimate mtATP1 gene; the other three displaying polymorphisms found in NUMTs. The results of these experiments strongly suggest that no intact copies of the endogenous tobacco mtATP1 gene remain in 35S:nATP1 and βprom:nATP1 plants that have been transformed with the MTEM constructs directed against mtATP1 and display only faint sub-stoichiometric PCR products when amplified with primers designed against mtATP1.

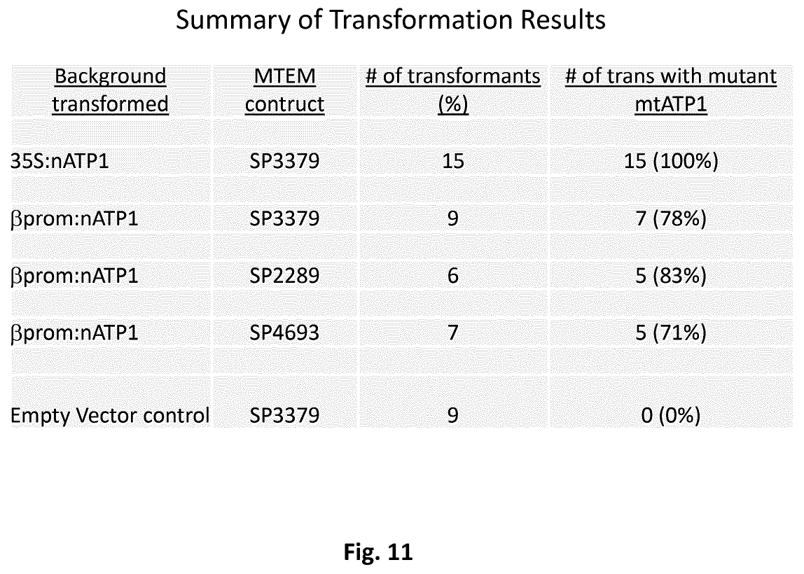

A summary of the transformation results is shown in FIG. 11. In the 35S:nATP1 background transformed with SP3379, 100% of the transformed events (15/15) appeared to lack mtATP1. In the βprom:nATP1 background as well, the majority of the transformants recovered were missing mtATP1, regardless of which of the 3 MTEM constructs were introduced. In contrast, all of the EV control plants transformed with SP3379 showed mtATP1 amplification products in equal stoichiometry with mtATP6, similar to the WT control (see FIG. 10 for two representative examples). Given that the elimination of mtATP1 gene function in a tobacco plant that does not have a compensating nATP1 gene introduced into the nucleus would be predicted to be lethal, it is expected that the only EV/SP3379 plants that could be recovered would be those in which the MTEM constructs are not effectively expressed. Finally, although combining the ATP 5/6 with ATP7/8 constructs within a single vector gave rise to the possibility of the compatible sticky ends relegating to form a deletion mutation with 674 bp perfectly excised (as shown at a very low level in the protoplast experiments; FIG. 4), no such product was observed in any of the 35S:nATP1/SP3379 or βprom:nATP1/SP3379 transformants recovered.

B5. Phenotypic Analysis of ΔmtATP1 Plants

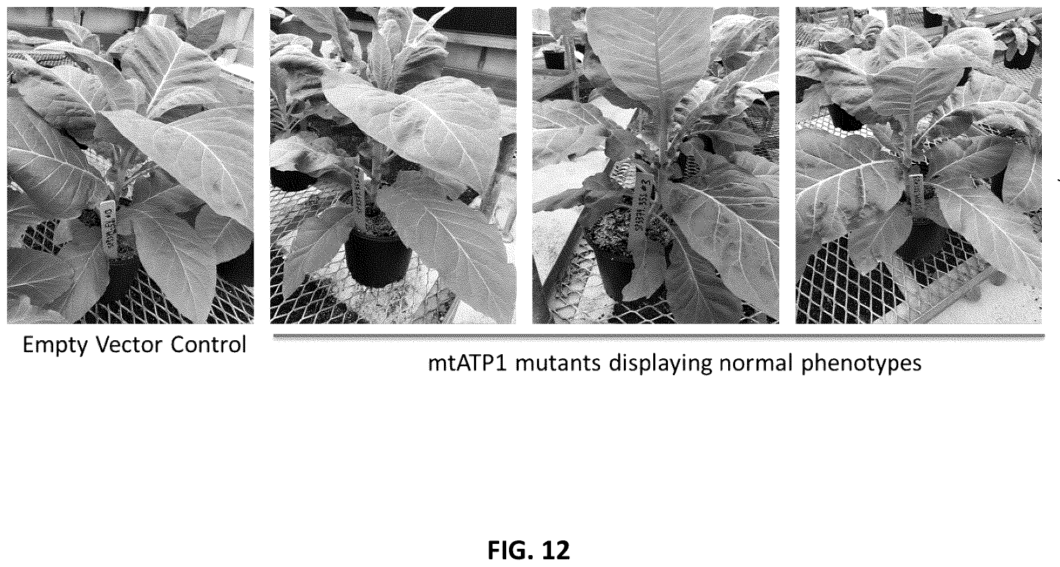

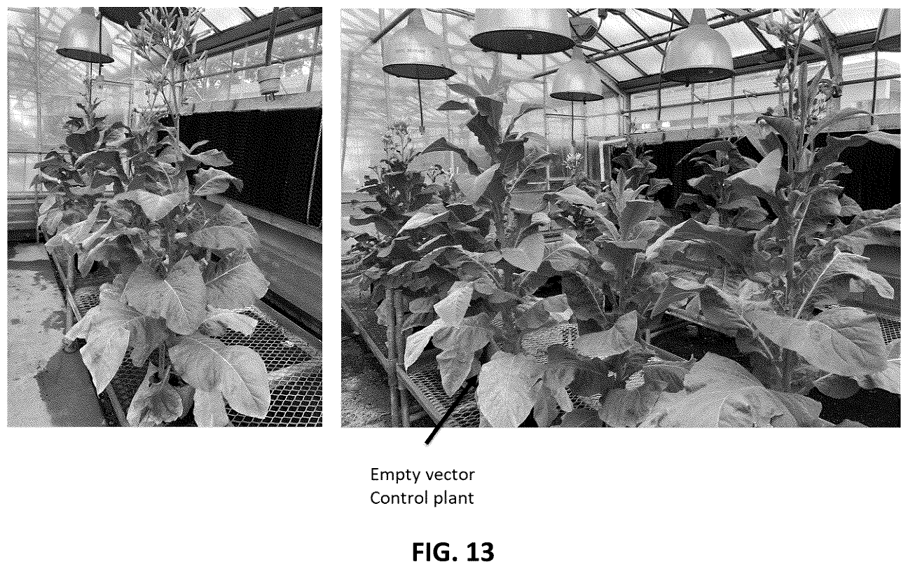

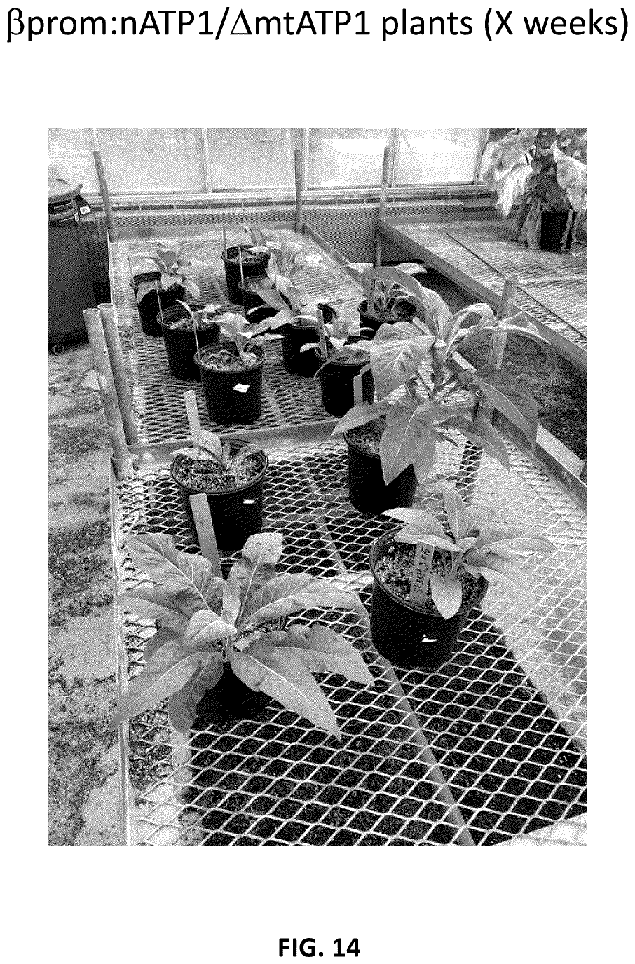

Once T₀ plants had successfully rooted in culture media, they were transferred to soil and placed on growth racks with supplemental lighting. Once the plants were approximately 10-15 cm in height, they were transferred to large pots and grown to maturity in a greenhouse. The majority of the plants that appeared to lack mtATP1 (termed ΔmtATP1) in the 35S:nATP1 background grew and developed in a manner similar to empty vector control plants. Pictures of representative plants approximately six weeks and ten weeks after transfer to soil are shown in FIGS. 12 and 13, respectively. In contrast to the ΔmtATP1 mutants in 35S:nATP1, the ΔmtATP1 individuals in the βprom:ATP1 background grew much more slowly than normal tobaccos. This is exemplified in FIG. 14 which shows βprom:ATP1/ΔmtATP1 individuals approximately 12 weeks after having been transplanted to soil. These results suggest that during the vegetative growth stage, the enhanced CaMV 35S promoter is able to drive the expression of nATP1 in a manner that fully compensates for the absence of mtATP1. Although the βprom utilized in this study (derived from the tobacco ATP2 gene) appeared to express nATP1 at levels sufficient for survival, it did not fully compensate for the ΔmtATP1 mutation as evidenced by the impaired growth phenotype. Therefore, it is likely that a stronger promoter than βprom will be required to serve as the hypothetical Cprom promoter depicted in FIG. 8 for the development of restorer lines in the novel hybrid seed production system.

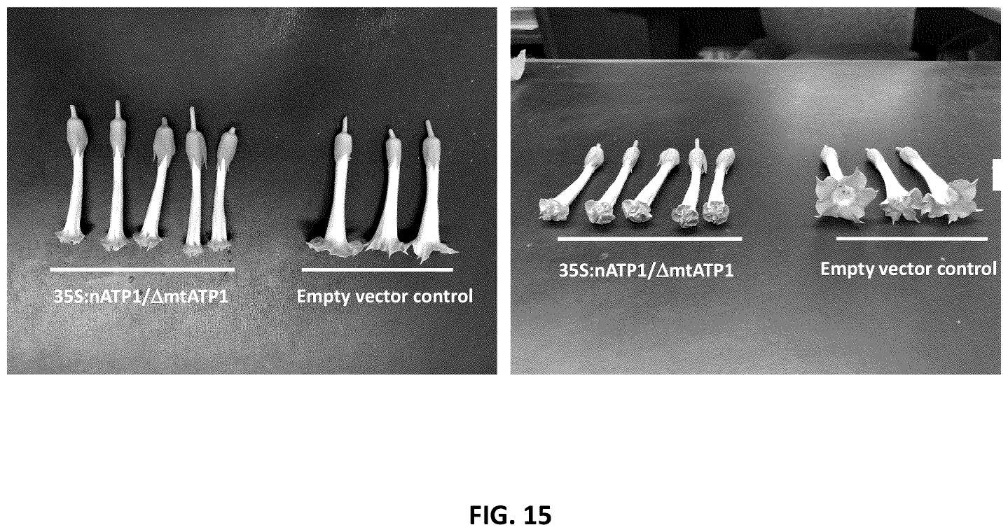

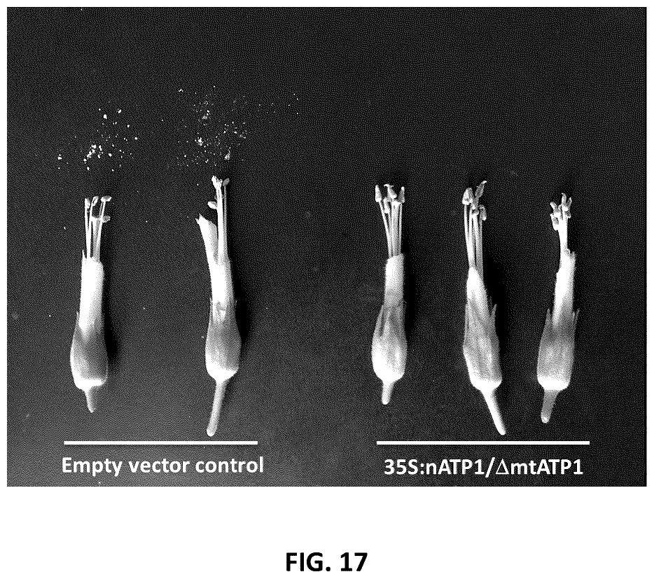

Given that 35S:nATP1/ΔmtATP1 plants looked normal during vegetative growth, the enhanced CaMV 35S promoter appeared to be a viable C-Aprom candidate for developing the male-sterile and maintainer lines shown in FIGS. 7 and 8. As flowers began to form on these plants, they were somewhat different in appearance than normal tobacco flowers. As shown in FIG. 15, the petals of 35S:nATP1/ΔmtATP1 plants appeared more narrow throughout and less expanded at the opening. In keeping with our prediction that the CaMV 35S promoter is not active during certain stages of anther development, no evidence was observed of pollen being formed on any of the 35S:nATP1/ΔmtATP1 plants that were generated in this study. As shown in the representative picture in FIG. 17, when one taps a normal tobacco anther at maturity on a black background, an abundance of pollen is observed. No such evidence was seen from 35S:nATP1/ΔmtATP1 anthers. Likewise, pollen staining protocols also failed to detect the presence of viable pollen in 35S:nATP1/ΔmtATP1 plants. The failure to produce pollen supports the conclusion that 35S:nATP1/ΔmtATP1 plants display a CMS phenotype.

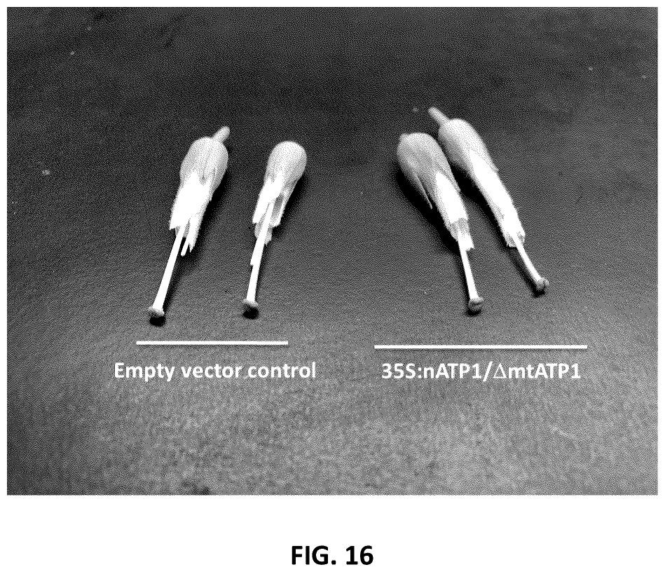

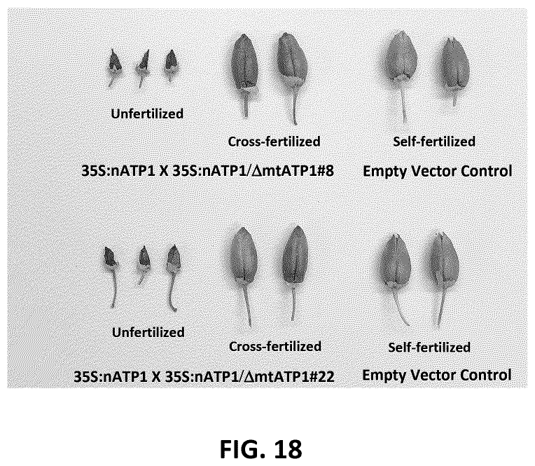

Also in keeping with the hypothesis that the CaMV 35S promoter is deficient in cell types specific to male, but not female reproductive development, the pistils and stigmas found in the flowers of 35S:nATP1/ΔmtATP1 individuals appeared to be normal (FIG. 16). To further assess the viability of female fertility, pollen from wild type and 35S:nATP1 plants (in a normal cytoplasm) was applied to the stigmas of 35S:nATP1/ΔmtATP1 individuals. Pod development appeared to be normal in flowers where WT X 35S:nATP1/ΔmtATP1 and 35S:nATP1 X 35S:nATP1/ΔmtATP1 crosses were made. In contrast, the pods remained small and undeveloped in unfertilized flowers on these same plants. FIG. 18 shows representative mature seed pods from two independent 35S:nATP1/ΔmtATP1 plants. The pods from unfertilized flowers from plants 35S:nATP1/ΔmtATP1 #8 and 35S:nATP1/ΔmtATP1 #22 are small, showing minimal ovary development. The fertilized flowers on these same plants, however, displayed normal ovary development, giving rise to normal looking tobacco pods as seen in comparison to the self-pollinated pods from an empty vector control plant.

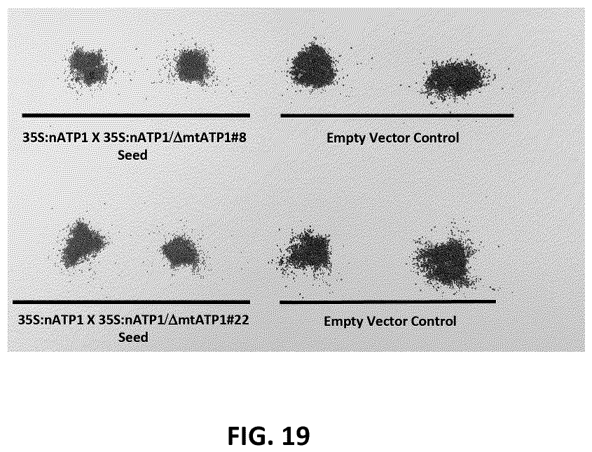

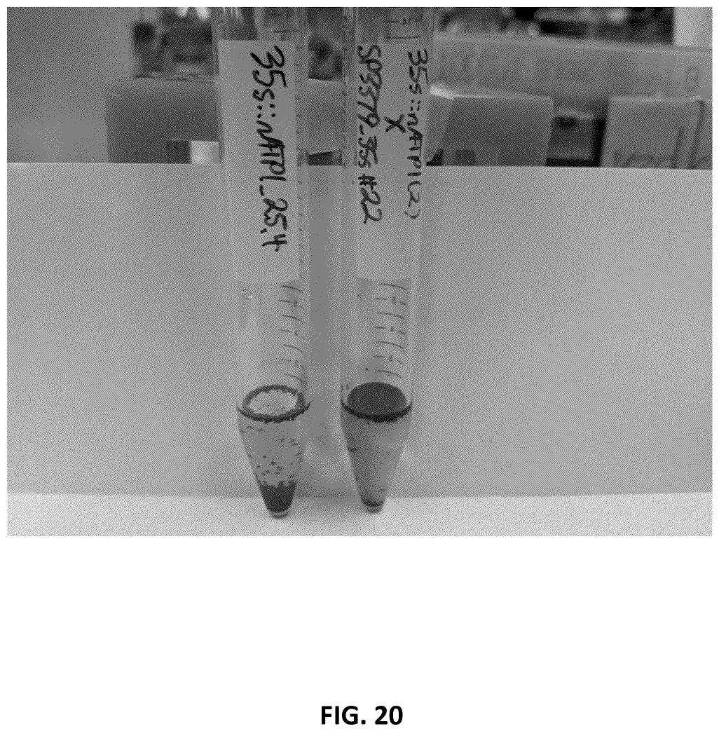

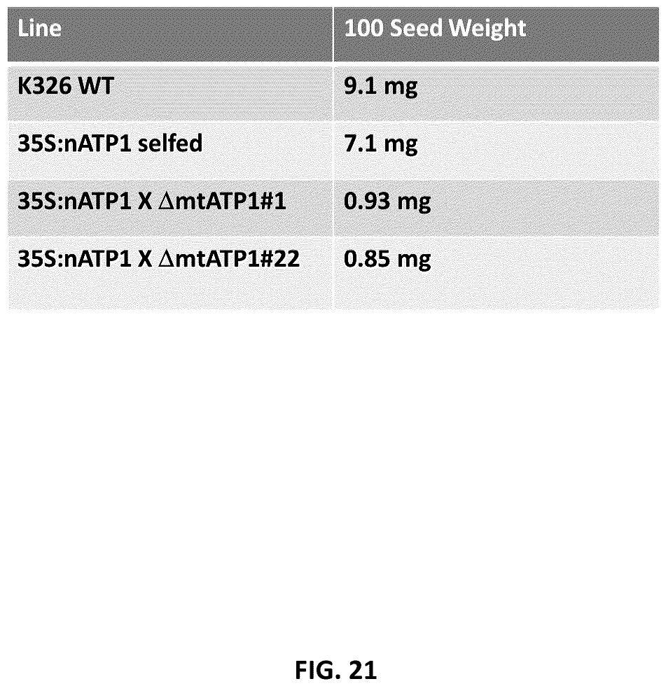

Although the outward appearance of 35S:nATP1 X 35S:nATP1/ΔmtATP1 pods looked the same as EV control pods, when they were opened and the seeds allowed to disperse, there were noticeable differences. As shown in FIG. 19, the seeds from 35S:nATP1 X 35S:nATP1/ΔmtATP1 pods were smaller and a lighter shade of brown than the normal tobacco seeds observed from the EV control pods. Furthermore, tobacco seeds from plants that had normal cytoplasms mostly sank when placed in water, in contrast to the seed-like structures produced in 35S:nATP1 X 35S:nATP1/ΔmtATP1 pods that mostly floated to the top (FIG. 20). These results suggest that the seed-like particles produced in these crosses were less dense than normal tobacco seeds. To explore this further, the average weight of 100 seeds from WT K326, a 35S:nATP1 fertile plant, and two 35S:ATP1 X 35S:nATP1/ΔmtATP1 crosses was calculated. As shown in FIG. 21, the average 100 seed weight observed from plants with normal cytoplasms was 8-11 times heavier than that observed from with crosses onto plants with mutant (ΔmtATP1) cytoplasms. Finally, thousands of seeds produced from dozens of crosses from male-fertile tobacco plants onto 35S:nATP1/ΔmtATP1 plants were planted onto soil and/or solid growth media and failed to observe even one seed that germinated. These results suggest that in addition to anther development, the enhanced CaMV 35S promoter also lacks sufficient activity during some stage of seed development. Although a comprehensive analysis of CaMV 35S promoter activity in every cell and tissue type of tobacco has not been reported, in cotton such an analysis revealed that this promoter showed little to no activity during the early stages of embryo and endosperm development (Sunilkumar et al., 2002). The failure of 35S:nATP1/ΔmtATP1 plants to produce viable seed when fertilized using pollen from 35S:nATP1 plants is consistent with the interpretation that the CaMV 35S promoter is minimally expressed during tobacco seed development as well, leading to seed abortion due to an insufficient supply of ATP1 to the mitochondria.

C. Conclusions

The experiments described herein demonstrate that an essential plant mitochondrial gene can be redesigned as a nuclear gene to compensate for the complete loss of function of that gene from the mitochondrial genome. This observation alone has not been previously reported. The feasibility of this concept was demonstrated by repurposing the essential gene mitochondrial gene mtATP1 in a manner that enabled it to function properly as a nuclear gene, followed by knocking out the endogeneous mtATP1 gene using custom-designed MTEM constructs. These experiments were originally conducted under the hypothesis that genetic manipulations of this nature could be exploited to develop new CMS-based hybrid seed production systems as detailed in FIGS. 7 and 8. The results reported here demonstrated the overall technical feasibility of this approach. The observation that the nATP1 construct described here could fully compensate for the absence of mtATP1 in the mitochondria during vegetative growth and female reproduction, and yield a CMS phenotype, serves as convincing proof-of-concept for the principles outlined in FIGS. 7 and 8.

Reworking the envisioned hybrid seed production system to the extent where it could be commercially viable should simply be a matter of adjusting the promoters utilized to drive the expression of the nATP1 construct. For example, a construct consisting of nATP1 driven by an early embryogenesis-specific promoter, when place on the same vector as the 35S:nATP construct, would likely meet the requirement of the C-Aprom of FIGS. 7 and 8. Furthermore, replacing βprom with a stronger constitutive and/or ubiquitous promoter such the soybean ubiquitin promoter when applied to dicots, or the rice Act1 promoter when implemented in monocots should provide sufficient efficacy to serve in the creation of the restorer line depicted in FIG. 8.

The observation that fertilization of 35S:nATP1/ΔmtATP1 plants with pollen from WT or 35S:nATP plants resulted in normal ovary development with aborted seed production suggests an alternative application of mitochondrial genome editing technologies. Stenospermocarpy is the biological phenomenon in plants where fertilization stimulates normal ovary/fruit development in the presence of incomplete seed development. Stenospermocarpy has been successfully exploited for the commercial production of “seedless” fruits, with seedless watermelons and seedless grapes being two of the most popular applications. Fertilized 35S:nATP1/ΔmtATP1 plants showed all of the hallmarks of stenospermocarpy in that the ovary (pod) became fully developed, but produced only light weight, less dense seed remnants. When extrapolated to fruiting crop species such as tomatoes and blackberries, or a vegetable species like cucumbers, deployment of the 35S:nATP1/ΔmtATP1 technology would be expected to have novel application in the production of seedless fruits and vegetables.

In addition to using mitochondrial genome editing and transfer of gene function to the nucleus for the aforementioned CMS-based hybrid seed system and the production of seedless fruits and vegetables via stenospermocarpy, our results also demonstrate that the 35S:nATP1/ΔmtATP1 technology could also be applied as a very effective means of transgene containment for horticultural or crop species that are routinely propagated via micropropagation or cuttings. 35S:nATP1/ΔmtATP1 tobacco plants cannot transmit any of their genetic information through either the pollen or the seed, making it an ideal system for preventing the uncontrolled dissemination of transgenes into the environment. There are numerous horticultural species in particular that are propagated by the industry using stem cuttings. Should a novel flower color or desirable disease resistance trait, for example, be introduced using a transgene gene, regulatory and public perception concerns may arise concerning the spread of the transgene into the wild through either the pollen or seed. By introducing the 35S:nATP1/ΔmtATP1 technology together with the transgene of interest, one could prevent the spread of the transgene due to the plant's inability to produce pollen or seeds.

1. An engineered meganuclease that binds and cleaves a recognition sequence comprising SEQ ID NO: 1 in a plant mitochondrial ATP synthase 1 (mtATP1) gene, wherein said engineered meganuclease comprises a first subunit and a second subunit, wherein said first subunit binds to a first recognition half-site of said recognition sequence and comprises a first hypervariable (HVR1) region, wherein said second subunit binds to a second recognition half-site of said recognition sequence and comprises a second hypervariable (HVR2) region, wherein said HVR1 region comprises an amino acid sequence having at least 80% sequence identity to an amino acid sequence corresponding to residues 24-79 of SEQ ID NO: 3, and wherein said HVR2 region comprises an amino acid sequence having at least 80% sequence identity to an amino acid sequence corresponding to residues 215-270 of SEQ ID NO: 3.

2. The engineered meganuclease of claim 1, wherein said engineered meganuclease is a mitochondria-targeted engineered meganuclease (MTEM) that comprises said engineered meganuclease attached to a mitochondrial transit peptide (MTP).

3. The MTEM of claim 2, wherein said HVR1 region comprises one or more residues corresponding to residues 24, 26, 28, 30, 32, 33, 38, 40, 42, 44, 46, 68, 70, 75, and 77 of SEQ ID NO: 3.

4. The MTEM of claim 2 or claim 3, wherein said HVR1 region comprises residues 24-79 of SEQ ID NO: 3.

5. The MTEM of any one of claims 2-4, wherein said first subunit comprises an amino acid sequence having at least 80% sequence identity to residues 7-153 of SEQ ID NO: 3.

6. The MTEM of any one of claims 2-5, wherein said first subunit comprises a residue corresponding to residue 80 of SEQ ID NO: 3.

7. The MTEM of any one of claims 2-6, wherein said first subunit comprises residues 7-153 of SEQ ID NO: 3.

8. The MTEM of any one of claims 2-7, wherein said HVR2 region comprises one or more residues corresponding to residues 215, 217, 219, 221, 223, 224, 229, 231, 233, 235, 237, 259, 261, 266, and 268 of SEQ ID NO: 3.

9. The MTEM of any one of claims 2-8, wherein said HVR2 region comprises residues 215-270 of SEQ ID NO: 3.

10. The MTEM of any one of claims 2-9, wherein said second subunit comprises an amino acid sequence having at least 80% sequence identity to residues 198-344 of SEQ ID NO: 3.

11. The MTEM of any one of claims 2-10, wherein said second subunit comprises a residue corresponding to residue 210 of SEQ ID NO: 3.

12. The MTEM of any one of claims 12-11, wherein said second subunit comprises a residue corresponding to residue 271 of SEQ ID NO: 3.

13. The MTEM of any one of claims 2-12, wherein said second subunit comprises residues 198-344 of SEQ ID NO: 3.

14. The MTEM of any one of claims 2-13, wherein said engineered meganuclease is a single-chain meganuclease comprising a linker, wherein said linker covalently joins said first subunit and said second subunit.

15. The MTEM of any one of claims 2-14, wherein said engineered meganuclease comprises an amino acid sequence having at least 80% sequence identity to SEQ ID NO: 3.

16. The MTEM of any one of claims 2-15, wherein said engineered meganuclease comprises an amino acid sequence of SEQ ID NO: 3.

17. The MTEM of any one of claims 2-16, wherein said engineered meganuclease is encoded by a nucleic sequence having at least 80% sequence identity to a nucleic acid sequence of SEQ ID NO: 4.

18. The MTEM of any one of claims 2-17, wherein said engineered meganuclease is encoded by a nucleic acid sequence of SEQ ID NO: 4.

19. An engineered meganuclease that binds and cleaves a recognition sequence comprising SEQ ID NO: 2 in a plant mtATP1 gene, wherein said engineered meganuclease comprises a first subunit and a second subunit, wherein said first subunit binds to a first recognition half-site of said recognition sequence and comprises a first hypervariable (HVR1) region, wherein said second subunit binds to a second recognition half-site of said recognition sequence and comprises a second hypervariable (HVR2) region, wherein said HVR1 region comprises an amino acid sequence having at least 80% sequence identity to an amino acid sequence corresponding to residues 24-79 of SEQ ID NO: 5, and wherein said HVR2 region comprises an amino acid sequence having at least 80% sequence identity to an amino acid sequence corresponding to residues 215-270 of SEQ ID NO: 5.

20. The engineered meganuclease of claim 19, wherein said engineered meganuclease is an MTEM that comprises said engineered meganuclease attached to an MTP.

21. The MTEM of claim 20, wherein said HVR1 region comprises an amino acid sequence having at least 80% sequence identity to residues 24-79 of SEQ ID NO: 5.

22. The MTEM of claim 20 or claim 21, wherein said HVR1 region comprises one or more residues corresponding to residues 24, 26, 28, 30, 32, 33, 38, 40, 42, 44, 46, 68, 70, 75, and 77 of SEQ ID NO: 5.

23. The MTEM of any one of claims 20-22, wherein said HVR1 region comprises residues corresponding to residues 24, 26, 28, 30, 32, 33, 38, 40, 42, 44, 46, 68, 70, 75, and 77 of SEQ ID NO: 5.

24. The MTEM of any one of claims 20-23, wherein said HVR1 region comprises residues 24-79 of SEQ ID NO: 5.

25. The MTEM of any one of claims 20-24, wherein said first subunit comprises an amino acid sequence having at least 80% sequence identity to residues 7-153 of SEQ ID NO: 5.

26. The MTEM of any one of claims 20-25, wherein said first subunit comprises a residue corresponding to residue 80 of SEQ ID NO: 5.

27. The MTEM of any one of claims 20-26, wherein said first subunit comprises residues 7-153 of SEQ ID NO: 5.

28. The MTEM of any one of claims 20-27, wherein said HVR2 region comprises an amino acid sequence having at least 80% sequence identity to residues 215-270 of SEQ ID NO: 5.

29. The MTEM of any one of claims 20-28, wherein said HVR2 region comprises one or more residues corresponding to residues 215, 217, 219, 221, 223, 224, 229, 231, 233, 235, 237, 259, 261, 266, and 268 of SEQ ID NO: 5.

30. The MTEM of any one of claims 20-29, wherein said HVR2 region comprises residues corresponding to residues 215, 217, 219, 221, 223, 224, 229, 231, 233, 235, 237, 259, 261, 266, and 268 of SEQ ID NO: 5.

31. The MTEM of any one of claims 20-30, wherein said HVR2 region comprises residues 215-270 of SEQ ID NO: 5.

32. The MTEM of any one of claims 20-31, wherein said second subunit comprises an amino acid sequence having at least 80% sequence identity to residues 198-344 of SEQ ID NO: 5.

33. The MTEM of any one of claims 20-32, wherein said second subunit comprises a residue corresponding to residue 210 of SEQ ID NO: 5.

34. The MTEM of any one of claims 20-33, wherein said second subunit comprises a residue corresponding to residue 271 of SEQ ID NO: 5.

35. The MTEM of any one of claims 20-34, wherein said second subunit comprises residues 198-344 of SEQ ID NO: 5.

36. The MTEM of any one of claims 20-35, wherein said engineered meganuclease is a single-chain meganuclease comprising a linker, wherein said linker covalently joins said first subunit and said second subunit.

37. The MTEM of any one of claims 20-36, wherein said engineered meganuclease comprises an amino acid sequence having at least 80% sequence identity to SEQ ID NO: 5.

38. The MTEM of any one of claims 20-37, wherein said engineered meganuclease comprises an amino acid sequence of SEQ ID NO: 5.

39. The MTEM of any one of claims 20-38, wherein said engineered meganuclease is encoded by a nucleic sequence having at least 80% sequence identity to a nucleic acid sequence of SEQ ID NO: 6.

40. The MTEM of any one of claims 20-39, wherein said engineered meganuclease is encoded by a nucleic acid sequence of SEQ ID NO: 6.

41. The MTEM of any one of claim 2-18 or 20-40, wherein said MTP comprises an amino acid sequence having at least 80% sequence identity to a sequence set forth in any one of SEQ ID NOs: 7-11.

42. The MTEM of any one of claim 2-18 or 20-41, wherein said MTP comprises an amino acid sequence set forth in any one of SEQ ID NOs: 7-11.

43. The MTEM of any one of claim 2-18 or 20-42, wherein said MTP is attached to the C-terminus of said engineered meganuclease.

44. The MTEM of any one of claim 2-18 or 20-42, wherein said MTP is attached to the N-terminus of said engineered meganuclease.

45. The MTEM of any one of claim 2-18 or 20-44, wherein said MTP is fused to said engineered meganuclease.

46. The MTEM of any one of claim 2-18 or 20-44, wherein said MTP is attached to said engineered meganuclease by a polypeptide linker.

47. The MTEM of any one of claim 2-18 or 20-42, wherein said engineered meganuclease is attached to a first MTP and a second MTP.

48. The MTEM of claim 47, wherein said first MTP and/or said second MTP comprises an amino acid sequence having at least 80% sequence identity to a sequence set forth in any one of SEQ ID NOs: 7-11.

49. The MTEM of claim 47 or claim 48, wherein said first MTP and/or said second MTP comprises an amino acid sequence set forth in any one of SEQ ID NOs: 7-11.

50. The MTEM of any one of claims 47-49, wherein said first MTP and said second MTP are identical.

51. The MTEM of any one of claims 47-49, wherein said first MTP and said second MTP are not identical.

52. The MTEM of any one of claims 47-51, wherein said first MTP and/or said second MTP is fused to said engineered meganuclease.

53. The MTEM of any one of claims 47-51, wherein said first MTP and/or said second MTP is attached to said engineered meganuclease by a polypeptide linker.

54. A polynucleotide comprising a nucleic acid sequence encoding said MTEM of any one of claim 2-18 or 20-53 or said engineered meganuclease of claim 1 or 19.

55. The polynucleotide of claim 54, wherein said polynucleotide is an mRNA.

56. The polynucleotide of claim 54 or claim 55, wherein said polynucleotide further comprises a nucleic acid sequence encoding a selectable marker.

57. The polynucleotide of claim 56, wherein said selectable marker is an antibiotic resistance gene.

58. An expression cassette comprising said polynucleotide of any one of claims 54-57.

59. The expression cassette of claim 58, wherein said polynucleotide comprises a promoter that is operably linked to said nucleic acid sequence encoding said MTEM or said engineered meganuclease.

60. The expression cassette of claim 59, wherein said promoter is active in a plant cell.

61. The expression cassette of claim 59 or claim 60, wherein said promoter is an anther-specific promoter, an anther-preferred promoter, a pollen-specific promoter, or a pollen-preferred promoter.

62. The expression cassette of claim 59 or claim 60, wherein said promoter is a constitutively active promoter.

63. An expression cassette comprising a polynucleotide comprising a nucleic acid sequence encoding a mitochondria-targeting engineered nuclease (MTEN), wherein said MTEN comprises an engineered nuclease attached to an MTP, and wherein said MTEN binds and cleaves and recognition sequence in a male-essential plant mitochondrial gene.

64. The expression cassette of claim 61, wherein said MTP comprises an amino acid sequence having at least 80% sequence identity to a sequence set forth in any one of SEQ ID NOs: 7-11.

65. The expression cassette of claim 63 or claim 64, wherein said MTP comprises an amino acid sequence set forth in any one of SEQ ID NOs: 7-11.

66. The expression cassette of any one of claims 63-65, wherein said male-essential plant mitochondrial gene is an mtATP gene.

67. The expression cassette of any one of claims 63-66, wherein said male-essential plant mitochondrial gene is an mtATP1 gene.

68. The expression cassette of any one of claims 63-67, wherein said engineered nuclease is an engineered meganuclease, a CRISPR system nuclease, a compact TALEN, or a megaTAL.

69. The expression cassette of any one of claims 63-68, wherein said MTP is attached to the C-terminus of said engineered nuclease.

70. The expression cassette of any one of claims 63-68, wherein said MTP is attached to the N-terminus of said engineered nuclease.

71. The expression cassette of any one of claims 63-70, wherein said MTP is fused to said engineered nuclease.

72. The expression cassette of any one of claims 63-70, wherein said MTP is attached to said engineered nuclease by a polypeptide linker.

73. The expression cassette of any one of claims 63-68, wherein said engineered nuclease is attached to a first MTP and a second MTP, wherein at least one of said first MTP and said second MTP is said MTP of claim 64 or claim 65.

74. The expression cassette of claim 73, wherein said first MTP and said second MTP are identical.

75. The expression cassette of claim 73, wherein said first MTP and said second MTP are not identical.

76. The expression cassette of any one of claims 73-75, wherein said first MTP and/or said second MTP is fused to said engineered nuclease.

77. The expression cassette of any one of claims 73-75, wherein said first MTP and/or said second MTP is attached to said engineered nuclease by a polypeptide linker.

78. The expression cassette of any one of claims 63-67, wherein said engineered nuclease is a zinc finger nuclease or a TALEN.

79. The expression cassette of claim 78, wherein said MTP is attached to the N-terminus of said engineered nuclease.

80. The expression cassette of claim 78 or claim 79, wherein said MTP is fused to said engineered nuclease.

81. The expression cassette of claim 78 or claim 79, wherein said MTP is attached to said engineered nuclease by a polypeptide linker.

82. The expression cassette of any one of claims 63-77, wherein said recognition sequence comprises SEQ ID NO: 1.

83. The expression cassette of claim 82, wherein said MTEN is said MTEM of any one of claims 2-18.

84. The expression cassette of any one of claims 63-77, wherein said recognition sequence comprises SEQ ID NO: 2.

85. The expression cassette of claim 84, wherein said MTEN is said MTEM of any one of claims 20-40.

86. The expression cassette of any one of claims 82-85, wherein said expression cassette comprises a promoter that is operably linked to said nucleic acid sequence encoding said MTEN.

87. The expression cassette of claim 86, wherein said promoter is active in a plant cell.

88. The expression cassette of claim 86 or claim 87, wherein said promoter is an anther-specific promoter, an anther-preferred promoter, a pollen-specific promoter, or a pollen-preferred promoter.

89. The expression cassette of any one of claims 86-88, wherein said promoter is a constitutively active promoter.

90. The expression cassette of any one of claims 61-79, wherein said expression cassette comprises:
(a) a first polynucleotide comprising a nucleic acid sequence encoding a first MTEN; and
(b) a second polynucleotide comprising a nucleic acid sequence encoding a second MTEN;
wherein said first MTEN and said second MTEN each comprise an engineered nuclease attached to an MTP,
wherein said first MTEN binds and cleaves a first recognition sequence in said male-essential plant mitochondrial gene, and wherein said second MTEN binds and cleaves a second recognition sequence in said male-essential plant mitochondrial gene.

91. The expression cassette of claim 90, wherein said first MTEN and said second MTEN are capable of generating cleavage sites having complementary overhangs.

92. The expression cassette of any one of claims 90-91, wherein said first recognition sequence and said second recognition sequence are less than about 1500, about 1400, about 1300, about 1200, about 1100, about 1000, about 900, about 800, about 700, about 600, about 500, about 400, about 300, about 200, about 100, or about 50 basepairs apart in said male-essential plant mitochondrial gene.

93. The expression cassette of any one of claims 61-75, 90-92, wherein said first MTEN and/or said second MTEN is an MTEM.

94. The expression cassette of claim 93, wherein said first recognition sequence and said second recognition sequence comprise identical 4 basepair center sequences.

95. The expression cassette of claim 93 or claim 94, wherein said first recognition sequence comprises SEQ ID NO: 1.

96. The expression cassette of any one of claims 93-95, wherein said second recognition sequence comprises SEQ ID NO: 2.

97. The expression cassette of any one of claims 93-96, wherein said first MTEN is said MTEM of any one of claims 2-18.

98. The expression cassette of any one of claims 93-97, wherein said second MTEN 15 said MTEM of any one of claims 20-40.

99. The expression cassette of any one of claims 90-98, wherein said expression cassette comprises a promoter that is operably linked to said nucleic acid sequence encoding said first MTEN and said nucleic acid sequence encoding said second MTEN.

100. The expression cassette of claim 99, wherein said nucleic acid sequence encoding said first MTEN and said second nucleic acid sequence encoding said second MTEN are separated by an IRES or 2A sequence.

101. The expression cassette of claim 89, wherein said 2A sequence is a T2A, a P2A, an E2A, or an F2A sequence.

102. The expression cassette of any one of claims 99-101, wherein said promoter is active in a plant cell.

103. The expression cassette of any one of claims 99-102, wherein said promoter is an anther-specific promoter, an anther-preferred promoter, a pollen-specific promoter, or a pollen-preferred promoter.

104. The expression cassette of any one of claims 99-103, wherein said promoter is a constitutively active promoter.

105. The expression cassette of any one of claims 90-98, wherein said expression cassette comprises a first promoter that is operably linked to said nucleic acid sequence encoding said first MTEN, and a second promoter that is operably linked to said nucleic acid sequence encoding said second MTEN.

106. The expression cassette of claim 105, wherein said first promoter and said second promoter are identical.

107. The expression cassette of claim 105, wherein said first promoter and said second promoter are not identical.

108. The expression cassette of any one of claims 105-107, wherein said first promoter and/or said second promoter is active in a plant cell.

109. The expression cassette of any one of claims 105-108, wherein said first promoter and/or said second promoter is an anther-specific promoter, an anther-preferred promoter, a pollen-specific promoter, or a pollen-preferred promoter.

110. The expression cassette of any one of claims 105-109, wherein said first promoter and/or said second promoter is a constitutively active promoter.

111. A recombinant DNA construct comprising a polynucleotide comprising said expression cassette of any one of claims 58-62.

112. A recombinant DNA construct comprising a polynucleotide comprising said expression cassette of any one of claims 63-110.

113. A bacterium comprising said recombinant DNA construct of claim 111.

114. The bacterium of claim 113, wherein said bacterium is Agrobacterium tumefaciens.

115. A bacterium comprising said recombinant DNA construct of claim 112.

116. The bacterium of claim 115, wherein said bacterium is Agrobacterium tumefaciens.

117. A recombinant virus comprising a polynucleotide comprising said expression cassette of any one of claims 58-62.

118. The recombinant virus of claim 117, wherein said recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

119. A recombinant virus comprising a polynucleotide comprising said expression cassette of any one of claims 63-110.

120. The recombinant virus of claim 119, wherein said recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

121. A polynucleotide comprising a sequence set forth in SEQ ID NO: 14.

122. The polynucleotide of claim 121, wherein said polynucleotide is a plant mtATP1 gene comprising a sequence set forth in SEQ ID NO: 15.

123. A genetically-modified plant cell comprising said polynucleotide of any one of claims 54-57.

124. A genetically-modified plant cell comprising said expression cassette of any one of claims 63-110.

125. A genetically-modified plant cell comprising said recombinant DNA construct of claim 111 or claim 112.

126. A genetically-modified plant cell comprising a modified male-essential mitochondrial gene.

127. The genetically-modified plant cell of claim 126, wherein said modified male-essential mitochondrial gene is inactivated.

128. The genetically-modified plant cell of claim 126 or claim 127, wherein said modified male-essential mitochondrial gene is a modified mtATP gene.

129. The genetically-modified plant cell of any one of claims 126-128, wherein said modified male-essential mitochondrial gene is a modified mtATP1 gene.

130. The genetically-modified plant cell of claim 129, wherein said modified mtATP1 gene comprises a nucleic acid sequence set forth in SEQ ID NO: 14.

131. The genetically-modified plant cell of any one of claims 123-130, wherein said genetically-modified plant cell is a genetically-modified tobacco cell, tomato cell, blackberry cell, raspberry cell, cucumber cell, watermelon cell, pomegranate cell, and grape cell.

132. The genetically-modified plant cell of any one of claims 123-131, wherein said genetically-modified plant cell comprises a maintainer construct on a nuclear chromosome, wherein said maintainer construct comprises:
(a) a copy of said male-essential mitochondrial gene which encodes a wild-type polypeptide;
(b) a non-male promoter operably linked to said copy of said male-essential mitochondrial gene; and
(c) a nucleic acid sequence encoding a maintainer MTP which is attached to said wild-type polypeptide.

133. The genetically-modified plant cell of claim 132, wherein said copy of said male-essential mitochondrial gene in said maintainer construct is codon-optimized for expression in the nucleus and encodes a wild-type polypeptide.

134. The genetically-modified plant cell of claim 132 or claim 133, wherein said copy of said male-essential mitochondrial gene in said maintainer construct encodes a wild-type polypeptide but is modified to not comprise said recognition sequence, said first recognition sequence, or said second recognition sequence.

135. The genetically-modified plant cell of any one of claims 132-134, wherein said maintainer MTP is attached to the N-terminus of said wild-type polypeptide.

136. The genetically-modified plant cell of any one of claims 132-134, wherein said maintainer MTP is attached to the C-terminus of said wild-type polypeptide.

137. The genetically-modified plant cell of any one of claims 132-36, wherein said maintainer MTP is fused to said wild-type polypeptide.

138. The genetically-modified plant cell of any one of claims 132-136, wherein said maintainer MTP is attached to said wild-type polypeptide by a polypeptide linker.

139. The genetically-modified plant cell of any one of claims 132-138, wherein said maintainer MTP comprises an amino acid sequence having at least 80% sequence identity to a sequence set forth in any one of SEQ ID NOs: 7-11.

140. The genetically-modified plant cell of any one of claims 132-139, wherein said maintainer MTP comprises an amino acid sequence set forth in any one of SEQ ID NOs: 7-11.

141. The genetically-modified plant cell of any one of claims 132-140, wherein said maintainer MTP comprises an amino acid sequence set forth in SEQ ID NO: 7.

142. The genetically-modified plant cell of any one of claims 132-141, wherein said non-male promoter is a weak non-male promoter.

143. The genetically-modified plant cell of any one of claims 132-142, wherein said non-male promoter is a CaMV35S promoter or an enhanced CaMV35S promoter.

144. The genetically-modified plant cell of any one of claims 132-141, wherein said non-male promoter is a strong non-male promoter.

145. The genetically-modified plant cell of claim 144, wherein said strong non-male promoter expresses said male-essential mitochondrial gene in comparable levels to the levels of the male-essential mitochondrial gene expressed from the mitochondrial gene.

146. The genetically-modified plant cell of any one of claims 132-142, wherein said genetically-modified plant cell comprises a restorer construct on a nuclear chromosome, wherein said restorer construct comprises:
(a) a copy of said male-essential mitochondrial gene which encodes a wild-type polypeptide;
(b) a ubiquitous promoter operably linked to said copy of said male-essential mitochondrial gene; and
(c) a nucleic acid sequence encoding a restorer MTP which is attached to said wild-type polypeptide.

147. The genetically-modified plant cell of claim 146, wherein said copy of said male-essential mitochondrial gene in said restorer construct is codon-optimized for expression in the nucleus and encodes a wild-type polypeptide.

148. The genetically-modified plant cell of claim 146 or claim 147, wherein said copy of said male-essential mitochondrial gene in said restorer construct encodes a wild-type polypeptide but is modified to not comprise said recognition sequence, said first recognition sequence, or said second recognition sequence.

149. The genetically-modified plant cell of any one of claims 146-148, wherein said restorer MTP is attached to the N-terminus of said wild-type polypeptide.

150. The genetically-modified plant cell of any one of claims 146-148, wherein said restorer MTP is attached to the C-terminus of said wild-type polypeptide.

151. The genetically-modified plant cell of any one of claims 146-150, wherein said restorer MTP is fused to said wild-type polypeptide.

152. The genetically-modified plant cell of any one of claims 146-150, wherein said restorer MTP is attached to said wild-type polypeptide by a polypeptide linker.

153. The genetically-modified plant cell of any one of claims 146-152, wherein said restorer MTP comprises an amino acid sequence having at least 80% sequence identity to a sequence set forth in any one of SEQ ID NOs: 7-11.

154. The genetically-modified plant cell of any one of claims 146-153 wherein said restorer MTP comprises an amino acid sequence set forth in any one of SEQ ID NOs: 7-11.

155. The genetically-modified plant cell of any one of claims 146-154, wherein said restorer MTP comprises an amino acid sequence set forth in SEQ ID NO: 7.

156. The genetically-modified plant cell of any one of claims 146-155, wherein said ubiquitous promoter is a weak ubiquitous promoter.

157. The genetically-modified plant cell of any one of claims 146-155, wherein said ubiquitous promoter is an mtATP promoter.

158. The genetically-modified plant cell of any one of claims 146-157, wherein said ubiquitous promoter is a β-ATP promoter.

159. The genetically-modified plant cell of any one of claims 146-155, wherein said ubiquitous promoter is a strong ubiquitous promoter.

160. The genetically-modified plant cell of claim 159, wherein said strong ubiquitous promoter is a ubiquitin promoter.

161. A plant or plant part comprising said genetically-modified plant cell of any one of claims 123-160.

162. The plant or plant part of claim 161, wherein said plant part is a seed comprising said genetically-modified plant cell.

163. A maintainer plant cell that comprises a maintainer construct on a nuclear chromosome, wherein said maintainer construct comprises:
(a) a copy of a male-essential mitochondrial gene which encodes a wild-type polypeptide;
(b) a non-male promoter operably linked to said copy of said male-essential mitochondrial gene; and
(c) a nucleic acid sequence encoding a maintainer MTP which is attached to said wild-type polypeptide.

164. The maintainer plant cell of claim 163, wherein said copy of said male-essential mitochondrial gene in said maintainer construct is codon-optimized for expression in the nucleus and encodes a wild-type polypeptide.

165. The maintainer plant cell of claim 163 or claim 164, wherein said copy of said male-essential mitochondrial gene in said maintainer construct encodes a wild-type polypeptide but is modified to not comprise said recognition sequence, said first recognition sequence, or said second recognition sequence.

166. The maintainer plant cell of any one of claims 163-165, wherein said maintainer MTP is attached to the N-terminus of said wild-type polypeptide.

167. The maintainer plant cell of any one of claims 163-165, wherein said maintainer MTP is attached to the C-terminus of said wild-type polypeptide.

168. The maintainer plant cell of any one of claims 163-167, wherein said maintainer MTP is fused to said wild-type polypeptide.

169. The maintainer plant cell of any one of claims 163-167, wherein said maintainer MTP is attached to said wild-type polypeptide by a polypeptide linker.

170. The maintainer plant cell of any one of claims 163-169, wherein said maintainer MTP comprises an amino acid sequence having at least 80% sequence identity to a sequence set forth in any one of SEQ ID NOs: 7-11.

171. The maintainer plant cell of any one of claims 163-170, wherein said maintainer MTP comprises an amino acid sequence set forth in any one of SEQ ID NOs: 7-11.

172. The maintainer plant cell of any one of claims 163-171, wherein said maintainer MTP comprises an amino acid sequence set forth in SEQ ID NO: 7.

173. The maintainer plant cell of any one of claims 163-172, wherein said non-male promoter is a strong non-male promoter.

174. The maintainer plant cell of claim 173, wherein said strong non-male promoter expresses said male-essential mitochondrial gene in comparable levels to the levels of the male-essential mitochondrial gene expressed from the mitochondrial gene.

175. The maintainer plant cell of any one of claims 163-172, wherein said non-male promoter is a weak non-male promoter.

176. The maintainer plant cell of any one of claims 163-172, wherein said non-male promoter is a CaMV35S promoter or an enhanced CaMV35S promoter.

177. A maintainer plant or maintainer plant part comprising said maintainer plant cell of any one of claims 163-174.

178. The maintainer plant or maintainer plant part of claim 177, wherein said maintainer plant part is a seed comprising said maintainer plant cell.

179. A maintainer plant or maintainer plant part comprising said maintainer plant cell of any one of claim 163-172, 175, or 176.

180. The maintainer plant or maintainer plant part of claim 179, wherein said maintainer plant part is a seed comprising said maintainer plant cell.

181. A restorer plant cell that comprises a restorer construct on a nuclear chromosome, wherein said restorer construct comprises:
(a) a copy of a male-essential mitochondrial gene which encodes a wild-type polypeptide;
(b) a ubiquitous promoter operably linked to said copy of said male-essential mitochondrial gene; and
(c) a nucleic acid sequence encoding a restorer MTP which is attached to said wild-type polypeptide.

182. The restorer plant cell of claim 181, wherein said copy of said male-essential mitochondrial gene in said restorer construct is codon-optimized for expression in the nucleus and encodes a wild-type polypeptide.

183. The restorer plant cell of claim 181 or claim 182, wherein said copy of said male-essential mitochondrial gene in said restorer construct encodes a wild-type polypeptide but is modified to not comprise said recognition sequence, said first recognition sequence, or said second recognition sequence.

184. The restorer plant cell of any one of claims 181-183, wherein said restorer MTP is attached to the N-terminus of said wild-type polypeptide.

185. The restorer plant cell of any one of claims 181-183, wherein said restorer MTP is attached to the C-terminus of said wild-type polypeptide.

186. The restorer plant cell of any one of claims 181-185, wherein said restorer MTP is fused to said wild-type polypeptide.

187. The restorer plant cell of any one of claims 181-185, wherein said restorer MTP is attached to said wild-type polypeptide by a polypeptide linker.

188. The restorer plant cell of any one of claims 181-187, wherein said restorer MTP comprises an amino acid sequence having at least 80% sequence identity to a sequence set forth in any one of SEQ ID NOs: 7-11.

189. The restorer plant cell of any one of claims 181-188, wherein said restorer MTP comprises an amino acid sequence set forth in any one of SEQ ID NOs: 7-11.

190. The restorer plant cell of any one of claims 181-189, wherein said restorer MTP comprises an amino acid sequence set forth in SEQ ID NO: 7.

191. The restorer plant cell of any one of claims 181-190, wherein said ubiquitous promoter is a strong ubiquitous promoter.

192. The restorer plant cell of claim 191, wherein said strong ubiquitous promoter is a ubiquitin promoter.

193. The restorer plant cell of any one of claims 181-190, wherein said ubiquitous promoter is a weak ubiquitous promoter.

194. The restorer plant cell of any one of claims 181-190, wherein said ubiquitous promoter is an mtATP promoter.

195. The restorer plant cell of any one of claims 181-190, wherein said ubiquitous active promoter is a β-ATP promoter.

196. A restorer plant or restorer plant part comprising said restorer plant cell of any one of claims 181-192.

197. The restorer plant or restorer plant part of claim 196, wherein said restorer plant part is a seed comprising said restorer plant cell.

198. A restorer plant or restorer plant part comprising said restorer plant cell of any one of claim 181-191 or 193-195.

199. The restorer plant or restorer plant part of claim 198, wherein said restorer plant part is a seed comprising said restorer cell.

200. A method for producing a genetically-modified plant cell, said method comprising introducing into a plant cell:
(a) a polynucleotide comprising a nucleic acid sequence encoding said MTEM of any one of claims 1-51, wherein said MTEM is expressed in said plant cell; or
(b) said MTEM of any one of claim 2-18 or 20-53;
wherein said MTEM produces a cleavage site at said recognition sequence in said mtATP1 gene in mitochondrial genomes.

201. The method of claim 200, wherein said cleavage site is repaired, such that said recognition sequence comprises an insertion or deletion.

202. The method of claim 201, wherein said insertion or deletion inactivates said mtATP1 gene.

203. The method of any one of claims 200-202, wherein said polynucleotide is an mRNA.

204. The method of claim 203, wherein said polynucleotide is said mRNA of claim 55.

205. The method of any one of claims 200-202, wherein said polynucleotide is a recombinant DNA construct.

206. The method of claim 205, wherein said polynucleotide is said recombinant DNA construct of claim 111.

207. The method of any one of claims 200-202, wherein said polynucleotide is introduced into said plant cell by a recombinant virus.

208. The method of claim 207, wherein said recombinant virus is said recombinant virus of claim 117 or claim 118.

209. The method of any one of claims 200-202, wherein said polynucleotide is introduced into said plant cell by said bacterium of claim 113 or claim 114.

210. The method of any one of claims 200-202, wherein said polynucleotide is introduced into said plant cell by Agrobacterium-mediated transformation, biolistic transformation, by microinjection, or by electroporation.

211. The method of any one of claims 200-210, wherein said polynucleotide comprises said expression cassette of any one of claims 58-62.

212. A method for producing a genetically-modified plant cell, said method comprising introducing into a plant cell:
(a) a polynucleotide comprising a nucleic acid sequence encoding an MTEN, wherein said MTEN is expressed in said plant cell; or
(b) said MTEN;
wherein said MTEN comprises an engineered nuclease attached to an MTP,
wherein said MTEN binds and cleaves a recognition sequence in a male-essential plant mitochondrial gene to generate a cleavage site.

213. The method of claim 212, wherein said cleavage site is repaired, such that said recognition sequence comprises an insertion or deletion.

214. The method of claim 213, wherein said insertion or deletion inactivates said male-essential plant mitochondrial gene.

215. The method of any one of claims 212-214, wherein said MTP comprises an amino acid sequence having at least 80% sequence identity to a sequence set forth in any one of SEQ ID NOs: 7-11.

216. The method of any one of claims 212-215, wherein said MTP comprises an amino acid sequence set forth in any one of SEQ ID NOs: 7-11.

217. The method of any one of claims 212-216, wherein said male-essential plant mitochondrial gene is an mtATP gene.

218. The method of any one of claims 212-217, wherein said male-essential plant mitochondrial gene is an mtATP1 gene.

219. The method of any one of claims 212-218, wherein said engineered nuclease is an engineered meganuclease, a CRISPR system nuclease, a compact TALEN, or a megaTAL.

220. The method of any one of claims 212-219, wherein said MTP is attached to the C-terminus of said engineered nuclease.

221. The method of any one of claims 212-219, wherein said MTP is attached to the N-terminus of said engineered nuclease.

222. The method of any one of claims 212-221, wherein said MTP is fused to said engineered nuclease.

223. The method of any one of claims 212-221, wherein said MTP is attached to said engineered nuclease by a polypeptide linker.

224. The method of any one of claims 212-219, wherein said engineered nuclease is attached to a first MTP and a second MTP, wherein at least one of said first MTP and said second MTP is said MTP of claim 215 or claim 216.

225. The method of claim 224, wherein said first MTP and said second MTP are identical.

226. The method of claim 224, wherein said first MTP and said second MTP are not identical.

227. The method of any one of claims 224-226, wherein said first MTP and/or said second MTP is fused to said engineered nuclease.

228. The method of any one of claims 224-226, wherein said first MTP and/or said second MTP is attached to said engineered nuclease by a polypeptide linker.

229. The method of any one of claims 212-218, wherein said engineered nuclease is a zinc finger nuclease or a TALEN.

230. The method of claim 229, wherein said MTP is attached to the N-terminus of said engineered nuclease.

231. The method of claim 229 or claim 230, wherein said MTP is fused to said engineered nuclease.

232. The method of claim 229 or claim 230 wherein said MTP is attached to said engineered nuclease by a polypeptide linker.

233. The method of any one of claims 212-232, wherein said recognition sequence comprises SEQ ID NO: 1.

234. The method of claim 233, wherein said MTEN is said MTEM of any one of claims 2-18.

235. The method of any one of claims 212-232, wherein said recognition sequence comprises SEQ ID NO: 2.

236. The method of claim 235, wherein said MTEN is said MTEM of any one of claims 20-40.

237. The method of any one of claims 212-236, wherein said polynucleotide comprises a promoter that is operably linked to said nucleic acid sequence encoding said MTEN.

238. The method of claim 237, wherein said promoter is active in a plant cell.

239. The method of claim 237 or claim 238, wherein said promoter is an anther-specific promoter, an anther-preferred promoter, a pollen-specific promoter, or a pollen-preferred promoter.

240. The method of any one of claims 237-239, wherein said promoter is a constitutively active promoter.

241. The method of any one of claims 212-240, wherein said polynucleotide is introduced into said plant cell by a recombinant virus.

242. The method of claim 241, wherein said recombinant virus is said recombinant virus of any one of claims 117-120.

243. The method of any one of claims 212-240, wherein said polynucleotide is introduced into said plant cell by said bacterium of any one of claims 113-116.

244. The method of any one of claims 212-240, wherein:
(a) said polynucleotide is introduced into said plant cell by Agrobacterium-mediated transformation, biolistic transformation, by microinjection, or by electroporation; or
(b) said MTEN is introduced by biolistic transformation, by microinjection, or by electroporation.

245. The method of any one of claims 212-244, wherein an expression cassette is introduced into said plant cell that comprises said polynucleotide.

246. The method of claim 245, wherein said expression cassette is said expression cassette of any one of claims 63-110.

247. The method of claim 245 or claim 246, wherein said expression cassette is introduced into said plant cell using a recombinant DNA construct.

248. The method of claim 245 or claim 246, wherein said expression cassette is introduced into said plant cell using a recombinant virus.

249. The method of claim 248, wherein said recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

250. The method of claim 245 or claim 246, wherein said expression cassette is introduced into said plant cell using a bacterium.

251. The method of claim 250, wherein said bacterium is Agrobacterium tumefaciens.

252. The method of claim 245 or claim 246, wherein said expression cassette is introduced into said plant cell by Agrobacterium-mediated transformation, biolistic transformation, by microinjection, or by electroporation.

253. The method of any one of claims 212-252, said method comprising introducing into said plant cell:
(a) a first MTEN, or a first polynucleotide comprising a nucleic acid sequence encoding said first MTEN, wherein said first MTEN is express in said plant cell; and
(b) a second MTEN, or a second polynucleotide comprising a nucleic acid sequence encoding said second MTEN, wherein said second MTEN is expressed in said plant cell;
wherein said first MTEN and said second MTEN each comprise an engineered nuclease attached to an MTP,
wherein said first MTEN binds and cleaves a first recognition sequence in said male-essential plant mitochondrial gene to generate a first cleavage site, and wherein said second MTEN binds and cleaves a second recognition sequence in said male-essential plant mitochondrial gene to generate a second cleavage site.

254. The method of claim 253, wherein said first cleavage site and said second cleavage site are repaired, such that said first recognition sequence and said second recognition sequence comprise an insertion or deletion.

255. The method of claim 254, wherein said insertion or deletion inactivates said male-essential plant mitochondrial gene.

256. The method of claim 253, wherein the intervening genomic sequence between said first cleavage site and said second cleavage site is removed.

257. The method of claim 256, wherein said first cleavage site and said second cleavage site ligate to one another to anneal the mitochondrial genome to generate a modified male-essential plant mitochondrial gene.

258. The method of any one of claims 253-257, wherein said first MTEN and said second MTEN are capable of generating cleavage sites having complementary overhangs.

259. The method of any one of claims 253-258, wherein said first recognition sequence and said second recognition sequence are less than about 1500, about 1400, about 1300, about 1200, about 1100, about 1000, about 900, about 800, about 700, about 600, about 500, about 400, about 300, about 200, about 100, or about 50 basepairs apart in said male-essential plant mitochondrial gene.

260. The method of any one of claims 253-259 wherein said first MTEN and/or said second MTEN is an MTEM.

261. The method of claim 260, wherein said first recognition sequence and said second recognition sequence comprise identical 4 basepair center sequences.

262. The method of claim 260 or claim 261, wherein said first recognition sequence comprises SEQ ID NO: 1.

263. The method of any one of claims 260-262, wherein said second recognition sequence comprises SEQ ID NO: 2.

264. The method of any one of claims 260-263, wherein said first MTEN is said MTEM of any one of claims 2-18.

265. The method of any one of claims 260-264, wherein said second MTEN is said MTEM of any one of claims 20-40.

266. The method of any one of claims 253-265, wherein said first MTEN is operably linked to a first promoter, and wherein said second MTEN is operably linked to a second promoter.

267. The method of claim 266, wherein said first promoter and said second promoter are identical.

268. The method of claim 266, wherein said first promoter and said second promoter are not identical.

269. The method of any one of claims 266-268, wherein said first promoter and/or said second promoter is active in a plant cell.

270. The method of any one of claims 266-269, wherein said first promoter and/or said second promoter is an anther-specific promoter, an anther-preferred promoter, a pollen-specific promoter, or a pollen-preferred promoter.

271. The method of any one of claims 266-270, wherein said first promoter and/or said second promoter is a ubiquitous and/or constitutively active promoter.

272. The method of any one of claims 253-271, wherein said first polynucleotide and/or said second polynucleotide is an mRNA.

273. The method of claim 272, wherein said first polynucleotide and/or said second polynucleotide is said mRNA of claim 55.

274. The method of any one of claims 253-271, wherein said first polynucleotide and/or said second polynucleotide is a recombinant DNA construct.

275. The method of claim 274, wherein said first polynucleotide and/or said second polynucleotide is said recombinant DNA construct of claim 111 or claim 112.

276. The method of any one of claims 253-275, wherein said first polynucleotide and/or said second polynucleotide is introduced into said plant cell by a recombinant virus.

277. The method of claim 276, wherein said recombinant virus is said recombinant virus of any one of claims 117-120.

278. The method of any one of claims 253-275, wherein said first polynucleotide and/or said second polynucleotide is introduced into said plant cell by said bacterium of any one of claims 113-116.

279. The method of any one of claims 253-279, wherein said first polynucleotide and/or said second polynucleotide is introduced into said plant cell by Agrobacterium-mediated transformation, biolistic transformation, by microinjection, or by electroporation.

280. The method of any one of claims 253-279, wherein an expression cassette is introduced into said plant cell that comprises said first polynucleotide and said second polynucleotide.

281. The method of claim 280, wherein said expression cassette is said expression cassette of any one of claims 63-110.

282. The method of claim 280 or claim 281, wherein said expression cassette is introduced into said plant cell using a recombinant DNA construct.

283. The method of claim 280 or claim 281, wherein said expression cassette is introduced into said plant cell using a recombinant virus.

284. The method of claim 283, wherein said recombinant virus is a recombinant adenovirus, a recombinant lentivirus, a recombinant retrovirus, a recombinant adeno-associated virus (AAV), a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

285. The method of claim 280 or claim 281, wherein said expression cassette is introduced into said plant cell using a bacterium.

286. The method of claim 285, wherein said bacterium is Agrobacterium tumefaciens.

287. The method of claim 280 or claim 281, wherein said expression cassette is introduced into said plant cell by Agrobacterium-mediated transformation, biolistic transformation, by microinjection, or by electroporation.

288. The method of any one of claims 253-287, wherein said genetically-modified plant cell comprises a maintainer construct on a nuclear chromosome, wherein said maintainer construct comprises:
(a) a copy of said male-essential mitochondrial gene which encodes a wild-type polypeptide;
(b) a non-male promoter operably linked to said copy of said male-essential mitochondrial gene; and
(c) a nucleic acid sequence encoding a maintainer MTP which is attached to said wild-type polypeptide.

289. The method of claim 288, wherein said copy of said male-essential mitochondrial gene in said maintainer construct is codon-optimized for expression in the nucleus and encodes a wild-type polypeptide.

290. The method of claim 288 or claim 289, wherein said copy of said male-essential mitochondrial gene in said maintainer construct encodes a wild-type polypeptide but is modified to not comprise said recognition sequence, said first recognition sequence, or said second recognition sequence.

291. The method of any one of claims 288-290, wherein said maintainer MTP is attached to the N-terminus of said wild-type polypeptide.

292. The method of any one of claims 288-290, wherein said maintainer MTP is attached to the C-terminus of said wild-type polypeptide.

293. The method of any one of claims 288-292, wherein said maintainer MTP is fused to said wild-type polypeptide.

294. The method of any one of claims 288-292, wherein said maintainer MTP is attached to said wild-type polypeptide by a polypeptide linker.

295. The method of any one of claims 288-294, wherein said maintainer MTP comprises an amino acid sequence having at least 80% sequence identity to a sequence set forth in any one of SEQ ID NOs: 7-11.

296. The method of any one of claims 288-295, wherein said maintainer MTP comprises an amino acid sequence set forth in any one of SEQ ID NOs: 7-11.

297. The method of any one of claims 288-296, wherein said maintainer MTP comprises an amino acid sequence set forth in SEQ ID NO: 7.

298. The method of any one of claims 288-297, wherein said non-male promoter is a weak non-male promoter.

299. The method of any one of claims 288-298, wherein said non-male promoter is a CaMV35S promoter or an enhanced CaMV35S promoter.

300. The method of any one of claims 288-297, wherein said non-male promoter is a strong non-male promoter.

301. The method of claim 300, wherein said strong non-male promoter is a ubiquitin promoter.

302. The method of any one of claims 200-301, wherein said genetically-modified plant cell is cultured into a genetically-modified plant comprising said modified male-essential plant mitochondrial gene.

303. The method of claim 302, wherein said genetically-modified plant cell is a genetically-modified tobacco cell.

304. The method of any one of claims 288-301, wherein said genetically-modified plant cell is cultured into a genetically-modified plant comprising said modified male-essential plant mitochondrial gene and said maintainer construct on a nuclear chromosome.

305. The method of claim 304, wherein said genetically-modified plant cell is a genetically-modified tobacco cell.

306. The method of claim 298 or claim 299, wherein said genetically-modified plant cell is cultured into a genetically-modified plant comprising said modified male-essential plant mitochondrial gene and said maintainer construct on a nuclear chromosome, and wherein said genetically-modified plant is unable to produce mature seed.

307. The method of claim 306, wherein said genetically-modified plant produces seedless fruit.

308. A genetically-modified plant cell made by the method of any one of claims 200-301.

309. A genetically-modified plant made by the method of claim 302 or claim 303.

310. A genetically-modified plant made by the method of claim 304 or claim 305.

311. A genetically-modified plant made by the method of claim 306 or claim 307.

312. The genetically-modified plant of claim 311, wherein said genetically-modified plant is unable to produce mature seed.

313. The genetically-modified plant of claim 312, wherein said genetically-modified plant produces seedless fruit.

314. A method of producing hybrid seed, said method comprising:
(a) crossing said genetically-modified plant of claim 310 with a restorer plant comprising a restorer construct on a nuclear chromosome; and
(b) culturing said crossed plant to produce hybrid seed;
wherein said restorer construct comprises:
(i) a copy of said male-essential mitochondrial gene which encodes a wild-type polypeptide;
(ii) a ubiquitous promoter operably linked to said copy of said male-essential mitochondrial gene; and
(iii) a nucleic acid sequence encoding a restorer MTP which is attached to said wild-type polypeptide;
wherein said hybrid seed comprises said maintainer construct on a nuclear chromosome, said restorer construct on a nuclear chromosome, and said modified male-essential plant mitochondrial gene.

315. The method of claim 314, wherein said copy of said male-essential mitochondrial gene in said restorer construct is codon-optimized for expression in the nucleus and encodes a wild-type polypeptide.

316. The method of claim 314 or claim 315, wherein said copy of said male-essential mitochondrial gene in said restorer construct encodes a wild-type polypeptide but is modified to not comprise said recognition sequence, said first recognition sequence, or said second recognition sequence.

317. The method of any one of claims 314-316, wherein said restorer MTP is attached to the N-terminus of said wild-type polypeptide.

318. The method of any one of claims 314-316, wherein said restorer MTP is attached to the C-terminus of said wild-type polypeptide.

319. The method of any one of claims 314-318, wherein said restorer MTP is fused to said wild-type polypeptide.

320. The method of any one of claims 314-318, wherein said restorer MTP is attached to said wild-type polypeptide by a polypeptide linker.

321. The method of any one of claims 314-320, wherein said restorer MTP comprises an amino acid sequence having at least 80% sequence identity to a sequence set forth in any one of SEQ ID NOs: 7-11.

322. The method of any one of claims 314-321, wherein said restorer MTP comprises an amino acid sequence set forth in any one of SEQ ID NOs: 7-11.

323. The method of any one of claims 314-321, wherein said restorer MTP comprises an amino acid sequence set forth in SEQ ID NO: 7.

324. The method of any one of claims 314-323, wherein said ubiquitous promoter is a weak ubiquitous promoter.

325. The method of any one of claims 314-324, wherein said ubiquitous promoter is an mtATP promoter.

326. The method of any one of claims 314-325, wherein said ubiquitous promoter is a β-ATP promoter.

327. The method of any one of claims 314-323, wherein said ubiquitous promoter is a strong ubiquitous promoter.

328. The method of any one of claims 314-323, wherein said ubiquitous promoter is a ubiquitin promoter.

329. The method of any one of claims 314-328, wherein said genetically-modified plant of claim 310 and said restorer plant are inbred plants.

330. The method of claim 329 wherein the said genetically-modified plant of claim 310 and said restorer plant are genetically diverse.

331. The method of any one of claims 314-330, wherein said genetically-modified plant and said restorer plant are tobacco plants, tomato plants, blackberry plants, raspberry plants, cucumber plants, watermelon plants, pomegranate plants, and grape plants.

332. Hybrid seed produced by the method of any one of claims 314-329.

333. A method of producing seed of a plant comprising a cytoplasmic male sterility trait, said method comprising:
(a) crossing said genetically-modified plant of claim 310 with a maintainer plant comprising said maintainer construct on a nuclear chromosome and said male-essential plant mitochondrial gene; and
(b) culturing said crossed plant to produce seed;
wherein said seed comprises said maintainer construct on a nuclear chromosome and said modified male-essential plant mitochondrial gene.

334. The method of claim 331, wherein said genetically-modified plant and said maintainer plant are tobacco plants, tomato plants, blackberry plants, raspberry plants, cucumber plants, watermelon plants, pomegranate plants, and grape plants.

335. A method of producing seedless fruit, said method comprising:
(a) pollinating said genetically-modified plant of any one of claims 311-313 with pollen from a wild-type plant or said maintainer plant of any of claims 179-180; and
(b) culturing the pollinated plant in order to obtain said seedless fruit.

336. An organelle-targeting engineered nuclease (OTEN) capable of binding and cleaving a recognition sequence in an organelle genome of a eukaryotic cell, wherein said OTEN comprises an engineered nuclease attached to a mitochondrial transit peptide (MTP), wherein said MTP comprises an amino acid sequence having at least 80% sequence identity to a sequence set forth in SEQ ID NO: 10.
